(19) United States
(12) Patent Application Publication
(10) Pub. No.: US 2025/0333419 A1
(43) Pub. Date: Oct. 30, 2025

(54) BICYCLIC COMPOUNDS AS INHIBITORS OF WRN

(71) Applicant: Incyte Corporation, Wilmington, DE (US)

(72) Inventors: Joe Barbosa, Wilmington, DE (US); Yanran Ai, Wilmington, DE (US); Marc Deller, Wilmington, DE (US); Balu D. Dherange, Wilmington, DE (US); Ananda Ghosh, Wilmington, DE (US); Joe Glenn, JR., Wilmington, DE (US); Leslie McQuirter, Wilmington, DE (US); Xiaozhao Wang, Wilmington, DE (US); Eddy Yue, Wilmington, DE (US); Guofeng Zhang, Wilmington, DE (US)

(73) Assignee: Incyte Corporation, Wilmington, DE (US)

(21) Appl. No.: 18/821,138

(22) Filed: Aug. 30, 2024

Related U.S. Application Data
(60) Provisional application No. 63/580,209, filed on Sep. 1, 2023.

(51) Int. Cl. C07D 487/04; A61K 31/519

(52) U.S. Cl. CPC C07D 487/04; A61K 31/519

(57) ABSTRACT

Disclosed are compounds of Formula (I), methods of using the compounds as immunomodulators, and pharmaceutical compositions comprising such compounds. The compounds are useful in treating, preventing, or ameliorating diseases or disorders such as cancer or infections.

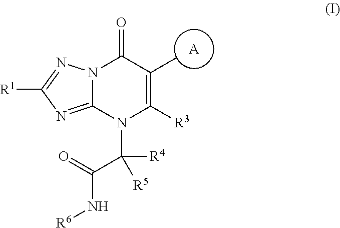

### FIELD OF THE DISCLOSURE

The present application is concerned with pharmaceutically active compounds. The disclosure provides compounds as well as their compositions and methods of use. The compounds are inhibitors of WRN and are useful in the treatment of various diseases including cancer.

### BACKGROUND

Werner helicase (WRN) belongs to the RecQ helicase family, along with BLM, RecQI, RecQ4 and RecQ5. These enzymes play broad roles in genome maintenance, preserving chromosome stability, and suppressing neoplastic transformation (Chu, W. et al. RecQ helicases: multifunctional genome caretakers. Nat Rev Cancer 9, 644-654 (2009)).

Genome-wide screens identified high dependence of WRN by microsatellite instability-high (MSI-H) cancer cells, i.e., deletion of WRN has anti-proliferation effect on MSI cells, but not microsatellite stable (MSS) cells (Chu (supra); Behan, F. M. et al. Prioritization of cancer therapeutic targets using CRISPR-Cas9 screens. Nature 568, 511-516 (2019); Chan, E. M. et al.

WRN helicase is a synthetic lethal target in microsatellite unstable cancers. Nature 568, 551-556 (2019); Kategaya, L. et al. Werner syndrome helicase is required for the survival of cancer cells with microsatellite instability. iScience 13, 488-497 (2019)).

WRN is a multifunctional enzyme having exonuclease activity, ATPase activity, and helicase activities. Site-directed mutagenesis and functional rescue studies have determined that helicase activity of WRN is critical for this dependence (Behan (supra); Chan (supra)). Therefore, molecules that targets WRN helicase activity could have therapeutic uses in MSI-H/dMMR cancers. The WRN helicase activity is driven by ATP hydrolysis, which is catalyzed by its ATPase activity. So small molecules that inhibit WRN ATPase activity, WRN ATP hydrolysis activity, or both will ultimately inhibit its WRN helicase activity.

MSI-H or mismatch repair deficient (dMMR) tumors have an accumulation of genetic errors in sequences that normally contain short repeats (called microsatellites). Nearly 4% of all cancers have MSI-H/dMMR, with the highest prevalence detected in endometrial (31%), colon (20%), and gastric cancers (19%) (Bonneville R. et al, Landscape of Microsatellite Instability Across 39 Cancer Types JCO Precision Oncology 1, 1-15 (2017)).

Recently, checkpoint inhibitors (CPI) have emerged as effective treatment for MSI-H/dMMR cancers in both 1st line and 2nd line settings, but with limited response rates (for example, Pembrolizumab approved in MSI-H/dMMR tumor agonistic >2nd line with 33.3% ORR (https://www.onclive.com/view/fda-grants-full-approval-to-pembrolizumab-for-select-patients-with-msi-h-or-dmmr-solid-tumors; Green A. K. et al. A Review of Immune Checkpoint Blockade Therapy in Endometrial Cancer. Am Soc Clin Oncol Educ Book. 40, 1-7 (2020)), and resistance has developed in some patients.

WRN inhibitors have a different mode of action compared to checkpoint inhibitors, and will have the potential to complement CPI in the treatment of MSI-H/dMMR cancers. In addition, WRN sensitivity is maintained in preclinical MSI-H colorectal cancer (CRC) models with resistance to standard of care (SOC), including targeted therapies, chemotherapy, and immunotherapy (Picco, C. et al. Werner Helicase Is a Synthetic-Lethal Vulnerability in Mismatch Repair-Deficient Colorectal Cancer Refractory to Targeted Therapies, Chemotherapy, and Immunotherapy. Cancer Discov 11, 1923-1937 (2021)). This has the potential to broaden WRN inhibitors' clinical uses.

Accordingly, there is a need for new compounds that inhibit WRN and which are useful in treating cancers such as MSI-H/dMMR cancers. This application address that need and others.

### SUMMARY

The present disclosure provides, inter alia, compounds of Formula (I):

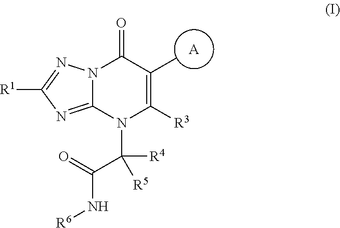

or a pharmaceutically acceptable salt thereof, wherein constituent variables are defined herein.

The present disclosure further provides a pharmaceutical composition comprising a compound disclosed herein, or a pharmaceutically acceptable salt thereof, and one or more pharmaceutically acceptable excipient or carrier.

The present disclosure further provides methods of inhibiting WRN, comprising administering to a patient a compound disclosed herein, or a pharmaceutically acceptable salt thereof. In some embodiments, the inhibition of WRN is inhibition of WRN helicase activity. In some embodiments, the inhibition of WRN is inhibition of WRN ATPase activity. In some embodiments, the inhibition of WRN is inhibition of WRN helicase activity and WRN ATPase activity.

The present disclosure further provides methods of treating a disease or disorder associated with WRN activity, comprising administering to a patient in need thereof a therapeutically effective amount of a compound of disclosed herein, or a pharmaceutically acceptable salt thereof. In some embodiments, the WRN activity is WRN helicase activity. In some embodiments, the WRN activity is WRN ATPase activity. In some embodiments, the WRN activity is WRN helicase activity and WRN ATPase activity.

The present disclosure also provides use of a compound of disclosed herein, or a pharmaceutically acceptable salt thereof, for preparation of a medicament for use in any of the methods described herein.

The present disclosure also provides a compound of disclosed herein, or a pharmaceutically acceptable salt thereof, for use in any of the methods described herein.

### BRIEF DESCRIPTION OF THE DRAWINGS

FIG. 1 graphically illustrates that the compound of Example 1 inhibits WRN helicase in a time dependent manner.

FIG. 2 graphically illustrates a mutation of Cys 727 reduces the potency of the compound of Example 1.

### DETAILED DESCRIPTION

### Compounds

The present disclosure provides, inter alia, compounds of Formula (I):

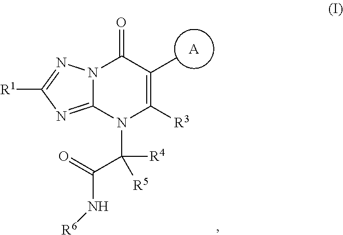

or a pharmaceutically acceptable salt thereof, wherein constituent variables are defined herein. In some embodiments, compounds of Formula (I) can be used as WRN inhibitor compounds.

In some embodiments, the compound of Formula (I) is:

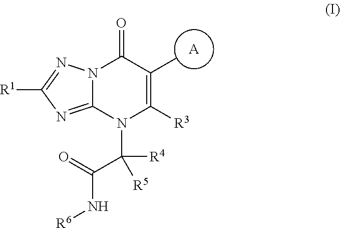

or a pharmaceutically acceptable salt thereof, wherein:
   each [symbol] is independently a single or double bond, provided that proper valency is maintained;
   Ring moiety A is selected from Formulas (A-1), (A-2), (A-3), (A-4), (A-5), and (A-6):

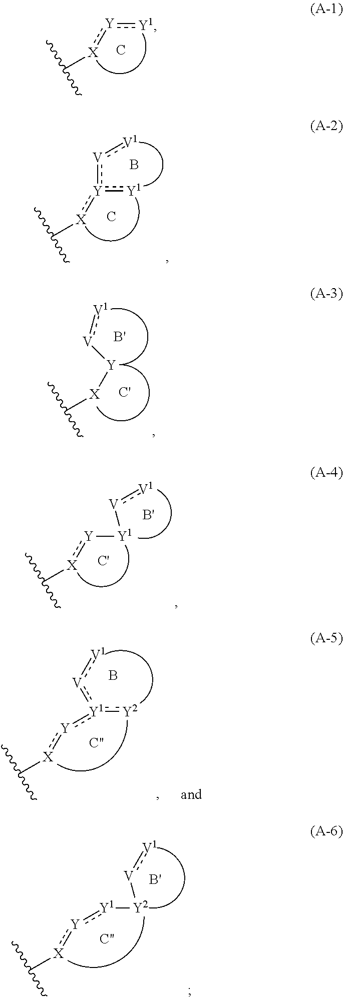

wherein Ring C is monocyclic in Formula (A-1), Ring B is fused to Y and Y¹ of Ring C in Formula (A-2), Ring B′ is spiro bound to Y in Ring C′ in Formula (A-3), Ring B′ is spiro bound to Y¹ of Ring C′ in Formula (A-4), Ring B is fused to Y¹ and Y² of Ring C″ in Formula (A-5), and Ring B′ is spiro bound to Y² in Ring C″ in Formula (A-6);
Ring B is selected from phenyl, C₃₋₇ cycloalkyl, 5-6-membered heteroaryl, or 4-7-membered heterocycloalkyl, which is optionally substituted by 1, 2, 3, or 4 independently selected R² substituents;
Ring C is selected from phenyl, C₃₋₇ cycloalkyl, 5-6-membered heteroaryl, or 4-7 membered heterocycloalkyl, which is optionally substituted by 1, 2, 3, or 4 independently selected R² substituents;
Ring B′ is 4-7-membered heterocycloalkyl or C₃₋₇ cycloalkyl, which is optionally substituted by 1, 2, 3, or 4 independently selected R² substituents;
Ring C′ is selected from 4-7 membered heterocycloalkyl or C₃₋₇ cycloalkyl, which is optionally substituted by 1, 2, 3, or 4 independently selected R² substituents;
Ring C″ is selected from 4-7 membered heterocycloalkyl or C₄₋₇ cycloalkyl, which is optionally substituted by 1, 2, 3, or 4 independently selected R² substituents; X is C, CH, or N;
when Ring A is Formula (A-1), then (i) Y is CR^{W}, CHR^{W}, or NRW; and Y¹ is CH, CH₂, N, NH, S or O; or (ii) Y is CH, CH₂, N, NH, S or O; and Y¹ is CR^{W}, CHR^{W}, or NR^{W}; provided that there are no O—O, S—S, or S—O bonds;
when Ring A is Formula (A-2), then (i) Y is C, CH, or N; Y¹ is C, CH, or N; V is CR^{W}, CHR^{W}, or NR^{W}; and V¹ is CH, CH₂, N, NH, S or O; or (ii) Y is C, CH, or N; Y¹ is C, CH, or N; V is CH, CH₂, N, NH, S or O; and V¹ is CR^{W}, CHR^{W}, or NR^{W}; provided that there are no O—O, S—S, or S—O bonds;
when Ring A is Formula (A-3), then (i) Y is C; V is CR^{W}, CHR^{W}, or NR^{W}; and V¹ is CH, CH₂, N, NH, S or O; or (ii) Y is C; V is CH, CH₂, N, NH, S or O; and V¹ is CR^{W}, CHR^{W}, or NR^{W}; provided that there are no O—O, S—S, or S—O bonds;
when Ring A is Formula (A-4), then (i) Y is CH, CH₂, N, NH, S or O; Y¹ is C; V is CR^{W}, CHR^{W}, or NR^{W}; and V¹ is CH, CH₂, N, NH, S or O; or (ii) Y is CH, CH₂, N, NH, S or O; Y¹ is C; V is CH, CH₂, N, NH, S or O; and V¹ is CR^{W}, CHR^{W}, or NR^{W}; or (iii) Y is CR^{W}, CHR^{W}, or NR^{W}; Y¹ is C; V is CH, CH₂, N, NH, S or O; and V¹ is CH, CH₂, N, NH, S or O; provided that there are no O—O, S—S, or S—O bonds;
when Ring A is Formula (A-5), then (i) Y is CR^{W}, CHR^{W}, or NR^{W}; Y¹ is C, CH, or N; Y² is C, CH, or N; V is CH, CH₂, N, NH, S or O; and V¹ is CH, CH₂, N, NH, S or O; or (ii) Y is CH, CH₂, N, NH, S or O; Y¹ is C, CH, or N; Y² is C, CH, or N; V is CR^{W}, CHR^{W}, or NR^{W}; and V¹ is CH, CH₂, N, NH, S, O, or a bond; or (iii) Y is CH, CH₂, N, NH, S or O; Y¹ is C, CH, or N; Y² is C, CH, or N; V is CH, CH₂, N, NH, S or O; and V¹ is CR^{W}, CHR^{W}, or NR^{W}; provided that there are no O—O, S—S, or S—O bonds;
when Ring A is Formula (A-6), then (i) Y is CR^{W}, CHR^{W}, or NR^{W}; Y¹ is CH, CH₂, N, NH, S or O; Y² is C; V is CH, CH₂, N, NH, S or O; and V¹ is CH, CH₂, N, NH, S or O; or (ii) Y is CH, CH₂, N, NH, S or O; Y¹ is CR^{W}, CHR^{W}, or NR^{W}; Y² is C; V is CH, CH₂, N, NH, S or O; and V¹ is CH, CH₂, N, NH, S or O; or (iii) Y is CH, CH₂, N, NH, S or O; Y¹ is CH, CH₂, N, NH, S or O; Y² is C; V is CR^{W}, CHR^{W}, or NR^{W}; and V¹ is CH, CH₂, N, NH, S or O; or (iv) Y is CH, CH₂, N, NH, S or O; Y¹ is CH, CH₂, N, NH, S or O; Y² is C; V is CH, CH₂, N, NH, S or O; and V¹ is CR^{W}, CHR^{W}, or NR^{W}; provided that there are no O—O, S—S, or S—O bonds;
R¹ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{c1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{c1} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₃₋₁₀ cycloalkyl, 6-10 membered aryl, 4-membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆haloalkoxy, C₂₋₆ alkenyl, C₂₋₆ alkynyl, C₃₋₁₀ cycloalkyl, 6-10 membered aryl, 4-membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{c11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{c11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a1}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}) C(O)OR^{a12}OC(O)R^{b12}, OC(O)NR^{c12}R^{d12}, NR^{c12}R^{d12}, NR^{c12}NR^{c12}R^{d12}, NR^{c12}C(O)R^{b12} NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12} NR^{c12}S(O)₂R^{b12}, NR¹²S(O)(═NR^{c12})R^{b12}, NR^{c12}S(O)₂NR^{c12}R^{d12}, S(O)R^{b12} S(O)NR^{c12}R^{d12}, S(O)₂R^{b12} S(O)₂NR^{c12}R^{d12}, OS(O)(═NR^{c12})R^{b12} OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{e12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{c12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR²S(O)(═NR^{c2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{c2})R^{b2}, OS(O)₂R^{b2}, S(O)(═NR^{c2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{c21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{c21})R^{b21}, OS(O)₂R^{b21}, S(O)(═NR^{c21})R^{b21}, SF₅, P(O)R^{f1}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f1} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{a22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{c22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{c22})R^{b22}, OS(O)₂R^{b22}, S(O)(═NR^{c22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{e22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{e22} and R^{d22} attached to the same N atom, together with the N atom to which they are attached, form a 4-7 membered heterocycloalkyl group, wherein the 4-7 membered s heterocycloalkyl group is optionally substituted with 1, 2, 3, or 4 independently selected RG substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₃R^{b3}, NR^{c3}S(O)(═NR^{c3})R^{b3}, NR^{c3}S(O)₂NR^{c3}R^{d3}, S(O)R^{b3}, S(O)NR^{c3}R^{d3}, S(O)₂R^{b3}, S(O)₃NR^{c3}R^{d3}, OS(O)(═NR^{c3})R^{b3}, OS(O)₂R^{b3}, S(O)(═NR^{c3})R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁴ and R⁵ are each independently selected from H, C₁₋₆ alkyl, C₁₋₆ haloalkyl, cyano-C₁₋₃ alkyl, HO—C₁₋₃ alkyl, and C₁₋₃ alkoxy-C₁₋₃ alkyl; or
any R⁴ and R⁵ are, together with the carbon atom to which they are attached, form a 3-7-membered cycloalkyl ring;
R⁶ is independently selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, C(O)R^{b6}, C(O)NR^{e6}R^{d6}, C(O)NR^{c6}(OR^{a6}), C(O)OR^{a6}, C(═NR^{e6})R^{b6}, C(═NR^{e6})NR^{c6}R^{d6}, S(O)R^{b6}, S(O)NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6A} substituents;
each R^{c6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{6A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{c61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{c61})R^{b61}, OS(O)₂R^{b61}, S(O)(═NR^{c61})R^{b61}, SF₅, P(O)R^{f1}R^{g61}, OP(O)(OR^{h61})(ORⁱ⁶¹), P(O)(OR^{h61})(ORⁱ⁶¹), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered s heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR⁶²C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is:

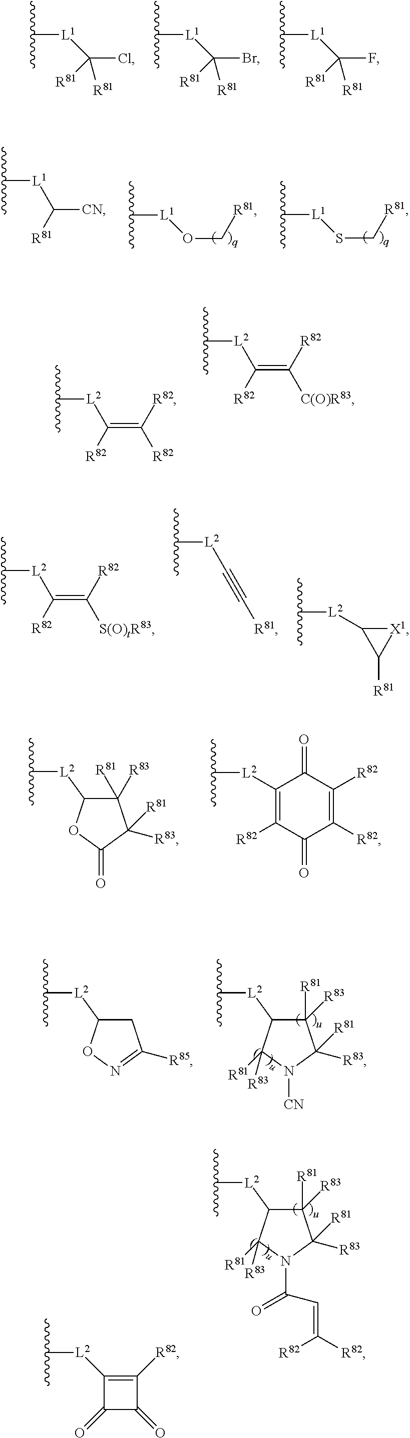

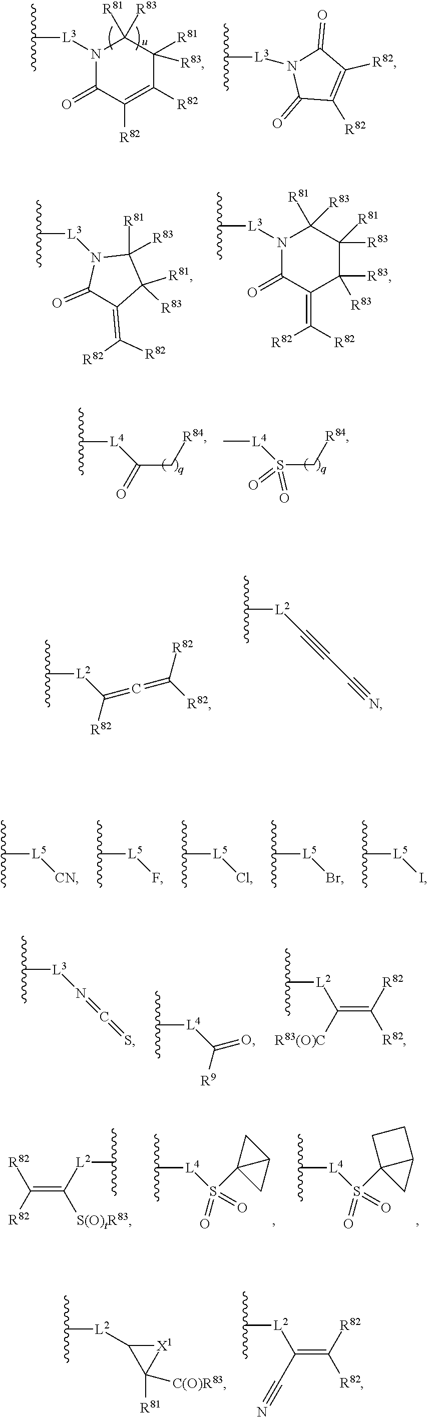

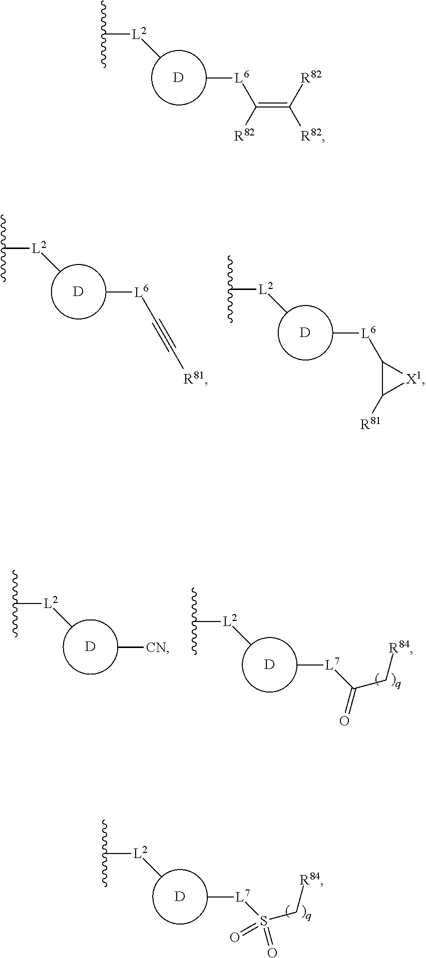

or L¹-Ar;
   L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Ring moiety A through the L linking group;
   L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Ring moiety A through the L linking group;
   L³ is -L-, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L³ is attached to Ring moiety A through the L linking group;
   L⁴ is -L-, -L-O—, L-S—, or -L-NR⁹—, wherein L⁴ is attached to Ring moiety A through the L linking group;
   L⁵ is -L-O-L^{x}-, -L-NR⁹-L^{x}-, -L-S-L^{x}-, -L-C(O)-L^{x}-, -L-NR⁹C(O)-L^{x}-, -L-OC(O)-L^{x}-, -L-S(O)-L^{x}-, -L-S(O)₂-L^{x}-, -L-NR⁹S(O)-L^{x}-, -L-OS(O)-L^{x}-, -L-NR⁹S(O)NR⁹-L^{x}-, -L-NR⁹S(O)O-L^{x}-, -L-OS(O)NR⁹-L^{x}-, -L-NR⁹S(O)₂-L^{x}-, -L-OS(O)₂-L^{x}-, -L-NR⁹S(O)₂NR⁹-L^{x}-, -L-NR⁹S(O)₂O-L^{x}-, -L-S(O)(NR⁹)-L^{x}-, -L-S(O)₂(NR⁹)-L^{x}-, or -L-OS(O)₂NR⁹-L^{x}-, wherein L⁵ is attached to Ring moiety A through the L linking group;
   L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—;
   L⁷ is -L-, -L-O—, L-S—,or -L-NR⁹—;
   each L is independently a bond or C₁₋₆ alkylene, wherein said C₁₋₆ alkylene is optionally substituted by 1, 2, 3 or 4 independently selected R^{G} substituents; or
   each L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
   each R^{N} is independently H or C₁₋₆ alkyl;
   each L^{x} is independently a C₁₋₆ alkylene, wherein said C₁₋₆ alkylene is optionally substituted by 1, 2, 3 or 4 independently selected R^{G} substituents;
   Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
   X¹ is O or NR⁹;
   each q is independently 0, 1, 2, or 3;
   each t is independently 0, 1, 2, or 3;
   each u is, independently, 0, 1, 2, or 3;
   Ar is C₆₋₁₀ aryl or 5-10 membered heteroaryl, which are each optionally substituted with 1, 2, 3, or 4 independently selected R⁹ substituents;
   each R⁸¹, and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
   each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 s membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{b8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
   each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
   each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
   each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
   each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
   each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
   each R^{j8} and R^{k8} is independently selected from OH, C₁₋₆ alkoxy, and C₁₋₆ haloalkoxy;
   or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
   or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substitutents;
   each R⁸⁴ is independently H, D, halo, CN, OH, C₁₋₃ alkoxy, C₁₋₃ haloalkoxy, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₃ alkoxy, C₁₋₃haloalkoxy, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are optionally substituted by 1, 2, 3, or 4 independently selected R^{G} substituents;
   each R⁸⁵ is independently H, D, halo, CN, C(O)H, OH, C₁₋₃ alkoxy, C₁₋₃ haloalkoxy, amino, C₁₋₆ alkyl, C₁₋₆ haloalkyl, C₃₋₇ cycloalkyl, 4-7 membered heterocycloalkyl, or C₃₋₇ cycloalkyl-C₁₋₄ alkyl, wherein said C₁₋₆ alkyl, C₁₋₆ haloalkyl, C₃₋₇ cycloalkyl, 4-7 membered heterocycloalkyl, and C₃₋₇ cycloalkyl-C₁₋₄ alkyl are each optionally substituted by 1, 2, 3, or 4 independently selected R^{G} substituents;
   each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{c9}, C(O)NR^{c9}R^{d9}, C(O)NR^{c9}(OR^{a9}), C(O)OR^{a9}, OC(O)R^{c9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{c9})R^{b9}, NR^{c9}S(O)₂NR^{c9}R^{d9}, S(O)R^{b9}, S(O)NR^{a9}R^{d9}, S(O)₂R^{b9}, S(O)₂NR^{a9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
   each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
   each R^{b9} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
   each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; and
   each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, when Ring A is Formula (A-1), then Y is CR^{W}, CHR^{W}, or NR^{W}; and Y¹ is CH, CH₂, N, or NH;
when Ring A is Formula (A-2), then Y is C, CH, or N; Y¹ is C, CH, or N; V is CR^{W}, CHR^{W}, or NR^{W}; and V¹ is CH, CH₂, N, or NH;
when Ring A is Formula (A-3), then Y is C; V is CR^{W}, CHR^{W}, or NR^{W}; and V¹ is CH, CH₂, N, or NH; and
when Ring A is Formula (A-4), then (i) Y is CH, CH₂, N, or NH; Y¹ is C; V is CR^{W}, CHR^{W}, or NR^{W}; and V¹ is CH, CH₂, N, or NH; or (ii) Y is CR^{W}, CHR^{W}, or NR^{W}; Y¹ is C; V is CH, CH₂, N, or NH; and V¹ is CH, CH₂, N, or NH.

In some embodiments, Ring moiety A is selected from Formulas (A-1) and (A-2):

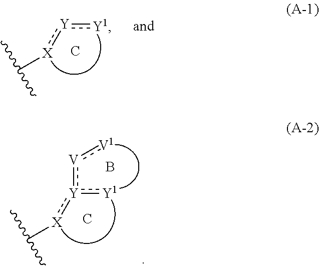

In some embodiments, Ring moiety A is selected from Formulas (A-1) and (A-2):

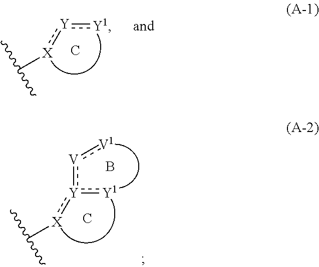

and
wherein when Ring A is Formula (A-1), then Y is CR^{W}, CHR^{W}, or NR^{W}; and Y¹ is CH, CH₂, N, or NH; and when Ring A is Formula (A-2), then Y is C, CH, or N; Y¹ is C, CH, or N; V is CR^{W}, CHR^{W}, or NR^{W}; and V¹ is CH, CH₂, N, or NH.

In some embodiments, Ring moiety A is Formula (A-1):

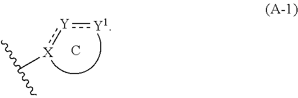

In some embodiments, Ring moiety A is Formula (A-1):

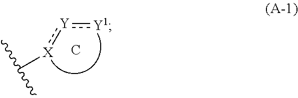

and
Y is CR^{W}, CHR^{W}, or NR^{W}; and Y¹ is CH, CH₂, N, or NH.

In some embodiments, Y is CR^{W}; and Y¹ is CH.

In some embodiments, Y is CR^{W}; and Y¹ is CH.

In some embodiments, Ring moiety A is Formula (A-2):

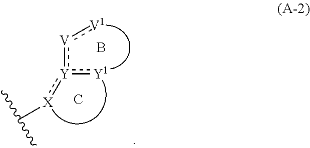

In some embodiments, Ring moiety A is Formula (A-2):

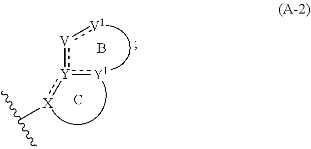

and
Y is C, CH, or N; Y¹ is C, CH, or N; V is CR^{W}, CHR^{W}, or NR^{W}; and V¹ is CH, CH₂, N, or NH.

In some embodiments, Y is C; Y¹ is C; V is NR^{W}; and V¹ is CH₂. In some embodiments, Ring B is 5-6-membered heteroaryl or 5-6 membered heterocycloalkyl, each of which is optionally substituted by 1, 2, 3, or 4 independently selected R² substituents. In some embodiments, Ring B is 5-6 membered heterocycloalkyl, which is optionally substituted by 1, 2, 3, or 4 independently selected R² substituents. In some embodiments, Ring B is 5-membered heterocycloalkyl, which is optionally substituted by 1, 2, 3, or 4 independently selected R² substituents. In some embodiments, Ring B is pyrrolidinyl.

In some embodiments, Ring C is phenyl, 5-6-membered heteroaryl, or 5-6-membered heterocycloalkyl, each of which is each optionally substituted by 1, 2, 3, or 4 independently selected R² substituents. In some embodiments, Ring C is phenyl, 5-6-membered heteroaryl, or 6-membered heterocycloalkyl, which is optionally substituted by 1, 2, 3, or 4 independently selected R² substituents. In some embodiments, Ring C is phenyl, 5-membered heteroaryl, or 6-membered heterocycloalkyl, which is optionally substituted by 1, 2, 3, or 4 independently selected R² substituents. In some embodiments, Ring C is phenyl or 6-membered heterocycloalkyl, each of which is optionally substituted by 1, 2, 3, or 4 independently selected R² substituents. In some embodiments, Ring C is phenyl or 1,2,3,4-tetrahydropyridin-6-yl, which is optionally substituted by 1, 2, 3, or 4 independently selected R² substituents. In some embodiments, Ring C is phenyl, which is optionally substituted by 1, 2, 3, or 4 independently selected R² substituents. In some embodiments, Ring C is 1,2,3,4-tetrahydropyridin-6-yl.

In some embodiments, Ring A is selected from,

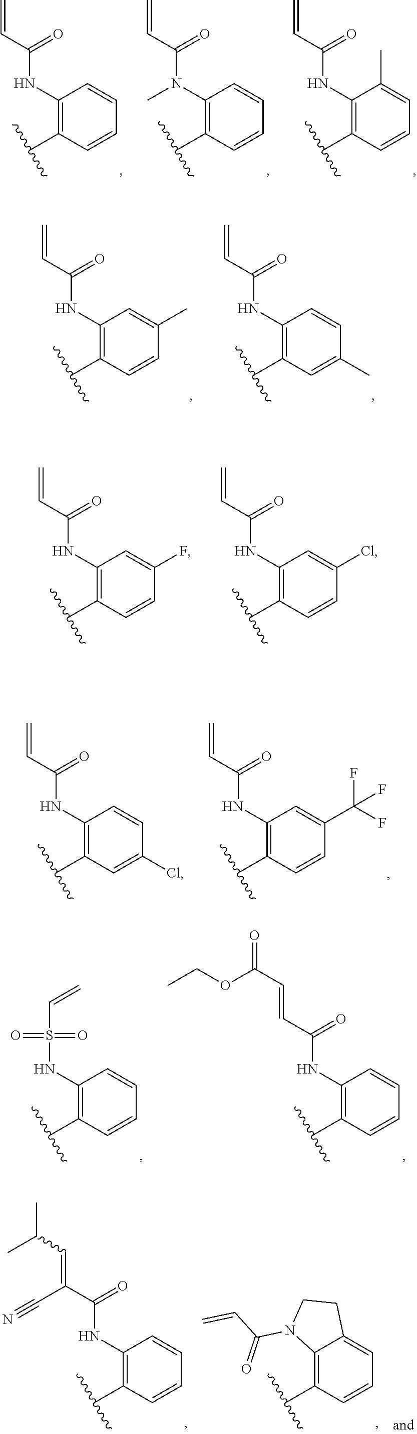

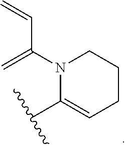 [SMILES: *C1=CCCCN1C(=C)C=C]

In some embodiments, Ring A

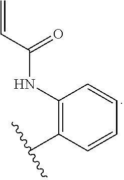 [SMILES: *c1ccccc1NC(=O)C=C]

In some embodiments, Ring A is

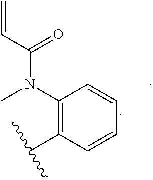 [SMILES: *c1ccccc1N(C)C(=O)C=C]

In some embodiments, Ring A is

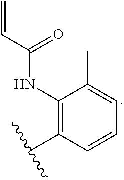 [SMILES: *c1cccc(C)c1NC(=O)C=C]

In some embodiments, Ring A is

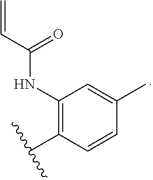 [SMILES: *c1ccc(C)cc1NC(=O)C=C]

In some embodiments, Ring A is

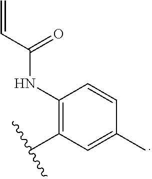 [SMILES: *c1cc(C)ccc1NC(=O)C=C]

In some embodiments, Ring A is

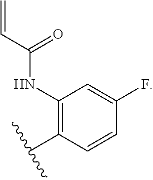 [SMILES: *c1ccc(F)cc1NC(=O)C=C]

In some embodiments, Ring A is

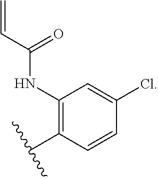 [SMILES: *c1ccc(Cl)cc1NC(=O)C=C]

In some embodiments, Ring A is

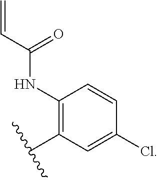 [SMILES: *c1cc(Cl)ccc1NC(=O)C=C]

In some embodiments, Ring A is

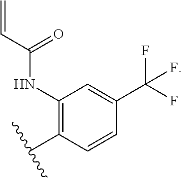 [SMILES: *c1ccc(C(F)(F)F)cc1NC(=O)C=C]

In some embodiments, Ring A is

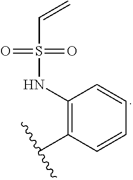 [SMILES: *c1ccccc1NS(=O)(=O)C=C]

In some embodiments, Ring A is

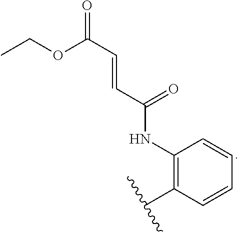 [SMILES: *c1ccccc1NC(=O)/C=C/C(=O)OCC]

In some embodiments, Ring A is

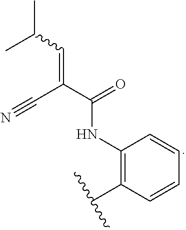 [SMILES: *c1ccccc1NC(=O)C(C#N)=CC(C)C]

In some embodiments, Ring A is

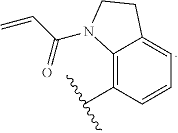 [SMILES: *c1cccc2c1N(C(=O)C=C)CC2]

In some embodiments, Ring A is

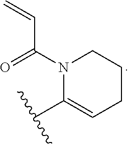 [SMILES: *C1=CCCCN1C(=O)C=C]

In some embodiments, X is C, CH, or N. In some embodiments, X is C or CH. In some embodiments, X is C.

In some embodiments, R¹ is independently selected from H, D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a1}, SR^{a1}, NHOR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents.

In some embodiments, R¹ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a1}, SR^{a1}NHOR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

In some embodiments, R¹ is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a1}, SR^{a1}, NHOR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected RA substituents.

In some embodiments, R¹ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected RA substituents.

In some embodiments, R¹ is independently selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl, each of which is optionally substituted with 1, 2, 3, or 4 independently selected R^{1A} substituents.

In some embodiments, R¹ is independently selected from C₁₋₆ alkyl, C₂₋₆ alkenyl, C₂₋₆ alkynyl, C₃₋₇ cycloalkyl, phenyl, 4-7 membered heterocycloalkyl, and 5-6 membered heteroaryl, each of which are each optionally substituted with 1, 2, 3, or 4 independently selected RA substituents.

In some embodiments, R¹ is independently selected from C₁₋₆ alkyl, C₃₋₁₀ cycloalkyl, 6-membered heterocycloalkyl, and 5-membered heteroaryl, wherein the 6-membered heterocycloalkyl is optionally substituted with R^{1A}. In some embodiments, R¹ is independently selected from C₁₋₆ alkyl, C₃₋₁₀ cycloalkyl, and 6-membered heterocycloalkyl, wherein the 6-membered heterocycloalkyl is optionally substituted with C(O)C₂₋₆ alkenyl. In some embodiments, R^{1A} is independently selected from C(O)R^{b11}, C(O)NR^{c11}R^{d11}, C(O)OR^{a11}, OC(O)R^{b11}, and NR^{c11}C(O)R^{b11}. In some embodiments, R^{1A} is C(O)R^{b11}.

In some embodiments:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents; and
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents.

In some embodiments:
each R^{a1}, R^{c1}, and R^{d1} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1A} substituents; and
each R^{b1} is independently selected from C₁₋₆ alkyl, C₂₋₆ alkenyl, C₂₋₆ alkynyl, C₁₋₆ haloalkyl, and C₃₋₄ cycloalkyl, which are each optionally substituted with 1, 2, 3, or 4 independently selected R^{1A} substituents.

In some embodiments, each R^{a11}, R^{b11}, R¹¹, and R^{d11} is selected from C₁₋₆ alkyl, C₂₋₆ alkenyl, and C₂₋₆ alkynyl. In some embodiments, each R^{a11}, R^{b11}, R^{c11}, and R^{d11} is independently C₂₋₆ alkenyl.

In some embodiments:
each R^{1A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a1}, SR^{a11}, NHOR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents; and
each R^{1B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{1A} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{a11}, R^{c11}, and R^{d11} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, and C₃₋₄ cycloalkyl, which are each optionally substituted with 1, 2, 3, or 4 independently selected R^{1B} substituents; and
each R^{1B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R^{1A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R¹ is independently selected from methyl, isopropyl, cyclopropyl, cyclobutyl, cyclohexyl, 3,4-dihydropyran, tetrahydropyran, piperidine, and thiophene, wherein the piperidine is optionally substituted with C(O)C₂ alkenyl. In some embodiments, R¹ is independently selected from methyl, isopropyl, cyclopropyl, cyclobutyl, cyclohexyl,

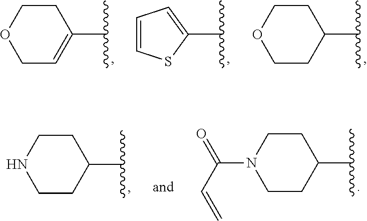

In some embodiments, R¹ is methyl. In some embodiments, R is isopropyl. In some embodiments, R¹ is cyclopropyl. In some embodiments, R¹ is cyclobutyl. In some embodiments, R¹ is cyclohexyl. In some embodiments, R¹ is

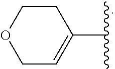 [SMILES: *C1=CCOCC1]

In some embodiments, R¹ is

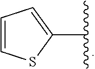 [SMILES: *c1cccs1]

In some embodiments, R¹ is

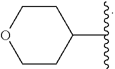 [SMILES: *C1CCOCC1]

In some embodiments, R¹ is

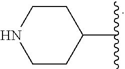 [SMILES: *C1CCNCC1]

In some embodiments, R¹ is

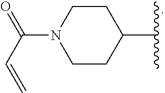 [SMILES: *C1CCN(C(=O)C=C)CC1]

In some embodiments, R² is independently selected from H, D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{e2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents.

In some embodiments, R² is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents.

In some embodiments, R² is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a2}, SR^{a2}, NHOR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{2A} substituents.

In some embodiments, R² is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a2}, SR^{a2}, NHOR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR²S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents.

In some embodiments:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents; and
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents.

In some embodiments:
each R^{a2}, R^{c2}, and R^{d2} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1A} substituents; and
each R^{b2} is independently selected from C₁₋₆ alkyl, C₂₋₆ alkenyl, C₂₋₆ alkynyl, C₁₋₆ haloalkyl, and C₃₋₄ cycloalkyl, which are each optionally substituted with 1, 2, 3, or 4 independently selected R^{2A} substituents.

In some embodiments, each R^{a2}, R^{c2}, and R^{d2} is independently selected from H, C₁₋₆ alkyl, C₂₋₆ alkenyl, C₂₋₆ alkynyl, and C₁₋₆ haloalkyl; and each R^{b2} is independently selected from C₁₋₆ alkyl, C₂₋₆ alkenyl, C₂₋₆ alkynyl, and C₁₋₆ haloalkyl.

In some embodiments, R² is independently selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments:
each R^{2A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents; and
each R^{2B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{2A} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a21}, C(O)R^{b21}, C(O)NR^{e21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{a21}, R^{c21}, and R^{d21} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, and C₃₋₄ cycloalkyl, which are each optionally substituted with 1, 2, 3, or 4 independently selected R^{2B} substituents; and
each R^{2B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R^{2A} is independently selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, each R^{2A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R² is selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R² is selected from D, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₃₋₇ cycloalkyl, C₁₋₆ alkoxy, and C₁₋₃ haloalkoxy.

In some embodiments, R² is selected from halo, C₁₋₆ alkyl, and C₁₋₆ haloalkyl. In some embodiments, R² is selected from F, Cl, CH₃, and CF₃. In some embodiments, R² is Cl. In some embodiments, R² is CH₃. In some embodiments, R² is F. In some embodiments, R² is CF₃.

In some embodiments, R³ is independently selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R³ is selected from D, OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino. In some embodiments, R³ is selected from C₁₋₆ alkyl, C₂₋₆ alkenyl, C₂₋₆ alkynyl, C₁₋₆ haloalkyl, cyano-C₁₋₆ alkyl, HO—C₁₋₆ alkyl, and C₁₋₆ alkoxy-C₁₋₆ alkyl. In some embodiments, R³ is C₁₋₆ alkyl. In some embodiments, R³ is —CH₂CH₃.

In some embodiments, R⁴ and R⁵ are each independently selected from H, and C₁₋₆ alkyl; or R⁴ and R⁵, together with the carbon atom to which they are attached, form a 3-7-membered cycloalkyl ring.

In some embodiments, R⁴ and R⁵ are independently selected from H, and C₁₋₆ alkyl. In some embodiments R⁴ and R⁵, together with the carbon atom to which they are attached, form a 3-7-membered cycloalkyl ring. In some embodiments, R⁴ and R⁵ are each independently H or CH₃. In some embodiments, R⁴ and R⁵ are each H.

In some embodiments, any R⁴ and R⁵ are, together with the carbon atom to which they are attached, form a 3-7-membered cycloalkyl ring.

In some embodiments, R⁶ is independently selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, each of which is optionally substituted by 1, 2, 3 or 4 independently selected R^{6A} substituents.

In some embodiments, R⁶ is independently selected from C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, each of which is optionally substituted by 1, 2, 3 or 4 independently selected R^{6A} substituents.

In some embodiments, R⁶ is independently selected from C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, each of which is optionally substituted by 1, 2, 3 or 4 independently selected R^{6A} substituents.

In some embodiments, R⁶ is independently selected from 6-10 membered aryl, or 5-10 membered heteroaryl, each of which is optionally substituted by 1, 2, 3 or 4 independently selected R^{6A} substituents.

In some embodiments, R⁶ is independently selected from phenyl or 5-6 membered heteroaryl, each of which is optionally substituted by 1, 2, 3 or 4 independently selected R^{6A} substituents. In some embodiments, R⁶ is phenyl which is optionally substituted by 1 or 2 independently selected R^{6A} substituents. In some embodiments, R⁶ is phenyl which is substituted by 1 or 2 independently selected R^{6A} substituents.

In some embodiments:
each R^{6A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents; and
each R^{6B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{6A} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61} NR^{c61}S(O)₂R^{b61} NR^{c61}S(O)₂NR^{c61}R^{d61}, S(O)₂R^{b61} and S(O)₂NR^{c61}R^{d61}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{6B} substituents;
each R^{a61}, R^{c61}, and R^{d61} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, and C₃₋₄ cycloalkyl, which are each optionally substituted with 1, 2, 3, or 4 independently selected R^{6B} substituents; and
each R^{6B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R^{6A} is independently selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, each R^{6A} is independently selected from D, OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino. In some embodiments, each R^{6A} is independently selected from OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino.

In some embodiments, each R^{6A} is independently selected from halo, C₁₋₆ alkyl, and C₁₋₆ haloalkyl. In some embodiments, each R^{6A} is independently selected from halo and C₁₋₆ haloalkyl. In some embodiments, each R^{6A} is independently selected from Cl and CF₃. In some embodiments, R^{6A} is Cl. In some embodiments, R^{6A} is CF₃.

In some embodiments, R⁶ is

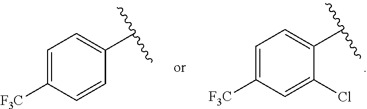 [SMILES: *c1ccc(C(F)(F)F)cc1 ; *c1ccc(C(F)(F)F)cc1Cl]

In some embodiments, R⁶ is

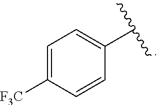 [SMILES: *c1ccc(C(F)(F)F)cc1]

In some embodiments, R⁶ is

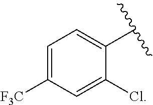 [SMILES: *c1ccc(C(F)(F)F)cc1Cl]

In some embodiments, R^{W} is a covalent warhead that covalently binds to a cysteine of WRN. In some embodiments, R^{W} is a covalent warhead that covalently binds to cysteine-727 of WRN.

In some embodiments, R^{W} is selected from:

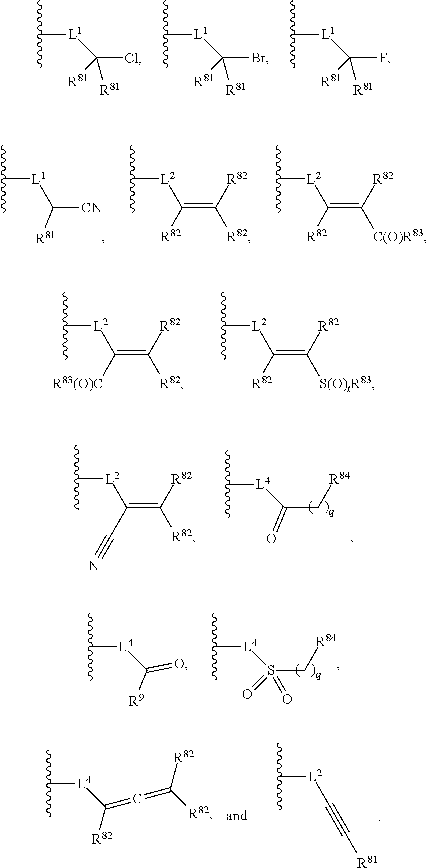

In some embodiments R^{W} is selected from

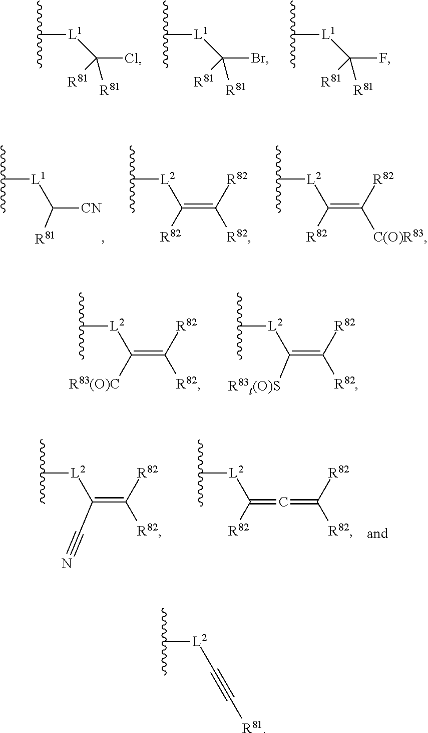

In some embodiments, R^{W} is selected from:

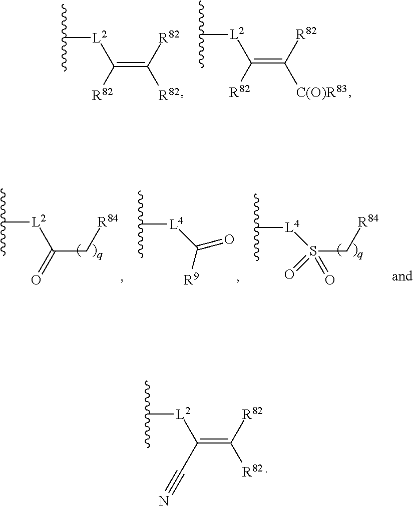

In some embodiments, R^{W} is

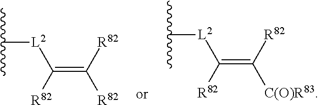

In some embodiments, R^{W} is

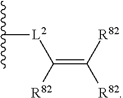

In some embodiments, R^{W} is

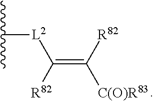

In some embodiments:
each R⁸¹ and R⁸³ is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OW^{a8}, SR^{a8}, C(O)R^{b8}, C(O)NR^{c8}R^{d8}, C(O)OR^{a8}, OC(O)R^{b8}, OC(O)NR^{c8}R^{d8}, NR^{c8}R^{d8}, NR^{c8}C(O)R^{b8}, NR^{c8}C(O)OR^{a8}, NR^{c8}C(O)NR^{c8}R^{d8}, NR^{c8}S(O)₂R^{b8}, NR^{c8}S(O)₂NR^{c8}R^{d8}, S(O)₂R^{b8}, and S(O)₂NR^{c8}R^{d8}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, S(O)₂R^{b8}, and S(O)₂NR^{c8}R^{d8}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{G} substituents; and
each R^{b8} is independently selected from C₁₋₆ alkyl and C₁₋₆ haloalkyl, which are each optionally substituted with 1, 2, 3, or 4 independently selected R^{G} substituents.

In some embodiments:
each R⁸¹ and R⁸³ is independently selected from H, D, halo, CN, C₁₋₆ alkyl, C₁₋₆ haloalkyl, C₃₋₄ cycloalkyl, and OR^{a8}; wherein said C₁₋₆ alkyl, C₁₋₆ haloalkyl, and C₃₋₄ cycloalkyl are each optionally substituted by 1, 2, 3, or 4 independently selected R^{G} substituents;
each R⁸² is independently selected from H, D, halo, CN, C₁₋₆ alkyl, C₁₋₆ haloalkyl, and C₃₋₄ cycloalkyl; wherein said C₁₋₆ alkyl, C₁₋₆ haloalkyl, and C₃₋₄ cycloalkyl are each optionally substituted by 1, 2, 3, or 4 independently selected R^{G} substituents; and
each R^{a8} is independently selected from C₁₋₆ alkyl, and C₁₋₆ haloalkyl.

In some embodiments:
each R⁸¹ and R⁸³ is independently selected from H, D, halo, CN, C₁₋₆ alkyl, C₁₋₆ haloalkyl, C₁₋₆ alkoxy, C₁₋₆ haloalkoxy, and C₃₋₄ cycloalkyl; wherein said C₁₋₆ alkyl, C₁₋₆ haloalkyl, C₁₋₆ alkoxy, C₁₋₆ haloalkoxy, and C₃₋₄ cycloalkyl are each optionally substituted by 1, 2, 3, or 4 independently selected R^{G} substituents; and
each R⁸² is independently selected from H, D, halo, CN, C₁₋₆ alkyl, C₁₋₆ haloalkyl, and C₃₋₄ cycloalkyl; wherein said C₁₋₆ alkyl, C₁₋₆ haloalkyl, and C₃₋₄ cycloalkyl are each optionally substituted by 1, 2, 3, or 4 independently selected R^{G} substituents.

In some embodiments, each R⁸¹ and R⁸³ is independently selected from H, D, halo, CN, C₁₋₆ alkyl, C₁₋₆ haloalkyl, C₁₋₆ alkoxy, C₁₋₆ haloalkoxy, and C₃₋₄ cycloalkyl; and each R⁸² is independently selected from H, D, halo, CN, C₁₋₆ alkyl, C₁₋₆ haloalkyl, and C₃₋₄ cycloalkyl.

In some embodiments:
each R⁸¹ and R⁸³ is independently selected from H, D, halo, CN, C₁₋₆ alkyl, C₂₋₆ alkenyl, C₂₋₆ alkynyl, C₁₋₆ haloalkyl, C₃₋₇ cycloalkyl, C₁₋₆ alkoxy, C(O)R^{b8}, C(O)NR^{c8}R^{d8}, and C(O)OR^{a8};
each R⁸² is independently selected from H, D, halo, CN, C₁₋₆ alkyl, C₂₋₆ alkenyl, C₂₋₆ alkynyl, C₁₋₆ haloalkyl, C₃₋₇ cycloalkyl, C(O)R^{b8}, C(O)NR^{c8}R^{d8}, and C(O)OR^{a8}; and
each R^{a8}, R^{c8}, and R^{d8} is independently selected from H, C₁₋₆ alkyl, and C₃₋₇ cycloalkyl.

In some embodiments, each R⁸¹ and R⁸³ is independently selected from H, CN, C₁₋₆ alkyl, and C₁₋₆ alkoxy; and each R⁸² is independently selected from H, CN, and C₁₋₆ alkyl.

In some embodiments, each R⁸¹ and R⁸³ is independently selected from H, methyl, ethyl, CN, isopropyl, and ethoxy; and each R⁸² is independently selected from H, methyl, ethyl, CN, and isopropyl.

In some embodiments, each R⁸² is independently selected from H, CN, and C₁₋₆ alkyl. In some embodiments, each R⁸² is independently selected from H, CN, and isopropyl. In some embodiments, R⁸² is H. In some embodiments, R⁸² is CN. In some embodiments, R⁸² is isopropyl. In some embodiments, R⁸³ is C₁₋₆ alkoxy. In some embodiments, each R⁸³ is ethoxy.

In some embodiments, R⁹ is selected from H, halo, C₁₋₆ alkyl, C₂₋₆ alkenyl, C₂₋₆ alkynyl, C₁₋₆ haloalkyl, and C₃₋₇ cycloalkyl.

In some embodiments, R^{W} is selected from

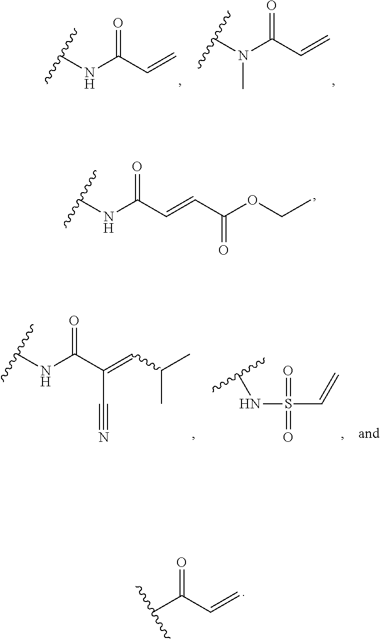

In some embodiments, L¹ is -L-C(O)—, -L-NR⁹C(O)—, -L-S(O)₂—, or -L-NR⁹S(O)—, wherein L¹ is attached to Ring moiety A through an L linking group.

In some embodiments, L² is -L-C(O)—, -L-NR⁹C(O)—, -L-S(O)₂—, or -L-NR⁹S(O)—, wherein L² is attached to Ring moiety A through an L linking group.

In some embodiments, L² is -L-C(O)—, -L-NR⁹C(O)- or -L-NR⁹S(O)₂—, wherein L² is attached to Ring moiety A through an L linking group. In some embodiments, L² is -L-C(O)-.

In some embodiments, L² is -L-NR⁹C(O)-. In some embodiments, L² is -L-NR⁹S(O)₂—.

In some embodiments, L⁴ is -L-O—, or -L-NR⁹—, wherein L⁴ is attached to Ring moiety A through the L linking group. In some embodiments, L⁴ is -L-NR⁹—.

In some embodiments, each L is independently a bond or C₁₋₆ alkylene, wherein said C₁₋₆ alkylene is optionally substituted by 1, 2, 3 or 4 independently selected R^{G} substituents.

In some embodiments, each L is independently a bond or C₁₋₆ alkylene.

In some embodiments, each L is a bond.

In some embodiments, R⁹ is selected from H, halo, C₁₋₆ alkyl, C₂₋₆ alkenyl, C₂₋₆ alkynyl, C₁₋₆ haloalkyl, C₃₋₇ cycloalkyl. In some embodiments, R⁹ is H or C₁₋₆ alkyl. In some embodiments, R⁹ is H or methyl. In some embodiments, R⁹ is H. In some embodiments, R⁹ is methyl.

In some embodiments:
Ring A is selected from Formula

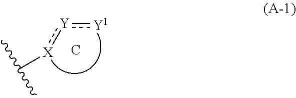

### and Formula

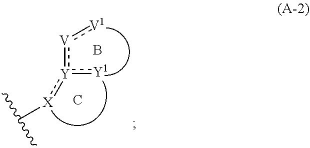

when Ring A is Formula (A-1), then (i) Y is CR^{W}, CHR^{W}, or NR^{W}; and Y¹ is CH, CH₂, N, NH, S or O; or (ii) Y is CH, CH₂, N, NH, S or O; and Y¹ is CR^{W}, CHR^{W}, or NR^{W}; provided that there are no O—O, S—S, or S—O bonds;
when Ring A is Formula (A-2), then (i) Y is C; Y¹ is C; V is CR^{W}, CHR^{W}, or NR^{W}; and V¹ is CH, CH₂, N, NH, S or O; or (ii) Y is C; Y¹ is C; V is CH, CH₂, N, NH, S or O; and V¹ is CR^{W}, CHR^{W}, or NR^{W}; provided that there are no O—O, S—S, or S—O bonds;
X is C, CH, or N;
Ring B is 5-6 membered heterocycloalkyl, which is optionally substituted by 1, 2, 3, or 4 independently selected R² substituents.
Ring C is phenyl, 5-6-membered heteroaryl, or 5-6-membered heterocycloalkyl, wherein phenyl, 5-6-membered heteroaryl, and 6-membered heterocycloalkyl is each optionally substituted by 1, 2, 3, or 4 independently selected R² substituents;
R¹ is independently selected from C₁₋₆ alkyl, C₂₋₆ alkenyl, C₂₋₆ alkynyl, C₃₋₁₀ cycloalkyl, 6-membered aryl, 4-7 membered heterocycloalkyl, and 5-6 membered heteroaryl, wherein said C₁₋₆ alkyl, C₂₋₆ alkenyl, C₂₋₆ alkynyl, C₃₋₁₀ cycloalkyl, 6-membered aryl, 4-7 membered heterocycloalkyl, 5-6 membered heteroaryl, are each optionally substituted with 1, 2, 3, or 4 independently selected R^{1A} substituents;
each R^{1A} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{a11}, R^{c11}, and R^{d11} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, and C₃₋₄ cycloalkyl, which are each optionally substituted with 1, 2, 3, or 4 independently selected R^{1B} substituents;
each R^{1B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is selected from D, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₃₋₇ cycloalkyl, C₁₋₆ alkoxy, and C₁₋₃ haloalkoxy;
R³ is selected from C₁₋₆ alkyl, C₂₋₆ alkenyl, C₂₋₆ alkynyl, C₁₋₆ haloalkyl, cyano-C₁₋₆ alkyl, HO—C₁₋₆ alkyl, and C₁₋₆ alkoxy-C₁₋₆ alkyl;
R⁴ and R⁵ are independently selected from H, and C₁₋₆ alkyl;
or R⁴ and R⁵, together with the carbon atom to which they are attached, form a 3-7-membered cycloalkyl ring;
R⁶ is independently selected from phenyl, or 5-6 membered heteroaryl, each of which is optionally substituted by 1, 2, 3 or 4 independently selected R^{6A} substituents;
each R^{6A} is independently selected from OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino;
R^{W} is selected from:

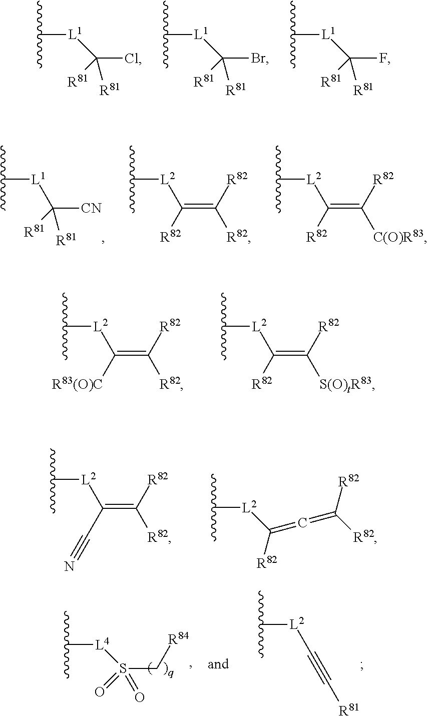

L¹ is -L-C(O)—, -L-NR⁹C(O)—, -L-OC(O)—, -L-NR⁹S(O)—, or -L-NR⁹S(O)₂—, wherein L¹ is attached to Ring moiety A through the L linking group;
L² is -L-C(O)—, -L-NR⁹C(O)- or -L-NR⁹S(O)₂—, wherein L⁴ is attached to Ring moiety A through the L linking group;
L⁴ is -L-, -L-O—, or -L-NR⁹—, wherein L⁴ is attached to Ring moiety A through the L linking group;
each R⁸¹, R⁸², and R⁸³ is independently selected from H, D, halo, CN, C₁₋₆ alkyl, C₂₋₆ alkenyl, C₂₋₆ alkynyl, C₁₋₆ haloalkyl, C₃₋₇ cycloalkyl, and C₁₋₆ alkoxy, C(O)R^{b8}, C(O)NR^{c8}R^{d8}, and C(O)OR^{a8};
each R^{a8}, R^{c8}, and R^{d8} is independently selected from H, C₁₋₆ alkyl, and C₃₋₇ cycloalkyl; and
R⁹ is selected from H, halo, C₁₋₆ alkyl, C₂₋₆ alkenyl, C₂₋₆ alkynyl, C₁₋₆ haloalkyl, C₃₋₇ cycloalkyl.

In some embodiments:
Ring A is selected from Formula (A-1) and (A-2):

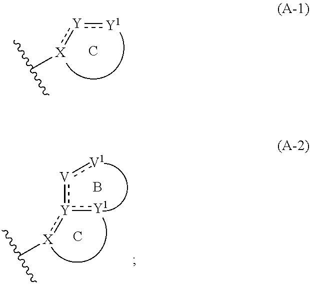

when Ring A is Formula (A-1), then (i) Y is CR^{W}, CHR^{W}, or NR^{W}; and Y¹ is CH, CH₂, N, or NH; or (ii) Y is CH, CH₂, N, or NH; and Y¹ is CR^{W}, CHR^{W}, or NR^{W}; or
when Ring A is Formula (A-2), then (i) Y is C; Y¹ is C; V is CR^{W}, CHR^{W}, or NR^{W}; and V¹ is CH, CH₂, N, or NH; or (ii) Y is C; Y¹ is C; V is CH, CH₂, N, or NH; and V¹ is CR^{W}, CHR^{W}, or NR^{W};
X is C or CH;
Ring B is 5-6-membered heteroaryl or 5-6 membered heterocycloalkyl, each of which is optionally substituted by 1, 2, 3, or 4 independently selected R² substituents;
Ring C is phenyl, 5-6-membered heteroaryl, or 5-6-membered heterocycloalkyl, each of which is each optionally substituted by 1, 2, 3, or 4 independently selected R² substituents;
R¹ is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, each of which are each optionally substituted with 1, 2, 3, or 4 independently selected R^{1A} substituents;
each R^{1A} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11} and S(O)₂NR^{c11}R^{d11}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{a11}, R^{c11}, and R^{d11} is independently selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, and C₃₋₄ cycloalkyl, which are each optionally substituted with 1, 2, 3, or 4 independently selected R^{1B} substituents;
each R^{1B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a2}, SR^{a2}, NHOR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
each R^{a2}, R^{c2}, and R^{d2} is independently selected from H, C₁₋₆ alkyl, C₂₋₆ alkenyl, C₂₋₆ alkynyl, and C₁₋₆ haloalkyl;
each R^{b2} is independently selected from C₁₋₆ alkyl, C₂₋₆ alkenyl, C₂₋₆ alkynyl, and C₁₋₆ haloalkyl;
R³ is halo, CN, C₁₋₆ alkyl, and C₁₋₆ haloalkyl;
R⁴ and R⁵ are each independently H, C₁₋₆ alkyl, and C₁₋₆ haloalkyl;
R⁶ is C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, each of which is optionally substituted by 1, 2, 3 or 4 independently selected R^{6A} substituents;
each R^{6A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{c61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is

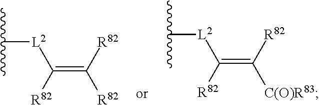

L² is -L-C(O)—, -L-NR⁹C(O)— or -L-NR⁹S(O)₂—, wherein L² is attached to Ring moiety A through the L linking group;
each L is a bond;
each R⁸¹ and R⁸³ is independently selected from H, D, halo, CN, C₁₋₆ alkyl, C₁₋₆ haloalkyl, C₃₋₄ cycloalkyl, and OR^{a8}; wherein said C₁₋₆ alkyl, C₁₋₆ haloalkyl, and C₃₋₄ cycloalkyl are each optionally substituted by 1, 2, 3, or 4 independently selected R^{G} substituents;
each R⁸² is independently selected from H, D, halo, CN, C₁₋₆ alkyl, C₁₋₆ haloalkyl, and C₃₋₄ cycloalkyl; wherein said C₁₋₆ alkyl, C₁₋₆ haloalkyl, and C₃₋₄ cycloalkyl are each optionally substituted by 1, 2, 3, or 4 independently selected R^{G} substituents;
each R^{a8} is independently selected from C₁₋₆ alkyl, and C₁₋₆ haloalkyl; and
R⁹ is H or C₁₋₆ alkyl.

In some embodiments:
Ring A is selected from Formulas (A-1) and (A-2):

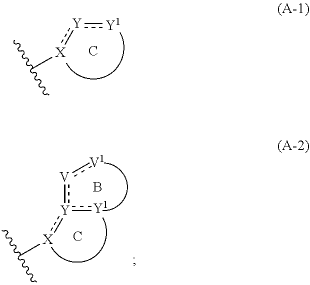

when Ring A is Formula (A-1), then Y is CR^{W}, or NR^{W}, and Y¹ is CH or CH₂; or
when Ring A is Formula (A-2), then Y is C, Y¹ is C, V is NR^{W}, and V¹ is CH₂;
X is C or CH;
Ring B is 5-membered heterocycloalkyl, which is optionally substituted by 1, 2, 3, or 4 independently selected R² substituents;
Ring C is phenyl or 6 membered heterocycloalkyl, which is optionally substituted by 1, 2, 3, or 4 independently selected R² substituents;
R¹ is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, each of which is optionally substituted with 1, 2, 3, or 4 independently selected R^{1A} substituents;
each R^{1A} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is selected from halo, CN, C₁₋₆ alkyl, and C₁₋₆ haloalkyl;
R³ is C₁₋₆ alkyl;
R⁴ and R⁵ are each H;
R⁶ is phenyl which optionally is substituted by 1, 2, 3, or 4 independently selected R^{6A} substituents;
each R^{6A} is independently selected from D, OH, NO₂, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₇ cycloalkyl, C₁₋₆ alkoxy, C₁₋₃ haloalkoxy, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonyloxy, C₁₋₆ alkylcarbonylamino, C₁₋₆ alkoxycarbonylamino, C₁₋₆ alkylaminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
R^{W} is

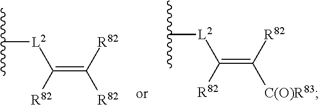

L² is -L-C(O)—, -L-NR⁹C(O)— or -L-NR⁹S(O)₂—;
each L is a bond;
each R⁸¹ and R⁸³ is independently selected from H, CN, C₁₋₆ alkyl, and C₁₋₆ alkoxy;
each R⁸² is independently selected from H, CN, and C₁₋₆ alkyl; and
R⁹ is H or methyl.

In some embodiments:
Ring A is selected from:

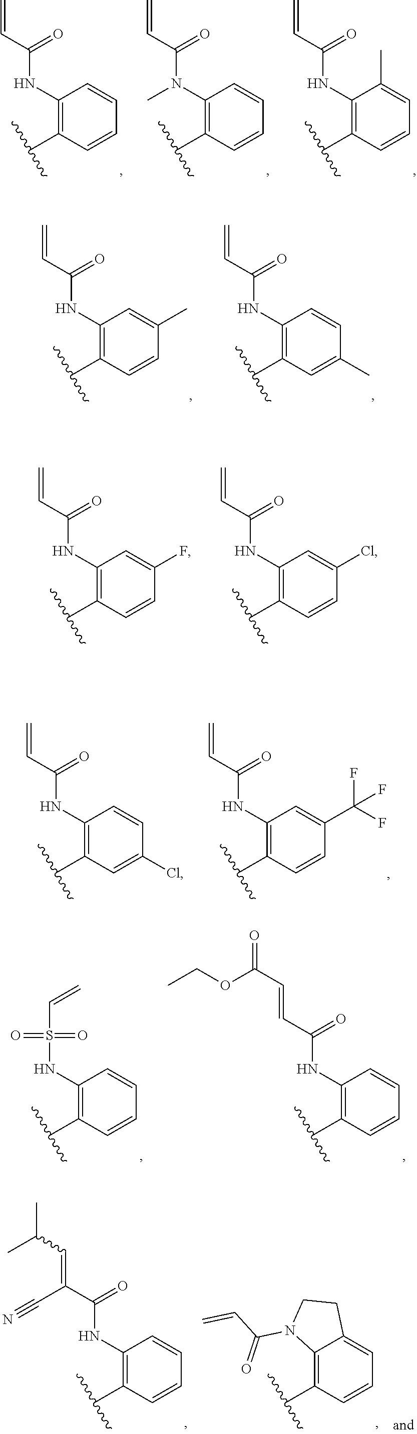

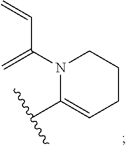 [SMILES: *C1=CCCCN1C(=C)C=C]

R¹ is independently selected from methyl, isopropyl, cyclopropyl, cyclobutyl, cyclohexyl,

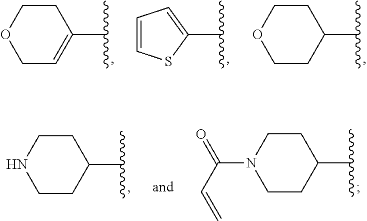

R² is selected from F, Cl, CH₃, and CF₃;
R³ is —CH₂CH₃;
R⁴ and R⁵ are each H; and
R⁶ is

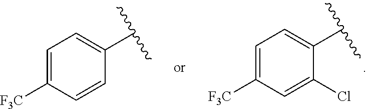 [SMILES: *c1ccc(C(F)(F)F)cc1 ; *c1ccc(C(F)(F)F)cc1Cl]

In some embodiments, compounds of Formula (I), or a pharmaceutically acceptable salt thereof, can include one or more hydrogen atoms replaced by deuterium. In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

In some embodiments, the substituent R² can replace any H atom on Ring moiety A, including on CH, CH₂, or NH moieties of X, Y, Y¹, Y², V, and V¹. However, the substituent R² cannot replace atoms or groups other than H, such as R^{W}.

It is further appreciated that certain features of the present disclosure, which are, for clarity, described in the context of separate embodiments, can also be provided in combination in a single embodiment (while the embodiments are intended to be combined as if written in multiply dependent form). Conversely, various features of the present disclosure which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, when an optionally multiple substituent is designated in the form:

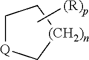

then it is to be understood that substituent R can occur p number of times on the ring, and R can be a different moiety at each occurrence. It is to be understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

Throughout the definitions, the term “Cn-m” indicates a range which includes the endpoints, wherein n and m are integers and indicate the number of carbons. Examples include C₁₋₃, C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cn-m alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cn-m alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cn-m alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cn-m aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms.

In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Co-p alkyl” refers to a group of formula —(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁₋₃ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH— alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O— alkyl, wherein the alkyl group has n to m carbon atoms.

As used herein, the term “carbamyl” to a group of formula —C(O)NH₂.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington 's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis of the Compounds

Compounds of the present disclosure, including salts thereof, can be prepared using known organic synthesis techniques and can be synthesized according to any of numerous possible synthetic routes.

Compounds of Formula (I) can be prepared as shown in Scheme 1. Condensation of aminotriazole 1-1, where X¹ is a halogen (e.g., Cl, Br, or I) or pseudohalogen (e.g., OTf or OMs), with dicarbonyl compound 1-2 at elevated temperatures (e.g., in the presence of acetic acid) can afford bicyclic 1-3. Heteroaryl 1-3 can be alkylated under standard S_{N}2 conditions (e.g., in the presence of diisopropylethylamine and 1-4, where X² is a halogen (e.g., Cl, Br, or I) or pseudohalogen (e.g., OTf or OMs)) to give compound 1-5. Compound 1-5 can be coupled with R¹-M¹ (1-6), where M¹ is a boronic acid, boronate ester, potassium trifluoroborate, or an appropriately substituted metal such as Sn(Bu)₃ or Zn, under standard Suzuki conditions (e.g., in the presence of a palladium catalyst, such as chloro(2-dicyclohexylphosphino-2′,4′,6′-triisopropyl-1,1′-biphenyl)[2-(2′-amino-1,1′-biphenyl)]palladium(II) and a base (e.g., a phosphate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(O) or [1,1′-bis(diphenylphosphino)ferrocene]dichloropalladium (II)), to give compound 1-7. Ester 1-7 can be hydrolyzed to give the corresponding carboxylic acid by either standard acidic conditions (e.g., in the presence of trifluoroacetic acid) or standard basic conditions (e.g., in the presence of lithium hydroxide and water). The acid can be converted to amide 1-9 by standard amide formation condition (e.g., in the presence of amine 1-8, a coupling reagent, such as hexafluorophosphate azabenzotriazole uronium (HATU) and a base, such as triethylamine). Compound 1-9 can be converted to halide 1-10, where X³ is a halogen (e.g., Cl, Br, or I) under standard halogenation conditions (e.g., in the presence of NBS or NIS or NCS). Alternatively, compound 1-7 can be converted to halide 1-11, where X³ is a halogen (e.g., Cl, Br, or I), under standard halogenation conditions (e.g., in the presence of NBS or NIS or NCS). Ester 1-11 can be hydrolyzed to give the corresponding carboxylic acid by either standard acidic condition (e.g., in the presence of trifluoroacetic acid) or standard basic conditions (e.g., in the presence of lithium hydroxide and water). The acid can be converted to amide 1-10 by standard amide formation conditions (e.g., in the presence of amine 1-8, a coupling reagent, such as hexafluorophosphate azabenzotriazole uronium (HATU), and a base, such as triethylamine). Halide 1-10 can be coupled with compound 1-12 [where A is a cyclic structure (e.g., phenyl, saturated or unsaturated cycloalkyl, saturated or unsaturated heterocycloalkyl, heteroaryl, saturated or unsaturated fused bicyclic ring, or saturated or unsaturated spirocyclic ring, etc.) bearing a reactive functional group Rw (e.g., α,β-unsaturated carbonyl, α,β-unsaturated sulfonyl, halide, nitrile, aldehyde, epoxide, or aziridine, etc.), M² is a boronic acid, boronate ester, potassium trifluoroborate, or an appropriately substituted metal such as Sn(Bu)₃ or Zn] under standard Suzuki conditions (e.g., in the presence of a palladium catalyst, such as XPhos Pd G4 and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compounds of Formula (I).

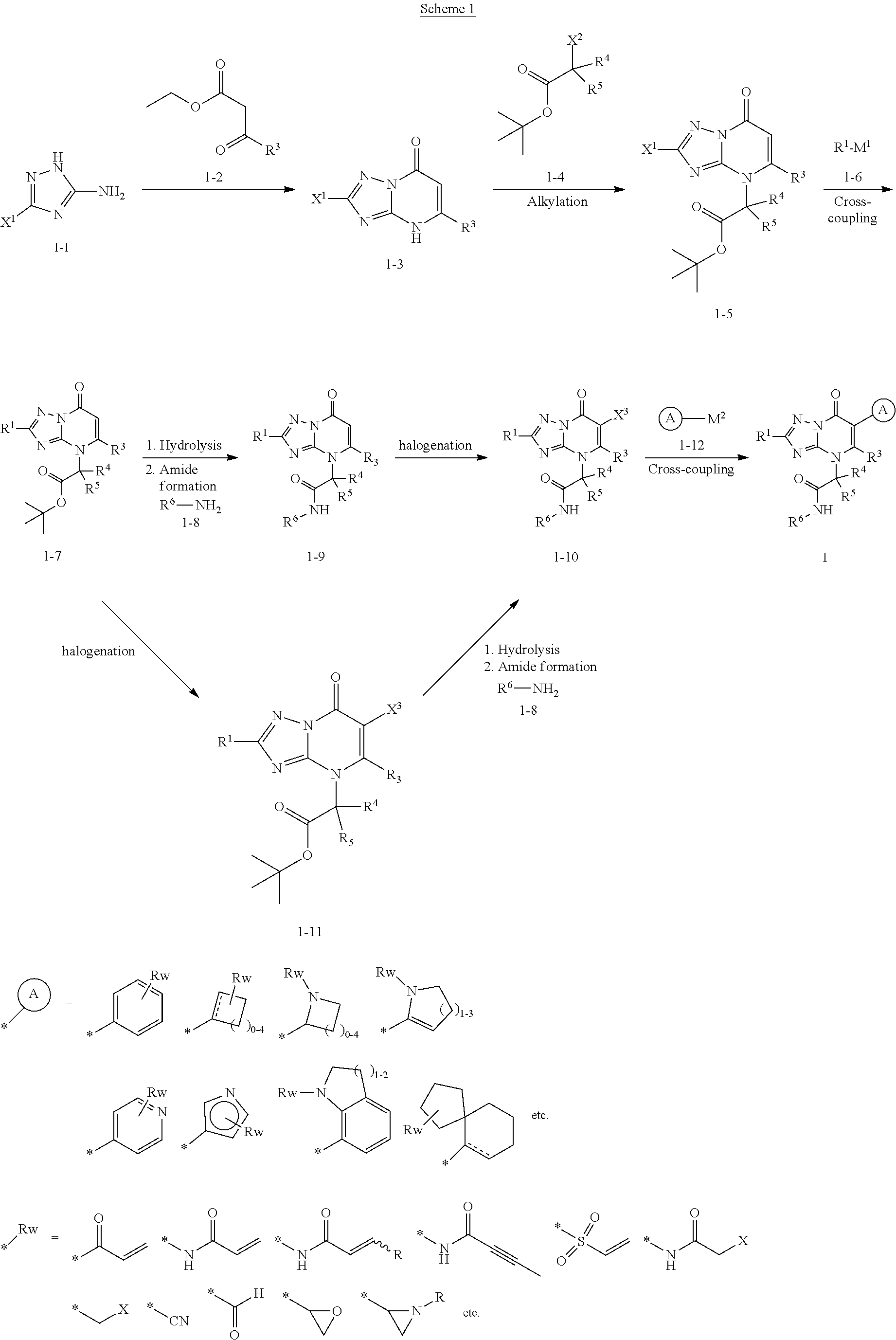

Compounds of Formula (I) can also be prepared as shown in Scheme 2. Suitable starting materials 2-1, which can be synthesized through Scheme 1 (e.g., compound 1-3) where X¹ is a halogen (e.g., Cl, Br, or I), can be alkylated under standard S_{N}2 conditions (e.g., in the presence of diisopropylethylamine and 2-2, where X² is a halogen (e.g., Cl, Br, or I) or pseudohalogen (e.g., OTf or OMs)) to give compound 2-3. Halide 2-3 can be coupled with R¹-M¹ (2-4), where M¹ is a boronic acid, boronate ester, potassium trifluoroborate, or an appropriately substituted metal such as Sn(Bu)₃ or Zn, under standard Suzuki conditions (e.g., in the presence of a palladium catalyst, such as chloro(2-dicyclohexylphosphino-2′,4′,6′-triisopropyl-1,1′-biphenyl)[2-(2′-amino-1,1′-biphenyl)]palladium(II) and a base (e.g., a phosphate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compound 2-5. Compound 2-5 can be converted to halide 2-6, where X³ is a halogen (e.g., Cl, Br, or I) under standard halogenation conditions (e.g., in the presence of NBS or NIS or NCS). Halide 2-6 can be coupled with compound 2-7 [where A is a cyclic structure (e.g., phenyl, saturated or unsaturated cycloalkyl, saturated or unsaturated heterocycloalkyl, heteroaryl, saturated or unsaturated fused bicyclic ring, or saturated or unsaturated spirocyclic ring, etc.) bearing a reactive functional group Rw (e.g., α,β-unsaturated carbonyl, α,β-unsaturated sulfonyl, halide, nitrile, aldehyde, epoxide, or aziridine, etc.), M² is a boronic acid, boronate ester, potassium trifluoroborate, or an appropriately substituted metal such as Sn(Bu)₃ or Zn] under standard Suzuki conditions (e.g., in the presence of a palladium catalyst, such as XPhos Pd G4 and a base (e.g., a phosphate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compounds of Formula (I).

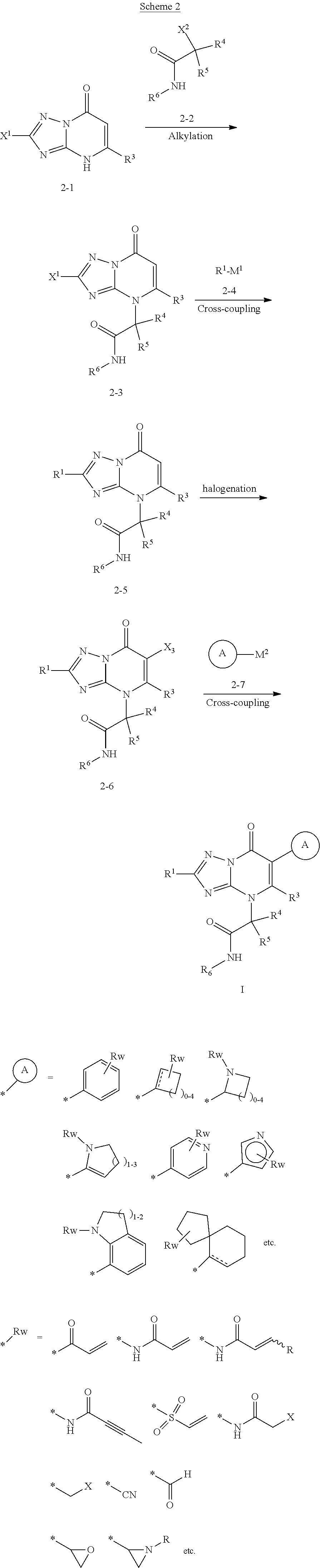

Compounds of Formula (I) can also be prepared as shown in Scheme 3. Condensation of aminotriazole 3-1 with dicarbonyl compound 3-2 at elevated temperatures (e.g., in the presence of acetic acid) can afford bicyclic 3-3. Compound 3-3 can be alkylated under standard S_{N}2 conditions (e.g., in the presence of diisopropylethylamine and halide 3-4, where X² is a halogen (e.g., Cl, Br, or I) or pseudohalogen (e.g., OTf or OMs)) to give compound 3-5. Compound 3-5 can be converted to compound 3-6, where X³ is a halogen (e.g., Cl, Br, or I) under standard halogenation conditions (e.g., in the presence of NBS or NIS or NCS). Compound 3-6 can be coupled with compound 3-7 [where A is a cyclic structure (e.g., phenyl, saturated or unsaturated cycloalkyl, saturated or unsaturated heterocycloalkyl, heteroaryl, saturated or unsaturated fused bicyclic ring, or saturated or unsaturated spirocyclic ring, etc.) bearing a reactive functional group Rw (e.g., α,β-unsaturated carbonyl, α,β-unsaturated sulfonyl, halide, nitrile, alkene, alkyne, aldehyde, epoxide, or aziridine, etc.), M² is a boronic acid, boronate ester, potassium trifluoroborate, or an appropriately substituted metal such as Sn(Bu)₃ or Zn] under standard Suzuki conditions (e.g., in the presence of a palladium catalyst, such as XPhos Pd G4 and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compounds of Formula (I).

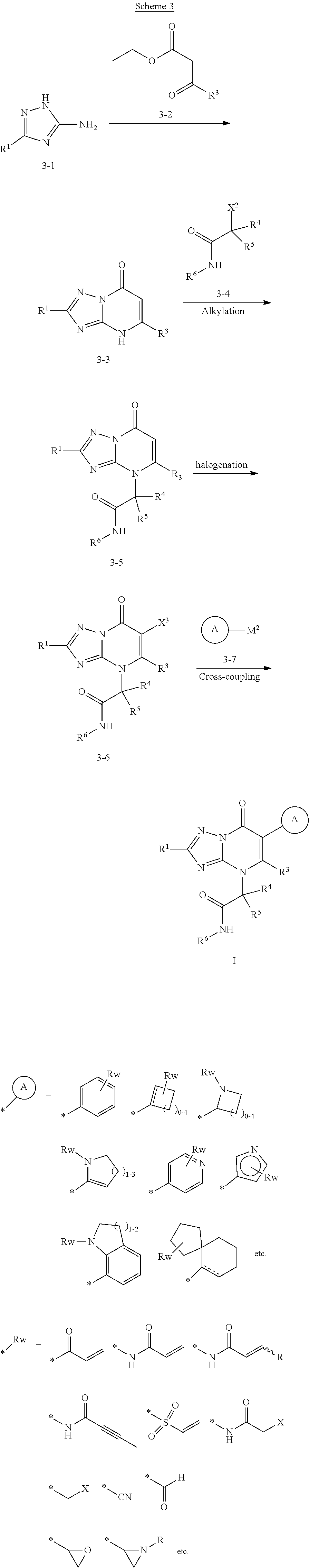

Compounds of Formula (I) can also be prepared as shown in Scheme 4. Suitable starting materials 4-1, which can be synthesized through Scheme 1 (e.g., compound 1-10), Scheme 2 (e.g., compound 2-6), or Scheme 3 (e.g., compound 3-6), where X³ is a halogen (e.g., Cl, Br, or I), can be coupled with compound 4-2 [where D is a cyclic structure bearing an amine that can be part of the ring or a substituent on the ring (e.g., aniline, benzylic amine, cycloalkylamine, cycloalkenylamine, pyridylamine, azetidine, pyrrolidine, piperidine, piperazine, cycloheteroalkylamine, cycloheteroalkenylamine, cycloheteroarylamine, fused bicyclic amine, or spirocyclic amine, etc.), M² is a boronic acid, boronate ester, potassium trifluoroborate, H or an appropriately substituted metal such as Sn(Bu)₃ or Zn] under standard Suzuki conditions (e.g., in the presence of a palladium catalyst, such as XPhos Pd G4 and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)) or standard Buchward-Hartwig conditions (e.g., in the presence of a palladium catalyst, such as [1,1′-bis(diphenylphosphino)ferrocene]dichloropalladium (II)) and a base (e.g., sodium tert-butoxide)) to give compound 4-3. Amine 4-3 can be converted to compounds of Formula (I) under standard amide/sulfonamide formation conditions (e.g., in the presence of a carboxylic chloride or sulfonyl chloride).

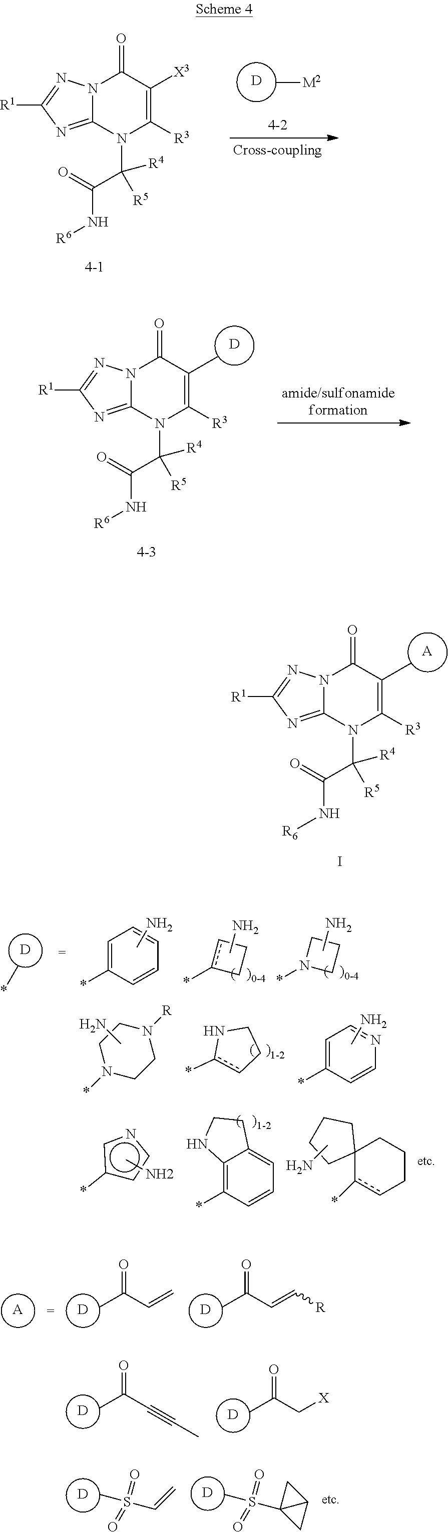

Compounds of Formula (I) can also be prepared, for example, as shown in Scheme 5. Suitable starting materials 5-1, which can be synthesized through Scheme 1 (e.g., compound 1-10), Scheme 2 (e.g., compound 2-6), or Scheme 3 (e.g., compound 3-6), where X³ is a halogen (e.g., Cl, Br, or I), can be coupled with compound 5-2 [where E is a cyclic alkene bearing an amine that can be part of the ring or a substituent on the ring (e.g, cycloalkenylamine or cycloheteroalkenylamine, etc.), M² is a boronic acid, boronate ester, potassium trifluoroborate, or an appropriately substituted metal such as Sn(Bu)₃ or Zn] under standard Suzuki conditions (e.g., in the presence of a palladium catalyst, such as XPhos Pd G4 and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compound 5-3. Alkene moiety on ring E of 5-3 can be saturated to give compound 5-4 under standard hydrogenation condition (e.g., in the presence of hydrogen gas and Pd/C). Amine 5-4 can be converted to compounds of Formula (I) under standard amide/sulfonamide formation conditions (e.g., in the presence of a carboxylic chloride or sulfonyl chloride).

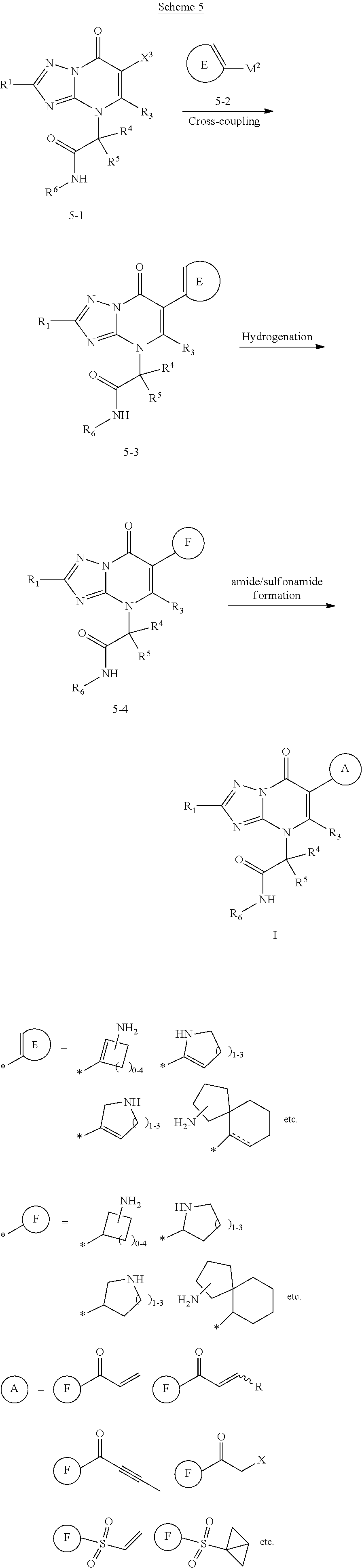

The reactions for preparing compounds of the present disclosure can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of compounds of the present disclosure can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J Chem. Educ., 1997, 74(11), 1297; T. W. Greene and P. G. M. Wuts, Protective Groups in Organic Synthesis, 3^{rd} Ed., Wiley & Sons, Inc., New York (1999); and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC). Compounds can be purified by those skilled in the art by a variety of methods, including high performance liquid chromatography (HPLC) and normal phase silica chromatography.

### Methods of Use

Compounds of the present disclosure can inhibit the activity of WRN and, thus, are useful in treating diseases and disorders associated with activity of PD-1 and the diseases and disorders associated with WRN. In some embodiments, the present disclosure provides a method for inhibiting WRN. In some embodiments, the method includes administering to an individual or a patient a compound of Formula (I), or a pharmaceutically acceptable salt thereof. In some embodiments, the inhibition of WRN is inhibition of WRN helicase activity. In some embodiments, the inhibition of WRN is inhibition of WRN ATPase activity. In some embodiments, the inhibition of WRN is inhibition of WRN helicase activity and WRN ATPase activity.

In some embodiments, the present disclosure provides methods of treating a disease or disorder associated with WRN activity, said method comprising administering to a patient in need thereof a therapeutically effective amount of a compound of Formula (I), or a pharmaceutically acceptable salt thereof. In some embodiments, the WRN activity is WRN helicase activity. In some embodiments, the WRN activity is WRN ATPase activity. In some embodiments, the WRN activity is WRN helicase activity and WRN ATPase activity.

In some embodiments, the present disclosure provides treatment of an individual or a patient in vivo using a compound of Formula (I) or a pharmaceutically acceptable salt thereof such that growth of cancerous tumors is inhibited. A compound of Formula (I) or of any of the formulas as described herein, or a compound as recited in any of the claims and described herein, or a pharmaceutically acceptable salt thereof, can be used to inhibit the growth of cancerous tumors. Alternatively, a compound of Formula (I) or of any of the formulas as described herein, or a compound as recited in any of the claims and described herein, or a pharmaceutically acceptable salt thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a pharmaceutically acceptable salt thereof. In another embodiment, the present disclosure provides a method for inhibiting growth of tumor cells in an individual or a patient. The method includes administering to the individual or patient in need thereof a therapeutically effective amount of a compound of Formula (I) or of any of the formulas as described herein, or of a compound as recited in any of the claims and described herein, or a pharmaceutically acceptable salt thereof.

In some embodiments, provided herein is a method for treating cancer. In some embodiments, the method comprises administering to a patient in need thereof, a therapeutically effective amount of a compound of Formula (I), or a salt thereof. In some embodiments, the cancer is selected from cancers whose growth may be inhibited using compounds of the disclosure. In some embodiments, the cancer is selected from cancers that are classified as microsatellite instability-high (MSI-H) cancers. In some embodiments, the cancer is selected from cancers that are classified as mismatch repair deficient (dMMR) cancers.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T -cell lymphoma, and environmentally induced cancers including those induced by asbestos. In some embodiments, In some embodiments, the patient has not received previous treatments for the cancer. In some embodiments, the patient has not progress from previous treatments for the cancer. In some embodiments, the patient is intolerant to other therapies for the cancer.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, colon cancer, lung cancer (e.g. non-small cell lung cancer and small cell lung cancer), squamous cell head and neck cancer, urothelial cancer (e.g. bladder), and cancers with high microsatellite instability and/or mismatch repair deficient (MSI-H/dMMR)). In some embodiments, the cancer being treated includes refractory or recurrent malignancies whose growth may be inhibited using the compounds of the present disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma, or multiple myeloma.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, hematological cancers, sarcomas, lung cancers, gastrointestinal cancers, genitourinary tract cancers, liver cancers, bone cancers, nervous system cancers, gynecological cancers, and skin cancers.

Examples of hematological cancers that are treatable using the compounds of the present disclosure include lymphomas and leukemias, such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Examples of sarcomas that are treatable using the compounds of the present disclosure include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Examples of lung cancers that are treatable using the compounds of the present disclosure include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Examples of gastrointestinal cancers that are treatable using the compounds of the present disclosure include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Examples of genitourinary tract cancers that are treatable using the compounds of the present disclosure include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Examples of liver cancers that are treatable using the compounds of the present disclosure include hepatoma (hepatocellular carcinoma), cholangiocarcinoma, hepatoblastoma, angiosarcoma, hepatocellular adenoma, and hemangioma.

Examples of bone cancers that are treatable using the compounds of the present disclosure include osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Examples of nervous system cancers that are treatable using the compounds of the present disclosure include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Examples of gynecological cancers that are treatable using the compounds of the present disclosure include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Examples of skin cancers that are treatable using the compounds of the present disclosure include melanoma, basal cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

The terms “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the present disclosure are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Formulation, Compositions, Dosage Forms, and Administration

When employed as pharmaceuticals, the compounds of the present disclosure can be administered in the form of pharmaceutical compositions. Thus, the present disclosure provides a composition comprising a compound of Formula (I) or any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The present disclosure also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the present disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the present disclosure may be milled using known milling procedures such as wet milling to obtain a particle size appropriate for tablet formation and for other formulation types. Finely divided (nanoparticulate) preparations of the compounds of the present disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the present disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g. Methocel KOOLV™). In some embodiments, the polyethylene oxide is polyethylene oxide WSR 1105 (e.g., Polyox WSR 1105™)

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the present disclosure in a pharmaceutical composition can vary depending upon a number of factors including dosage, chemical characteristics (e.g., hydrophobicity), and the route of administration. For example, the compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills, and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the present disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the present disclosure in a pharmaceutical composition can vary depending upon a number of factors including dosage, chemical characteristics (e.g., hydrophobicity), and the route of administration. For example, the compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### Patient Screening

In some embodiments, therapeutic applications of the WRN inhibition compounds of the present disclosure can be accompanied by one or more screening tests, including patient screening tests according to clinical practice guidance. In some embodiments, one or more screening test can be administered to determine if the cancer is a microsatellite instability-high (MSI-H) cancer. Polymerase chain reaction (PCR) and immunohistochemistry (IHC) assays can be used to classify if a cancer is MSI.

In some embodiments, a polymerase chain reaction (PCR) assay is used to determine if the cancer is a microsatellite instability-high (MSI-H) cancer. The PCR based assays can follow the NCI-Reference Panel and/or the Promega MSI Analysis System. The NCI-Reference Panel (also referred as Bethesda Panel, Bethesda 1998. Ref 3) includes five loci, three dinucleotide sites (D2S123, D5S346 and D17S250) and two mononucleotide sites (BAT-25 and BAT-26). The Promega MSI Analysis System (such as OncoMate™ kit) includes five mononucleotide repeat markers: BAT-25, BAT-26, NR-21, NR-24 and MONO-27. A cancer can be classified as MSI if a downward shift in size is detected in at least two of the five genes, compared to normal tissue samples.

In some embodiments, an immunohistochemistry (IHC) assay is used to determine if the cancer is a microsatellite instability-high (MSI-H) cancer. The IHC assay can be used to detect loss of protein expression of mismatch repairs genes, such as MLH1, MSH2, MSH6 and PMS2. These genes are also associated with hereditary nonpolyposis colorectal cancer (HNPCC), Lynch syndrome, and the constitutional MMR deficiency (CMMRD) syndrome. In some embodiments, a loss of expression with at least one of the four proteins, compared to normal tissue samples, can classify a cancer as MSI.

In some embodiments, one or more screening test can be administered to determine if the cancer is a mismatch repair deficient (dMMR) cancer.

### Combination Therapies

In some embodiments, the WRN inhibitor compounds of the present disclosure are administered as a single agent. In some embodiments, the WRN inhibitor compound is administered in combination with an additional active agent or treatment. In some embodiments, the WRN inhibitor compound is administered in combination with an immunotherapy, a targeted therapy, a chemotherapy, or radiation therapy. In some embodiments, the active agents of the combination therapy have different and complementary mechanisms of action. In some embodiments, the active agents of the combination therapy have a synergistic and/or more durable therapeutic effect on a target disease, disorder and condition. In some embodiments, the combination of active agents allows for a dose adjustment of one or more of the active agents, which can reduce adverse effects associated with one or more of the active agents.

In some embodiments, the active agents can be co-administered in a single composition. In some embodiments, the active agents can be co-administered separately. In some embodiments, the active agents can be co-administered separately and simultaneously. In some embodiments, the active agents can be administered separately and sequentially.

Combination with Immune-Checkpoint Therapies

In some embodiments, WRN inhibitor compounds of the present disclosure are used in combination with one or more immune checkpoint inhibitors for the treatment of diseases, such as cancer or infections. Examples of immune checkpoint inhibitors include inhibitors against immune checkpoint molecules such as CBL-B, CD20, CD28, CD40, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TIGIT, TLR (TLR7/8), CD112R, VISTA, PD-1, PD-L1, and PD-L2. In some embodiments, the immune checkpoint inhibitor is a stimulatory checkpoint molecule selected from CD27, CD28, CD40, ICOS, OX40, GITR, and CD137. In some embodiments, the immune checkpoint inhibitor is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the WRN inhibitor compounds are used in combination with one or more agents selected from KIR inhibitors, TIGIT inhibitors, LAIR1 inhibitors, CD160 inhibitors, 2B4 inhibitors, and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MED19447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MED10562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

Combination with Other Therapies

In some embodiments, the WRN inhibitor compounds of the present disclosure can be used in combination with other methods of treating cancers. Examples of other methods of treating cancer include chemotherapy, irradiation therapy, tumor-targeted therapy, adjuvant therapy, immunotherapy, surgery, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors. compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFPR, PI3K (alpha, beta, gamma, delta, and multiple or selective), CSF1R, KIT, FLK-II, KDR/FLK-1, FLK-4, fit-1, FGFRI, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lek, Fgr, Btk, Fak, SYK, FRK, JAK, ABL, ALK and B-Raf. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, STING agonists, RIG-I agonists, oncolytic virotherapy and immunomodulating small molecules, including thalidomide or JAK1/2 inhibitor, PI3Kδ inhibitor, and the like.

In some embodiments, the WRN inhibitor compounds are administered in combination with one or more anti-cancer drugs, such as a chemotherapeutic agent. Examples of chemotherapeutic agents include: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

### Labeled Compounds and Assay Methods

The compounds of the present disclosure can further be useful in investigations of biological processes in normal and abnormal tissues. Thus, another aspect of the present disclosure relates to labeled compounds of the present disclosure (radio-labeled, fluorescent-labeled, etc.) that would be useful not only in imaging techniques but also in assays, both in vitro and in vivo, for localizing and quantitating WRN protein in tissue samples, including human, and for identifying WRN ligands by inhibition binding of a labeled compound. Accordingly, the present disclosure includes WRN binding assays that contain such labeled compounds.

The present disclosure further includes isotopically-labeled compounds of the disclosure. An “isotopically” or “radio-labeled” compound is a compound of the present disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as -CD3 being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro WRN protein labeling and competition assays, compounds that incorporate ³H, ¹⁴C, ⁸²Br, ¹²⁵I, ¹³¹I ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the present disclosure can be used in a screening assay to identify and/or evaluate compounds. For example, a newly synthesized or identified compound (i.e., test compound) which is labeled can be evaluated for its ability to bind a WRN protein by monitoring its concentration variation when contacting with the WRN protein, through tracking of the labeling. For example, a test compound (labeled) can be evaluated for its ability to reduce binding of another compound which is known to bind to a WRN protein (i.e., standard compound). Accordingly, the ability of a test compound to compete with the standard compound for binding to the WRN protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### EXAMPLES

Certain embodiments of the present disclosure will be described in greater detail by way of specific examples. The following examples are offered for illustrative purposes and are not intended to limit the present disclosure in any manner. Those of skill in the art will readily recognize a variety of non-critical parameters which can be changed or modified to yield essentially the same results. The compounds of the Examples have been found to inhibit the activity of WRN according to at least one assay described herein.

Experimental procedures for compounds of the present disclosure are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al., “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 min with flow rate 2.0 mL/min.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/min, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J Comb. Chem., 6, 874-883 (2004)).

The following abbreviations may be used herein: aq. (aqueous); br (broad); d (doublet); dd (doublet of doublets); DCM (dichloromethane); DMF (N, N-dimethylformamide); Et (ethyl); EtOAc (ethyl acetate); g (gram(s)); h (hrs)); HPLC (high performance liquid chromatography); Hz (hertz); J (coupling constant); LCMS (liquid chromatography—mass spectrometry); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); nM (nanomolar); NMR (nuclear magnetic resonance spectroscopy); Ph (phenyl); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); μg (microgram(s)); μL (microliter(s)); μM (micromolar); wt % (weight percent).

### Example 1. N-(2-(2-(3,6-Dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide

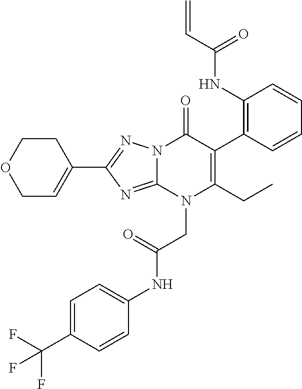 [SMILES: C=CC(=O)Nc1ccccc1-c1c(CC)n(CC(=O)Nc2ccc(C(F)(F)F)cc2)c2nc(C3=CCOCC3)nn2c1=O]

### Step 1. 2-Bromo-5-ethyl-[1,2,4]triazolo[1, 5-a]pyrimidin-7(4H)-one

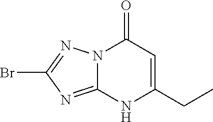

A mixture of 3-bromo-1H-1,2,4-triazol-5-amine (500 mg, 3.07 mmol) and ethyl propionylacetate (0.918 mL, 6.44 mmol) in acetic acid (3.0 mL) was stirred at 80° C. overnight (24 hrs). After cooling, the solids were filtered, washed with water (3.0 mL), and dried to give 300 mg of white solids. The filtrate was concentrated. DCM (10 mL) was added to the filtrate and stirred for 30 min. The solids were filtered, washed with DCM (10 mL) and dried to give 203 mg (67% total) of additional product. LCMS calculated for C₇H₈BrN₄O (M+H)⁺: m/z=243.0, 245.0; found=243.0, 245.0.

### Step 2. tert-Butyl 2-(2-bromo-5-ethyl-7-oxo-[1,2,4]triazolo[1,5-a]pyrimidin-4(7H)-yl)acetate

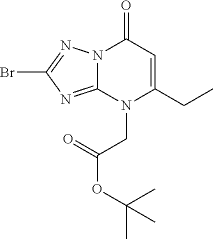 [SMILES: CCc1cc(=O)n2nc(Br)nc2n1CC(=O)OC(C)(C)C]

To a mixture of 2-bromo-5-ethyl-[1,2,4]triazolo[1,5-a]pyrimidin-7(4H)-one (300 mg, 1.23 mmol) and tert-butyl 2-bromoacetate (0.200 mL, 1.36 mmol) stirring in 1,4-dioxane (6 mL) was added N,N-diisopropylethylamine (0.65 mL, 3.70 mmol). The resulting mixture was stirred at room temperature overnight (19 hrs). LCMS showed the reaction was not complete. The mixture was heated to 50° C. (a clear solution formed), and stirred overnight. Cooled to room temperature, then in an ice bath. Water (10 mL) was added, stirred for 1 h, filtered and dried in vacuo to give the crude product as a white solid (270 mg, 61%). LC-MS calculated for C₁₃H₁₈BrN₄O₃ (M+H)⁺: m/z=357.0, 359.0; found 357.0, 359.0. ¹H NMR (400 MHz, DMSO-d₆) δ 6.04 (s, 1H), 5.00 (s, 2H), 2.69 (q, J=7.4 Hz, 2H), 1.43 (s, 9H), 1.19 (t, J=7.4 Hz, 3H).

### Step 3. tert-Butyl 2-(2-(3,6-dihydro-2H-pyran-4-yl)-5-ethyl-7-oxo-[1,2,4]triazolo[1,5-a]pyrimidin-4(7H)-yl)acetate

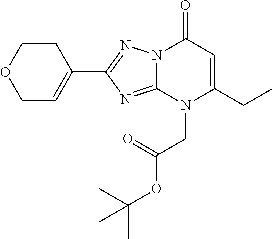 [SMILES: CCc1cc(=O)n2nc(C3=CCOCC3)nc2n1CC(=O)OC(C)(C)C]

To a vial containing tert-butyl 2-(2-bromo-5-ethyl-7-oxo-[1,2,4]triazolo[1,5-a]pyrimidin-4(7H)-yl)acetate (250 mg, 0.700 mmol) was added 2-(3,6-dihydro-2H-pyran-4-yl)-4,4,5,5-tetramethyl-1,3,2-dioxaborolane (221 mg, 1.05 mmol), chloro(2-dicyclohexylphosphino-2′,4′,6′-triisopropyl-1,1′-biphenyl)[2-(2′-amino-1,1′-biphenyl)]palladium(II) (55.1 mg, 0.070 mmol) and potassium phosphate tribasic (371 mg, 1.75 mmol). 1,4-Dioxane (4.2 mL) and water (0.6 mL) were added, degassed/purged with N2, and heated to 80° C. After 1 h, the reaction was complete. The resulting mixture was cooled, then filtered through a pad of celite, washing with ethyl acetate (15 mL). The filtrate was concentrated then purified using flash column chromatography (40 g SiO₂, 10-60% EtOAc/Hex) to give the desired product (246 mg, 98%) as a white amorphous solid. LC-MS calculated for C₁₈H₂₅N₄O₄ (M+H)⁺: m/z=361.2; found 361.2.

### Step 4. 2-(2-(3,6-Dihydro-2H-pyran-4-yl)-5-ethyl-7-oxo-[1,2,4]triazolo[1,5-a]pyrimidin-4(7H)-yl)acetic acid

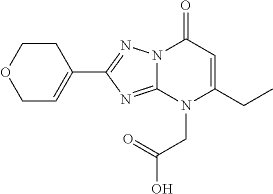 [SMILES: CCc1cc(=O)n2nc(C3=CCOCC3)nc2n1CC(=O)O]

To a solution of tert-butyl 2-(2-(3,6-dihydro-2H-pyran-4-yl)-5-ethyl-7-oxo-[1,2,4]triazolo[1,5-a]pyrimidin-4(7H)-yl)acetate (340 mg, 0.943 mmol) stirring in THF (6 mL) and MeOH (4 mL) was added 1M NaOH in water (1.89 mL, 1.89 mmol). The mixture was stirred overnight at room temperature. Additional 1M NaOH (0.9 mL) was added and stirred for 4 h. Most of the organic solvent was evaporated. Water (5 mL) was added and the mixture was made slightly acidic by the addition of TN HCl (aq.) (2 mL). The aqueous mixture was extracted with DCM (10 mL×3). The combined extracts were dried (MgSO₄), filtered and concentrated to give the crude product (264 mg, 92%). LC-MS calculated for C₁₄H₁₇N₄O₄ (M+H)⁺: m/z=305.1; found 305.1.

### Step 5. 2-(2-(3,6-Dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

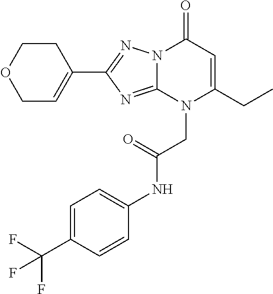 [SMILES: CCc1cc(=O)n2nc(C3=CCOCC3)nc2n1CC(=O)Nc1ccc(C(F)(F)F)cc1]

To a mixture of 2-(2-(3,6-dihydro-2H-pyran-4-yl)-5-ethyl-7-oxo-[1,2,4]triazolo[1,5-a]pyrimidin-4(7H)-yl)acetic acid (264 mg, 0.868 mmol), 4-(trifluoromethyl)aniline (0.163 mL, 1.30 mmol), and triethylamine (0.181 mL, 1.30 mmol) in DMF (8 mL) was added hexafluorophosphate azabenzotriazole uronium (396 mg, 1.041 mmol). Stirred overnight at room temperature. Water (5 mL) was added, causing a solid to precipitate. Added excess water (10 mL), stirred for 30 min then filtered and dried the solids in vacuo to give 329 mg (85%). LC-MS calculated for C₂₁H₂₁F₃N₅O₃ (M+H)⁺: m/z=448.1; found 448.1.

### Step 6. 2-(6-Bromo-2-(3,6-dihydro-2H-pyran-4-yl)-5-ethyl-7-oxo-[1, 2,4]triazolo[1,5-a]pyrimidin-4(7H)-yl)-N-(4-(trifluoromethyl)phenyl)acetamide

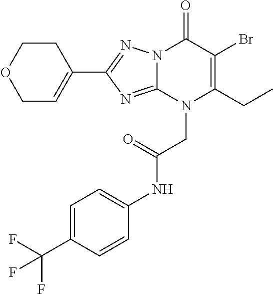 [SMILES: CCc1c(Br)c(=O)n2nc(C3=CCOCC3)nc2n1CC(=O)Nc1ccc(C(F)(F)F)cc1]

To a mixture of 2-(2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (329 mg, 0.735 mmol) in acetonitrile (8 mL) was added N-bromosuccinimide (137 mg, 0.772 mmol). Stirred at room temperature for 4 h. Water (10 mL) was added then extracted with DCM (10 mL×3). The combined extracts were dried (MgSO₄), filtered and concentrated. The crude material was purified using flash column chromatography (40 g SiO₂, 5-100% EtOAc/Hex) to give 205 mg (53%) of a white solid. LC-MS calculated for C₂₁H₂₀BrF₃N₅O₃ (M+H)⁺: m/z=526.1, 528.1; found 526.1, 528.1.

### Step 7. 2-(6-(2-Aminophenyl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

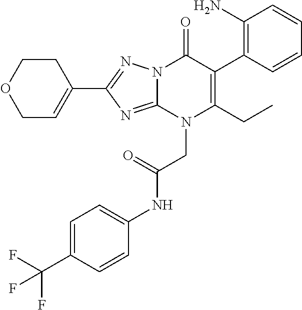 [SMILES: CCc1c(-c2ccccc2N)c(=O)n2nc(C3=CCOCC3)nc2n1CC(=O)Nc1ccc(C(F)(F)F)cc1]

2-(6-Bromo-2-(3,6-dihydro-2H-pyran-4-yl)-5-ethyl-7-oxo-[1,2,4]triazolo[,5-a]pyrimidin-4(7H)-yl)-N-(4-(trifluoromethyl)phenyl)acetamide (57 mg, 0.108 mmol), (2-aminophenyl)boronic acid (22.3 mg, 0.162 mmol), XPhos Pd G4 (9.32 mg, 10.8 μmol) and potassium phosphate tribasic (46.0 mg, 0.217 mmol) were weighed into a vial. 1,4-Dioxane (1.0 mL) and water (160 μL) were added, purged with N₂, and heated to 90° C. for 4 h. After cooling, the mixture was filtered through a pad of celite, washing with EtOAc (10 mL), and concentrated. The crude was purified by flash column chromatography (40 g SiO₂, 5-100% EtOAc/Hex, then switched to 0-10% MeOH/DCM to elute the product). Isolated 26 mg (45%) of the desired product and some mixed fractions containing the product. The mixed fractions were purified using preparative LCMS (pH 2) to give additional product. LC-MS calculated for C₂₇H₂₆F₃N₆O₃ (M+H)⁺: m/z=539.2; found 539.2. ¹H NMR (400 MHz, DMSO-d₆) δ 10.82 (s, 1H), 7.81 (d, J=8.6 Hz, 2H), 7.72 (d, J=8.7 Hz, 2H), 7.21-7.12 (m, 1H), 6.99 (d, J=7.4 Hz, 1H), 6.86-6.79 (m, 2H), 6.74 (t, J=7.4 Hz, 1H), 5.30-5.18 (m, 2H), 4.26 (q, J=2.8 Hz, 3H), 3.85-3.77 (m, 3H), 2.66-2.57 (m, 1H), 2.54 (s, 2H), 2.43-2.38 (m, 1H), 1.01 (t, J=7.5 Hz, 3H).

### Step 8. N-(2-(2-(3,6-Dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide

To a mixture of 2-(6-(2-aminophenyl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (10 mg, 0.019 mmol) in DCM (0.20 mL) stirring at about 0° C. was added diisopropylethylamine (4.86 pl, 0.028 mmol), then acryloyl chloride (1.81 pl, 0.022 mmol). The ice bath was removed and the mixture stirred for 20 min. Solvents were evaporated and the residue purified using preparatory LCMS (pH 2) to give 4.2 mg (37%) of the desired product. LC-MS calculated for C₃₀H₂₈F₃N₆O₄ (M+H)⁺: m/z=593.2; found 593.3. ¹H NMR (400 MHz, DMSO-d₆) δ 10.85 (s, 1H), 9.08 (s, 1H), 8.12 (d, J=8.2 Hz, 1H), 7.80 (d, J=8.6 Hz, 2H), 7.73 (d, J=8.7 Hz, 2H), 7.47-7.38 (m, 1H), 7.25 (d, J=4.1 Hz, 2H), 6.84 (s, 1H), 6.50 (dd, J=17.0, 10.2 Hz, 1H), 6.20 (dd, J=17.0, 2.1 Hz, 1H), 5.69 (dd, J=10.2, 2.1 Hz, 1H), 5.29 (d, J=17.4 Hz, 1H), 5.21 (d, J=17.3 Hz, 1H), 4.27 (d, J=3.0 Hz, 2H), 3.87-3.77 (m, 2H), 2.55 (s, 2H), 2.35-2.30 (m, 1H), 0.94 (t, J=7.4 Hz, 3H).

### Examples 2 & 3

Example compounds 2 and 3 in Table 1 were prepared according to the procedures described in Example 1, using appropriately substituted starting materials.

TABLE 1

| Ex. | | | LCMS | |
|---|---|---|---|---|
| No. | Name | Structure | [M + H]⁺ | ¹H NMR |
| 2 | 2-(2-(3,6-Dihydro- 2H-pyran-4-yl)-5- ethyl-7-oxo-6-(2- (vinylsulfonamido) phenyl)- [1,2,4]triazolo[1,5- a]pyrimidin-4(7H)- yl)-N-(4- (trifluoromethyl) phenyl)acetamide | 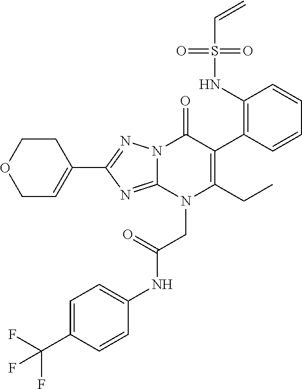 [SMILES: C=CS(=O)(=O)Nc1ccccc1-c1c(CC)n(CC(=O)Nc2ccc(C(F)(F)F)cc2)c2nc(C3=CCOCC3)nn2c1=O] | 629.1 | |
| 3 | Ethyl (E)-4-((2-(2- (3,6-dihydro-2H- pyran-4-yl)-5- ethyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)amino)- 4-oxobut-2-enoate | 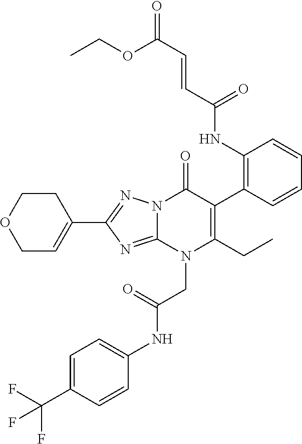 [SMILES: CCOC(=O)/C=C/C(=O)Nc1ccccc1-c1c(CC)n(CC(=O)Nc2ccc(C(F)(F)F)cc2)c2nc(C3=CCOCC3)nn2c1=O] | 665.2 | ¹H NMR (400 MHz, DMSO- d₆) δ 10.87 (s, 1H), 9.58 (s, 1H), 8.08 (d, J = 8.2 Hz, 1H), 7.80 (d, J = 8.5 Hz, 2H), 7.68 (d, J = 8.5 Hz, 2H), 7.49-7.43 (m, J = 8.6, 4.5 Hz, 1H), 7.34 (d, J = 15.4 Hz, 1H), 7.30 (d, J = 4.4 Hz, 2H), 6.85 (s, 1H), 6.68 (d, J = 15.5 Hz, 1H), 5.33 (d, J = 17.3 Hz, 1H), 5.19 (d, J = 17.3 Hz, 1H), 4.27 (d, J = 2.8 Hz, 2H), 4.24-4.14 (m, 2H), 2.55 (s, 2H), 2.31 (dd, J = 14.5, 7.3 Hz, 1H), 1.22 (t, J = 7.1 Hz, 3H), 0.93 (t, J = 7.5 Hz, 3H). |

### Examples 4 & 5. (Z)-2-Cyano-N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-4-methylpent-2-enamide; and (E)-2-Cyano-N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-4-methylpent-2-enamide

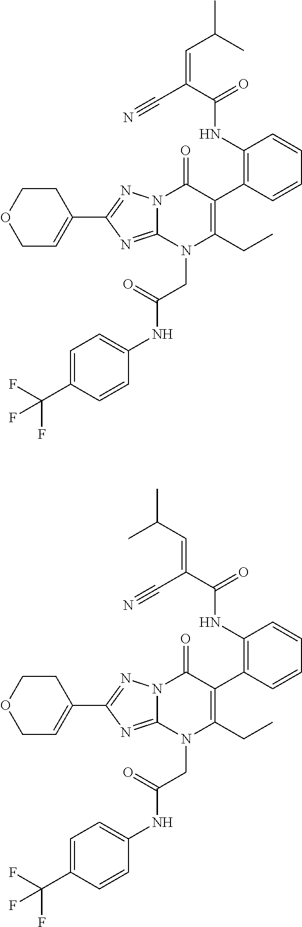 [SMILES: CCc1c(-c2ccccc2NC(=O)/C(C#N)=C\C(C)C)c(=O)n2nc(C3=CCOCC3)nc2n1CC(=O)Nc1ccc(C(F)(F)F)cc1 ; CCc1c(-c2ccccc2NC(=O)/C(C#N)=C/C(C)C)c(=O)n2nc(C3=CCOCC3)nc2n1CC(=O)Nc1ccc(C(F)(F)F)cc1]

To a mixture of 2-(6-(2-aminophenyl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (10 mg, 0.019 mmol) (Example 1, step 7), 2-cyano-4-methylpent-2-enoic acid (3.10 mg, 0.022 mmol), and triethylamine (10.4 pl, 0.074 mmol) in acetonitrile (0.20 mL) was added n-propylphosphonic acid anhydride, cyclic trimer (0.033 mL, 0.056 mmol). Stirred at room temperature overnight. The reaction was not complete so additional n-propylphosphonic acid anhydride, cyclic trimer (0.033 mL, 0.056 mmol) was added and heated the mixture to 40° C. After 4 h, the mixture was diluted with acetonitrile and purified using preparatory LCMS (pH 2) to give 2 peaks with the desired mass. Peak 1 (Example 4): Isolated 2.7 mg (22%); LC-MS calculated for C₃₄H₃₃F₃N₇O₄ (M+H)⁺: m/z=660.2; found 660.2. Peak 2 (Example 5): Isolated 2.9 mg (24%); LC-MS calculated for C₃₄H₃₃F₃N₇O₄ (M+H)⁺: m/z=660.2; found 660.2. ¹H NMR (600 MHz, DMSO-d₆) δ 10.87 (s, 1H), 9.63 (s, 1H), 7.81 (d, J=8.5 Hz, 2H), 7.70 (dd, J=8.3, 6.4 Hz, 3H), 7.47 (ddd, J=8.3, 5.7, 3.3 Hz, 1H), 7.39-7.32 (m, 2H), 7.17 (d, J=10.0 Hz, 1H), 6.85-6.81 (m, 1H), 5.33 (d, J=17.3 Hz, 1H), 5.13 (d, J=17.3 Hz, 1H), 4.26 (q, J=2.9 Hz, 2H), 3.81 (t, J=5.5 Hz, 2H), 2.80-2.70 (m, 1H), 2.53 (m, 3H), 2.43-2.35 (m, 1H), 1.08-0.90 (m, 9H).

### Example 6. N-(2-(2-(3,6-Dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-N-methylacrylamide

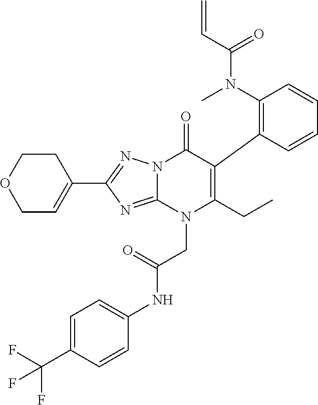 [SMILES: C=CC(=O)N(C)c1ccccc1-c1c(CC)n(CC(=O)Nc2ccc(C(F)(F)F)cc2)c2nc(C3=CCOCC3)nn2c1=O]

### Step 1. 2-(2-(3,6-dihydro-2H-pyran-4-yl)-5-ethyl-6-(2-(methylamino)phenyl)-7-oxo-[1,2,4]triazolo[1,5-a]pyrimidin-4(7H)yl)-N-(4-(trifluoromethyl)phenyl)acetamide

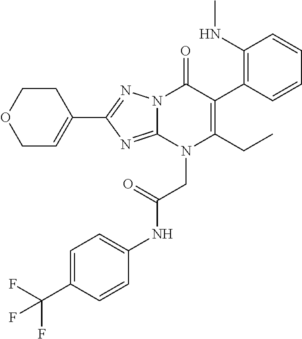 [SMILES: CCc1c(-c2ccccc2NC)c(=O)n2nc(C3=CCOCC3)nc2n1CC(=O)Nc1ccc(C(F)(F)F)cc1]

2-(6-Bromo-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (30 mg, 0.057 mmol)(Example 1, Step 6), N-methyl-2-(4,4,5,5-tetramethyl-1,3,2-dioxaborolan-2-yl)aniline (19.9 mg, 0.086 mmol), XPhos Pd G4 (4.90 mg, 5.70 μmol) and potassium phosphate tribasic (24.2 mg, 0.114 mmol) were weighed into a vial. 1,4-Dioxane (0.60 mL) and water (80 μL) were added, purged with N₂, and heated to 90° C. for 22 h. After cooling, the mixture was passed through a Si-Thiol cartridge, eluting with methanol (2 mL) and acetonitrile (2 mL). The eluent was purified using prep-LCMS (pH 2) to give 8.3 mg (26%) of the desired product. LC-MS calculated for C₂₈H₂₈F₃N₆O₃ (M+H)⁺: m/z=553.2; found 553.2.

### Step 2. N-(2-(2-(3,6-Dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-N-methylacrylamide

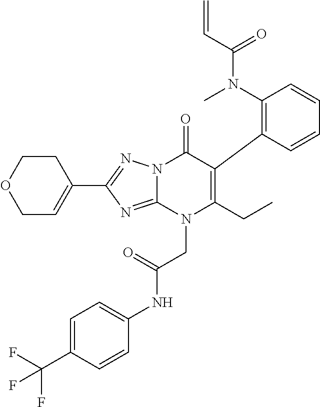 [SMILES: C=CC(=O)N(C)c1ccccc1-c1c(CC)n(CC(=O)Nc2ccc(C(F)(F)F)cc2)c2nc(C3=CCOCC3)nn2c1=O]

A solution of 2-(2-(3,6-dihydro-2H-pyran-4-yl)-5-ethyl-6-(2-(methylamino)phenyl)-7-oxo-[1,2,4]triazolo[1,5-a]pyrimidin-4(7H)-yl)-N-(4-(trifluoromethyl)phenyl)acetamide (7 mg, 0.013 mmol) in DCM (0.30 mL) was cooled in an ice bath to about 0° C. before adding diethylpropylamine (6.64 μl, 0.038 mmol), then acryloyl chloride (1.44 μl, 0.018 mmol). The ice bath was removed and the mixture stirred for 2 h at room temperature. The solvent was evaporated and the residue purified using preparatory LCMS (pH 2) to give 4 mg (52%) of the desired product. LC-MS calculated for C₃₁H₃₀F₃N₆O₄ (M+H)⁺: m/z=607.2; found 607.3. ¹H NMR (400 MHz, DMSO-d₆) δ 10.91 (s, 1H), 7.79 (d, J=8.5 Hz, 2H), 7.72 (d, J=8.6 Hz, 2H), 7.60-7.39 (m, 3H), 7.30-7.24 (m, 1H), 6.83 (s, 1H), 6.23 (dd, J=17.0, 10.3 Hz, 1H), 6.10-5.98 (m, 1H), 5.49 (dd, J=10.3, 2.5 Hz, 1H), 5.41 (d, J=17.4 Hz, 1H), 5.11 (dd, J=17.3, 12.0 Hz, 1H), 4.25 (q, J=2.9 Hz, 2H), 3.79 (t, J=5.2 Hz, 3H), 2.97 (s, 3H), 2.68 (q, J=7.7 Hz, 1H), 2.31 (dd, J=14.6, 7.5 Hz, 1H), 1.21 (q, J=9.1 Hz, 3H), 1.12 (t, J=7.4 Hz, 1H).

### Examples 7-13

Example compounds 7-13 in Table 2 were prepared according to the procedures described in Example 6, using appropriately substituted starting materials in Step 1.

TABLE 2

| Ex. | | | LCMS | |
|---|---|---|---|---|
| No. | Name | Structure | [M + H]⁺ | ¹H NMR |
| 7 | N-(2-(2-(3,6- Dihydro-2H-pyran- 4-yl)-5-ethyl-7- oxo-4-(2-oxo-2- ((4- (trifluoromethyl) phenyl)amino)ethyl)- 4,7-dihydro- [1,2,4]triazolo[1,5- a]pyrimidin-6-yl)- 6- methylphenyl)acryl amide | 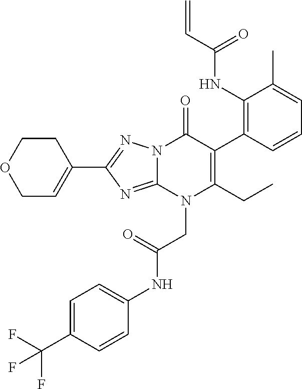 [SMILES: C=CC(=O)Nc1c(C)cccc1-c1c(CC)n(CC(=O)Nc2ccc(C(F)(F)F)cc2)c2nc(C3=CCOCC3)nn2c1=O] | 607.3 | ¹H NMR (400 MHz, DMSO- d₆) δ 10.82 (s, 1H), 9.36 (s, 1H), 7.85-7.68 (m, 4H), 7.37- 7.27 (m, 2H), 7.15 (d, J = 7.2 Hz, 1H), 6.82 (s, 1H), 6.39 (dd, J = 17.1, 10.2 Hz, 1H), 6.06 (dd, J = 17.1, 2.2 Hz, 1H), 5.59 (dd, J = 10.2, 2.2 Hz, 1H), 5.31 (d, J = 17.4 Hz, 1H), 5.09 (d, J = 17.3 Hz, 1H), 4.26 (d, J = 2.9 Hz, 2H), 3.81 (t, J = 5.4 Hz, 2H), 2.47- 2.32 (m, 2H), 2.19 (s, 3H), 0.96 (t, J = 7.5 Hz, 3H). |
| 8 | N-(2-(2-(3,6- Dihydro-2H-pyran- 4-yl)-5-ethyl-7- oxo-4-(2-oxo-2- ((4- (trifluoromethyl) phenyl)amino)ethyl)- 4,7-dihydro- [1,2,4]triazolo[1,5- a]pyrimidin-6-yl)- 5- methylphenyl)acryl amide | 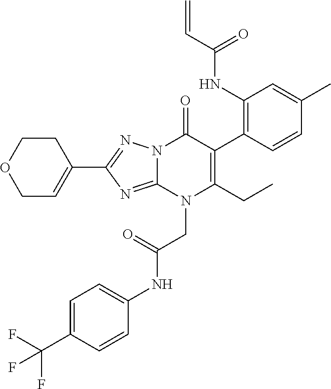 [SMILES: C=CC(=O)Nc1cc(C)ccc1-c1c(CC)n(CC(=O)Nc2ccc(C(F)(F)F)cc2)c2nc(C3=CCOCC3)nn2c1=O] | 607.3 | ¹H NMR (400 MHz, DMSO- d₆) δ 10.86 (d, J = 16.4 Hz, 1H), 9.01 (d, J = 3.7 Hz, 1H), 7.95 (s, 1H), 7.80 (d, J = 8.8 Hz, 2H), 7.72 (d, J = 8.7 Hz, 2H), 7.12 (d, J = 8.0 Hz, 1H), 7.10-7.03 (m, 1H), 6.84 (s, 1H), 6.55-6.42 (m, 1H), 6.19 (dd, J = 17.0, 2.1 Hz, 1H), 5.68 (dd, J = 10.1, 2.3 Hz, 1H), 5.28 (d, J = 17.4 Hz, 1H), 5.20 (d, J = 17.2 Hz, 1H), 4.26 (d, J = 3.0 Hz, 2H), 3.87- 3.78 (m, 2H), 2.54 (s, 2H), 2.37 (s, 3H), 0.94 (q, J = 7.3 Hz, 3H). |
| 9 | N-(2-(2-(3,6- Dihydro-2H-pyran- 4-yl)-5-ethyl-7- oxo-4-(2-oxo-2- ((4- (trifluoromethyl) phenyl)amino)ethyl)- 4,7-dihydro- [1,2,4]triazolo[1,5- a]pyrimidin-6-yl)- 4- methylphenyl)acryl amide | 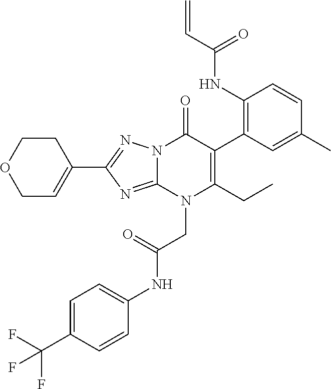 [SMILES: C=CC(=O)Nc1ccc(C)cc1-c1c(CC)n(CC(=O)Nc2ccc(C(F)(F)F)cc2)c2nc(C3=CCOCC3)nn2c1=O] | 607.3 | ¹H NMR (400 MHz, DMSO- d₆) δ 10.85 (s, 1H), 8.99 (s, 1H), 8.00 (d, J = 8.3 Hz, 1H), 7.80 (d, J = 8.5 Hz, 2H), 7.73 (d, J = 8.7 Hz, 2H), 7.23 (dd, J = 8.3, 2.1 Hz, 1H), 7.11-7.02 (m, 1H), 6.84 (s, 1H), 6.54-6.41 (m, 1H), 6.18 (dd, J = 17.0, 2.1 Hz, 1H), 5.66 (dd, J = 10.1, 2.2 Hz, 1H), 5.32- 5.17 (m, 2H), 4.27 (d, J = 2.9 Hz, 2H), 3.87- 3.76 (m, 2H), 2.54 (m, 3H), 2.33 (m, 4H), 0.94 (t, J = 7.4 Hz, 3H). |
| 10 | N-(5-Chloro-2-(2- (3,6-dihydro-2H- pyran-4-yl)-5- ethyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)acrylamide | 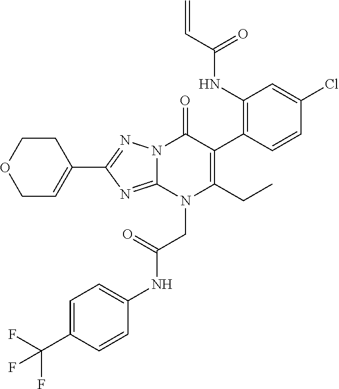 [SMILES: C=CC(=O)Nc1cc(Cl)ccc1-c1c(CC)n(CC(=O)Nc2ccc(C(F)(F)F)cc2)c2nc(C3=CCOCC3)nn2c1=O] | 627.2 | |
| 11 | N-(4-Chloro-2-(2- (3,6-dihydro-2H- pyran-4-yl)-5- ethyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)acrylamide | 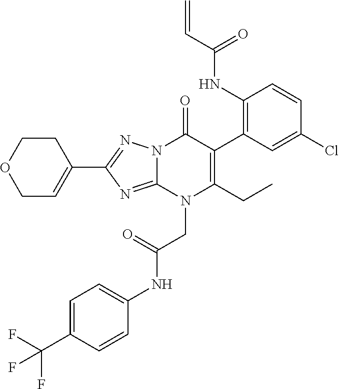 [SMILES: C=CC(=O)Nc1ccc(Cl)cc1-c1c(CC)n(CC(=O)Nc2ccc(C(F)(F)F)cc2)c2nc(C3=CCOCC3)nn2c1=O] | 627.2 | |
| 12 | N-(2-(2-(3,6- Dihydro-2H-pyran- 4-yl)-5-ethyl-7- oxo-4-(2-oxo-2- ((4- (trifluoromethyl) phenyl)amino)ethyl)- 4,7-dihydro- [1,2,4]triazolo[1,5- a]pyrimidin-6-yl)- 5- (trifluoromethyl) phenyl)acrylamide | 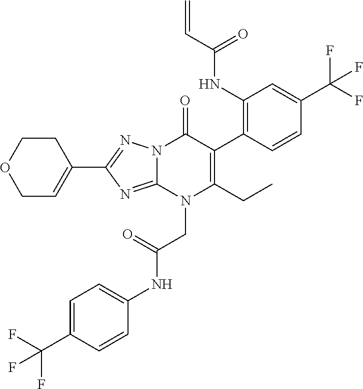 [SMILES: C=CC(=O)Nc1cc(C(F)(F)F)ccc1-c1c(CC)n(CC(=O)Nc2ccc(C(F)(F)F)cc2)c2nc(C3=CCOCC3)nn2c1=O] | 661.3 | |
| 13 | N-(2-(2-(3,6- Dihydro-2H-pyran- 4-yl)-5-ethyl-7- oxo-4-(2-oxo-2- ((4- (trifluoromethyl) phenyl)amino)ethyl)- 4,7-dihydro- [1,2,4]triazolo[1,5- a]pyrimidin-6-yl)- 5- fluorophenyl)acryl amide | 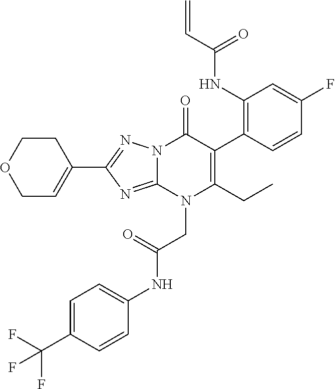 [SMILES: C=CC(=O)Nc1cc(F)ccc1-c1c(CC)n(CC(=O)Nc2ccc(C(F)(F)F)cc2)c2nc(C3=CCOCC3)nn2c1=O] | 611.2 | ¹H NMR (400 MHz, DMSO- d₆) δ 10.85 (s, 1H), 9.17 (s, 1H), 8.10 (dd, J = 11.8, 2.8 Hz, 1H), 7.80 (d, J = 8.6 Hz, 2H), 7.73 (d, J = 8.7 Hz, 2H), 7.29 (dd, J = 8.6, 6.6 Hz, 1H), 7.12- 7.05 (m, 1H), 6.85 (s, 1H), 6.52 (dd, J = 17.0, 10.1 Hz, 1H), 6.25 (dd, J = 17.0, 2.0 Hz, 1H), 5.74 (dd, J = 10.1, 2.0 Hz, 1H), 5.34-5.18 (m, 2H), 4.27 (d, J = 2.9 Hz, 2H), 3.85-3.78 (m, 2H), 2.54 (s, 2H), 2.31 (dd, J = 14.5, 7.3 Hz, 1H), 0.94 (t, J = 7.4 Hz, 3H). |

### Example 14. 2-(6-(1-Acryloyl-1,4,5,6-tetrahydropyridin-2-yl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

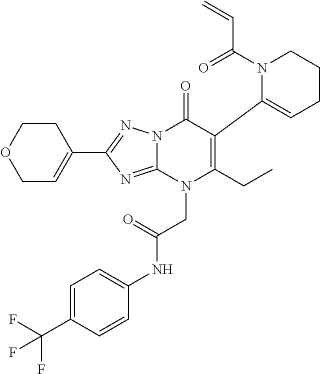 [SMILES: C=CC(=O)N1CCCC=C1c1c(CC)n(CC(=O)Nc2ccc(C(F)(F)F)cc2)c2nc(C3=CCOCC3)nn2c1=O]

### Step 1. tert-Butyl 6-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-3,4-dihydropyridine-1(2H)-carboxylate

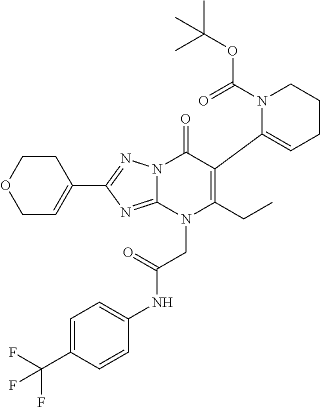 [SMILES: CCc1c(C2=CCCCN2C(=O)OC(C)(C)C)c(=O)n2nc(C3=CCOCC3)nc2n1CC(=O)Nc1ccc(C(F)(F)F)cc1]

2-(6-Bromo-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (76 mg, 0.144 mmol) (Example 1, Step 6), tert-butyl 6-(4,4,5,5-tetramethyl-1,3,2-dioxaborolan-2-yl)-3,4-dihydropyridine-1(2H)-carboxylate (134 mg, 0.433 mmol), XPhos Pd G4 (12.4 mg, 0.014 mmol) and potassium phosphate tribasic (77 mg, 0.361 mmol) were weighed into a vial. 1,4-Dioxane (1.5 mL) and water (0.3 mL) were added, purged with N₂, and heated to 90° C. After 3 h, more tert-butyl 6-(4,4,5,5-tetramethyl-1,3,2-dioxaborolan-2-yl)-3,4-dihydropyridine-1(2H)-carboxylate (134 mg, 0.433 mmol) was added and continued stirring overnight. After cooling, the mixture was filtered through a pad of celite, washing with EtOAc (5 mL), and concentrated. The crude was purified by flash column chromatography (4 g SiO₂, 5-100% EtOAc/Hex) to give 9 mg (10% yield, 30% purity) of the product. LC-MS calculated for C₃₁H₃₆F₃N₆O₅ (M+H)⁺: m/z=629.3; found 529.3 (M+H−Boc).

### Step 2. 2-(6-(1-Acryloyl-1,4,5,6-tetrahydropyridin-2-yl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

Trifluoroacetic acid (0.033 mL, 0.429 mmol) was added to a solution of tert-butyl 6-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-3,4-dihydropyridine-1(2H)-carboxylate (9 mg, 0.014 mmol) in DCM (0.12 mL) and stirred for 90 min. The solvents were evaporated. To the residue was added DCM (0.2 mL) and diisopropylethylamine (0.013 mL, 0.072 mmol) followed by acryloyl chloride (1.75 μl, 0.021 mmol). After 1 h, more acryloyl chloride (1.75 μl, 0.021 mmol) was added. After 1 h, added more diisopropylethylamine (6.5 uL). After 90 min, added more acryloyl chloride (1.6 uL) and heated to 30° C. After 30 min, the solvents were evaporated and the residue purified using preparatory LCMS (pH 2) to give 1.1 mg (13%) of the desired product. LC-MS calculated for C₂₆H₂₈F₃N₆O₃ (M+H)⁺: m/z=529.2; found 529.3 ([M−Boc+H]+H).

### Example 15. 2-(6-(1-Acryloylindolin-7-yl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

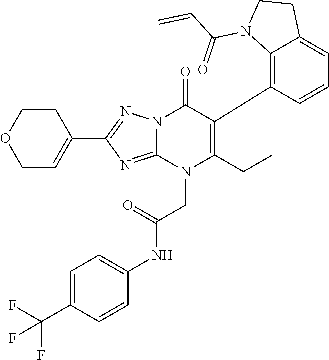 [SMILES: C=CC(=O)N1CCc2cccc(-c3c(CC)n(CC(=O)Nc4ccc(C(F)(F)F)cc4)c4nc(C5=CCOCC5)nn4c3=O)c21]

### Intermediate A. 2-Bromo-N-(4-(trifluoromethyl)phenyl)acetamide

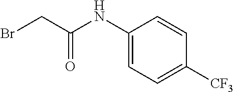 [SMILES: O=C(CBr)Nc1ccc(C(F)(F)F)cc1]

To a solution of 4-(Trifluoromethyl)aniline (0.779 mL, 6.21 mmol) in DCM (25 mL) stirring at about 0° C. was added bromoacetyl bromide (0.810 mL, 9.31 mmol) dropwise, followed by diisopropylethylamine (3.25 mL, 18.6 mmol). After 4 h, the reaction mixture was diluted with DCM and water. The mixture was transferred to a separatory funnel, layers separated and the organic was dried (MgSO₄), filtered and concentrated. The crude was purified using flash chromatography (0-55% EtOAc/Hex) to give the desired product as a light yellow solid (1.4 g, 80%). LC-MS calculated for C₉H₈BrF₃NO (M+H)⁺: m/z=282.0, 284.0; found 282.0, 284.0.

### Step 1. 2-(2-Bromo-5-ethyl-7-oxo-[1,2,4]triazolo[1,5-a]pyrimidin-4(7H)-yl)-N-(4-(trifluoromethyl)phenyl)acetamide

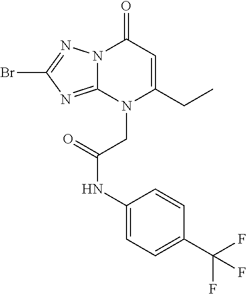 [SMILES: CCc1cc(=O)n2nc(Br)nc2n1CC(=O)Nc1ccc(C(F)(F)F)cc1]

To a mixture of 2-bromo-5-ethyl-[1,2,4]triazolo[1,5-a]pyrimidin-7(4H)-one (1.5 g, 6.17 mmol)(Example 1, Step 1) and 2-bromo-N-(4-(trifluoromethyl)phenyl)acetamide (2.09 g, 7.41 mmol) (Intermediate A) stirring in dimethylformamide (17 mL) was added diisopropylethylamine (3.23 mL, 18.5 mmol). The resulting mixture was heated to 55° C. and stirred for 6 h, then cooled down to room temperature and continued to stir for overnight. The flask was submerged in an ice bath and excess water was added. Stirred for 60 min. The resulting solids were filtered and dried to give 2.4 g of the crude product. Excess DCM (20 mL) was added to the crude solids, stirred for 15 min, then filtered and dried to give 1.96 g (71.5%) of the desired product as a light yellow amorphous solid. LC-MS calculated for C₁₆H₁₄BrF₃N₅O₂ (M+H)⁺: m/z=444.0, 446.0; found 444.0, 446.0.

### Step 2. 2-(2-(3,6-Dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

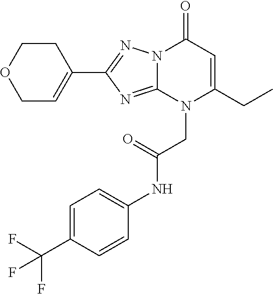 [SMILES: CCc1cc(=O)n2nc(C3=CCOCC3)nc2n1CC(=O)Nc1ccc(C(F)(F)F)cc1]

Into a round-bottom flask was added 2-(2-bromo-5-ethyl-7-oxo-[1,2,4]triazolo[1,5-a]pyrimidin-4(7H)-yl)-N-(4-(trifluoromethyl)phenyl)acetamide (1.96 g, 4.41 mmol), 2-(3,6-dihydro-2H-pyran-4-yl)-4,4,5,5-tetramethyl-1,3,2-dioxaborolane (1.39 g, 6.62 mmol), chloro(2-dicyclohexylphosphino-2′,4′,6′-triisopropyl-1,1′-biphenyl)[2-(2′-amino-1,1′-biphenyl)]palladium(II) (0.347 g, 0.441 mmol), and potassium phosphate tribasic (2.34 g, 11.0 mmol). The flask was flushed with N₂ while adding 1,4-dioxane (30 mL)/water (6 mL). The mixture was stirred at 80° C. for 2 h. After cooling, filtered through celite, washing with EtOAc. The filtrate was washed with brine, dried (Na₂SO₄), filtered and concentrated to give a solid mixture. The crude solid was partially dissolved in DCM, and methyl tert-butyl ether was added. The solvents were evaporated and to the solids was added DCM (15-20 mL) followed by methyl tert-butyl ether (˜50 mL), with stirring. Stirred briefly, filtered and dried in vacuo to give 0.90 g (46%) of the desired product as a light-yellow amorphous solid. LC-MS calculated for C₂₁H₂₁F₃N₅O₃ (M+H)⁺: m/z=448.2; found 448.2.

### Step 3. 2-(6-Bromo-2-(3,6-dihydro-2H-pyran-4-yl)-5-ethyl-7-oxo-[1, 2,4]triazolo[1,5-a]pyrimidin-4(7H)-yl)-N-(4-(trifluoromethyl)phenyl)acetamide

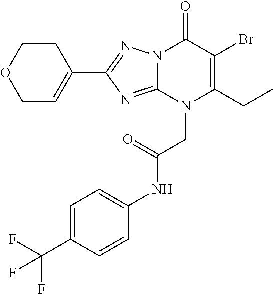 [SMILES: CCc1c(Br)c(=O)n2nc(C3=CCOCC3)nc2n1CC(=O)Nc1ccc(C(F)(F)F)cc1]

To a mixture of 2-(2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (0.78 g, 1.74 mmol) in acetonitrile (10 mL) was added N-bromosuccinimide (0.341 g, 1.92 mmol). Stirred at room temperature overnight. LCMS showed the reaction was only 50% complete. Added more N-bromosuccinimide (0.341 g, 1.92 mmol) and stirred for 2 h. Water was added then extracted with DCM (10 mL×3). The combined extracts were dried (MgSO₄), filtered and concentrated. The crude was purified by flash column chromatography (40 g SiO₂, 5-100% EtOAc/Hex) to give 0.43 g desired product (47%) as a white amorphous solid. LC-MS calculated for C₂₁H₂₀BrF₃N₅O₃ (M+H)⁺: m/z=526.1, 528.1; found 526.1, 528.1.

### Step 4. 2-(2-(3,6-Dihydro-2H-pyran-4-yl)-5-ethyl-6-(indolin-7-yl)-7-oxo-[1, 2,4]triazolo[1,5-a]pyrimidin-4(7H)-yl)-N-(4-(trifluoromethyl)phenyl)acetamide

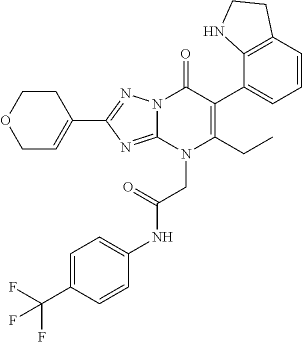 [SMILES: CCc1c(-c2cccc3c2NCC3)c(=O)n2nc(C3=CCOCC3)nc2n1CC(=O)Nc1ccc(C(F)(F)F)cc1]

Into a small vial were weighed 2-(6-bromo-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide (21 mg, 0.040 mmol), tert-butyl 7-(4,4,5,5-tetramethyl-1,3,2-dioxaborolan-2-yl)indoline-1-carboxylate (20.7 mg, 0.060 mmol), XPhos Pd G4 (3.43 mg, 3.99 μmol) and potassium carbonate (16.5 mg, 0.120 mmol). 1,4-Dioxane (0.3 mL) and water (60 μL) were added, purged with N₂, and heated to 90° C. for 18 h. After cooling, the mixture was filtered through a Si-thiol cartridge, eluting with DCM (2 mL) and a small amount of MeOH (0.5 mL). The eluent was concentrated and dried in vacuo. LCMS revealed that the Boc group was removed during the reaction. The product was used for the next step without purification. Assumed quantitative yield. LC-MS calculated for C₂₉H₂₈F₃N₆O₃ (M+H)⁺: m/z=565.2; found 565.2.

### Step 5. 2-(6-(1-Acryloylindolin-7-yl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide

To a solution of 2-(2-(3,6-dihydro-2H-pyran-4-yl)-5-ethyl-6-(indolin-7-yl)-7-oxo-[1,2,4]triazolo[1,5-a]pyrimidin-4(7H)-yl)-N-(4-(trifluoromethyl)phenyl)acetamide (12 mg, 0.021 mmol) in DCM (0.40 mL) stirring at r.t. was added diisopropylethylamine (0.019 mL, 0.106 mmol), then acryloyl chloride (2.59 μl, 0.032 mmol). The mixture was stirred for 90 min. then the solvents evaporated. The crude was purified using preparatory LCMS (pH 2) to give 3 mg (23%) of the desired product. LC-MS calculated for C₃₂H₃₀F₃N₆O₄ (M+H)⁺: m/z=619.2; found 619.3.

### Example 16. N-(2-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)phenyl)acrylamide

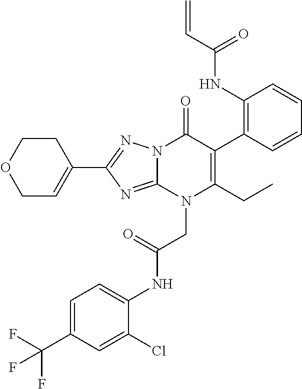 [SMILES: C=CC(=O)Nc1ccccc1-c1c(CC)n(CC(=O)Nc2ccc(C(F)(F)F)cc2Cl)c2nc(C3=CCOCC3)nn2c1=O]

### Step 1. tert-Butyl 2-(6-bromo-2-(3,6-dihydro-2H-pyran-4-yl)-5-ethyl-7-oxo-[1,2,4]triazolo[1, 5-a]pyrimidin-4(7H)-yl)acetate

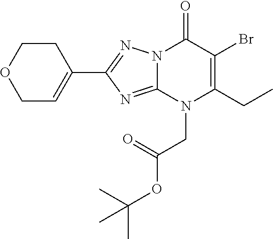 [SMILES: CCc1c(Br)c(=O)n2nc(C3=CCOCC3)nc2n1CC(=O)OC(C)(C)C]

To a mixture of tert-butyl 2-(2-(3,6-dihydro-2H-pyran-4-yl)-5-ethyl-7-oxo-[1,2,4]triazolo[1,5-a]pyrimidin-4(7H)-yl)acetate (1.3 g, 3.61 mmol) (Example 1, Step 3) in acetonitrile (30 mL) was added N-bromosuccinimide (0.706 g, 3.97 mmol). Stirred at room temperature overnight. Water was added then extracted with DCM (30 mL×3). The combined extracts were run through a phase separator and concentrated. The crude was purified by flash column chromatography (40 g SiO₂, 0-70% EtOAc/Hex) to give 0.87 g (55%) of the product as white amorphous solids. LC-MS calculated for C₁₈H₂₄BrN₄O₄ (M+H)⁺: m/z=439.1, 441.1; found 439.1, 441.1.

### Step 2. 2-(6-Bromo-2-(3,6-dihydro-2H-pyran-4-yl)-5-ethyl-7-oxo-[1, 2,4]triazolo[1,5-a]pyrimidin-4(7H)-yl)acetic acid

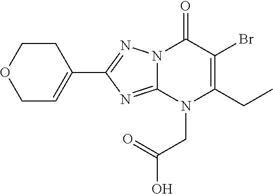 [SMILES: CCc1c(Br)c(=O)n2nc(C3=CCOCC3)nc2n1CC(=O)O]

To a mixture of tert-butyl 2-(6-bromo-2-(3,6-dihydro-2H-pyran-4-yl)-5-ethyl-7-oxo-[1,2,4]triazolo[1,5-a]pyrimidin-4(7H)-yl)acetate (0.87 g, 1.98 mmol) in DCM (6 mL) was added Trifluoroacetic acid (3.05 mL, 39.6 mmol). The resulting solution was stirred at room temperature overnight. The solvents were evaporated and the residue dried in vacuo to give the desired product in quantitative yield. LC-MS calculated for C₁₄H₁₆BrN₄O₄ (M+H)⁺: m/z=383.0, 385.0; found 383.0, 385.0.

### Step 3. 2-(6-Bromo-2-(3,6-dihydro-2H-pyran-4-yl)-5-ethyl-7-oxo-[1, 2,4]triazolo[1,5-a]pyrimidin-4(7H)-yl)-N-(2-chloro-4-(trifluoromethyl)phenyl)acetamide

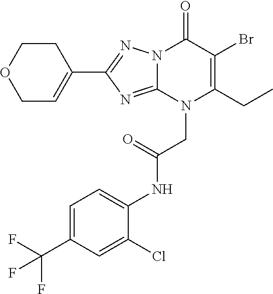 [SMILES: CCc1c(Br)c(=O)n2nc(C3=CCOCC3)nc2n1CC(=O)Nc1ccc(C(F)(F)F)cc1Cl]

To a mixture of 2-(6-bromo-2-(3,6-dihydro-2H-pyran-4-yl)-5-ethyl-7-oxo-[1,2,4]triazolo[1,5-a]pyrimidin-4(7H)-yl)acetic acid (102 mg, 0.266 mmol), 2-chloro-4-(trifluoromethyl)aniline (57.3 mg, 0.293 mmol), and triethylamine (0.148 mL, 1.07 mmol) in acetonitrile (2 mL) was added n-propylphosphonic acid anhydride, cyclic trimer (0.471 mL, 0.799 mmol). Stirred at room temperature overnight (23 hrs). The reaction mixture was partitioned between water (5 mL) and EtOAc (10 mL). The layers were separated and the aqueous extracted with EtOAc (10 mL). Combined organics were washed with saturated aq. sodium bicarbonate, water and brine, then dried (Na₂SO₄), filtered and concentrated. The crude was purified by flash column chromatography (12 g SiO₂,20-100% EtOAc/Hex) to give 34 mg (23%) of the desired product. LC-MS calculated for C₂₁H₁₉BrClF₃N₅O₃ (M+H)⁺: m/z=560.0, 562.0; found 560.0, 562.0.

### Step 4. 2-(6-(2-Aminophenyl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

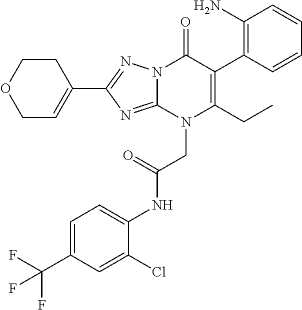 [SMILES: CCc1c(-c2ccccc2N)c(=O)n2nc(C3=CCOCC3)nc2n1CC(=O)Nc1ccc(C(F)(F)F)cc1Cl]

2-(6-Bromo-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide (34 mg, 0.061 mmol), (2-aminophenyl)boronic acid (8.30 mg, 0.061 mmol), XPhos Pd G4 (5.22 mg, 6.06 μmol) and potassium phosphate tribasic (32.2 mg, 0.152 mmol) were weighed into a vial. 1,4-Dioxane (0.6 mL) and water (120 μL) were added, purged with N₂, and heated to 70° C. for 4 h. After cooling, the mixture was filtered through a pad of celite and concentrated. The crude was purified using preparatory LCMS (pH 2) to give 13 mg (37%) of the desired product. LC-MS calculated for C₂₇H₂₅ClF₃N₆O₃ (M+H)⁺: m/z=573.2; found 573.2.

### Step 5. N-(2-(4-(2-((2-Chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)phenyl)acrylamide

A solution of 2-(6-(2-aminophenyl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide (11 mg, 0.019 mmol) in DCM (0.30 mL) was cooled in an ice bath to about 0° C. before adding diisopropylethylamine (10.1 μl, 0.058 mmol), then acryloyl chloride (2.34 μl, 0.029 mmol). The mixture was warmed to r.t. and stirred overnight. The solvents were evaporated and the residue purified using preparatory LCMS (pH 2) to give 3.4 mg (28%) of the desired product. LC-MS calculated for C₃₀H₂₇ClF₃N₆O₄ (M+H)⁺: m/z=627.2; found 627.2. ¹H NMR (400 MHz, DMSO-d₆) δ 10.30 (s, 1H), 9.05 (s, 1H), 8.17-8.06 (m, 2H), 7.98 (d, J=2.1 Hz, 1H), 7.75 (dd, J=8.7, 2.2 Hz, 1H), 7.45-7.41 (m, 1H), 7.24 (d, J=4.5 Hz, 2H), 6.89 (s, 1H), 6.46 (dd, J=17.0, 10.2 Hz, 1H), 6.20 (dd, J=17.0, 2.1 Hz, 1H), 5.66 (dd, J=10.1, 2.1 Hz, 1H), 5.37 (d, J=3.0 Hz, 2H), 4.32-4.26 (m, 2H), 3.87-3.78 (m, 2H), 2.56 (s, 2H), 2.39-2.29 (m, 1H), 0.96 (t, J=7.4 Hz, 3H).

### Example 17. N-(2-(2-Cyclopropyl-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide

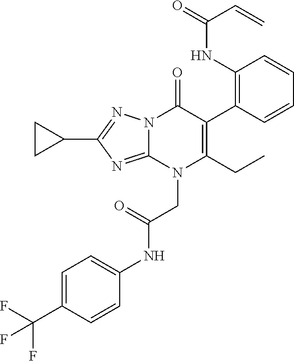 [SMILES: C=CC(=O)Nc1ccccc1-c1c(CC)n(CC(=O)Nc2ccc(C(F)(F)F)cc2)c2nc(C3CC3)nn2c1=O]

### Step 1: 2-Cyclopropyl-5-ethyl-[1,2,4]triazolo[1,5-a]pyrimidin-7(4H)-one

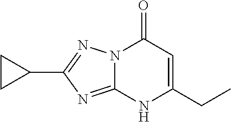

A mixture of 3-cyclopropyl-1H-1,2,4-triazol-5-amine (1.00 g, 8.05 mmol) and ethyl propionylacetate (2.41 mL, 16.9 mmol) in acetic acid (6.0 mL) was stirred at 80° C. overnight (24 hrs). After cooling, ice (10 g) was added. The solids were filtered, washed with a cold water (3.0 mL), and dried to give 1.1 g (67%) of product. LCMS calculated for C₁₀H₁₃N₄O (M+H)⁺: m/z=205.1, found=205.1.

### Step 2: 2-(2-Cyclopropyl-5-ethyl-7-oxo-[1, 2,4]triazolo[1, 5-a]pyrimidin-4(7H)-yl)-N-(4-(trifluoromethyl)phenyl)acetamide

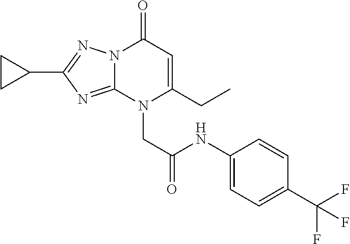 [SMILES: CCc1cc(=O)n2nc(C3CC3)nc2n1CC(=O)Nc1ccc(C(F)(F)F)cc1]

To the stirred solution of 2-cyclopropyl-5-ethyl-[1,2,4]triazolo[1,5-a]pyrimidin-7(4H)-one (0.46 g, 2.25 mmol) and 2-bromo-N-(4-(trifluoromethyl)phenyl)acetamide (Intermediate A) (0.76 g, 2.70 mmol) in DMF (3 mL) was added N-ethyl-N-isopropylpropan-2-amine (1.18 mL, 6.76 mmol). The resulting mixture was heated to 55° C. (a clear solution formed), and stirred for 6 h. Cooled to room temperature, then ice (10 g) was added, stirred for 1 h, filtered and dried in vacuo to give the desired product 2-(2-cyclopropyl-5-ethyl-7-oxo-[1,2,4]triazolo[1,5-a]pyrimidin-4(7H)-yl)-N-(4-(trifluoromethyl)phenyl)acetamide along with its regioisomer (2-(2-cyclopropyl-5-ethyl-7-oxo-[1,2,4]triazolo[1,5-a]pyrimidin-3(7H)-yl)-N-(4-(trifluoromethyl)phenyl)acetamide) as a white solid (430 mg, 47%). LC-MS calculated for C₁₉H₁₉F₃N₅O₂ (M+H)⁺: m/z=406.1; found 406.1.Step 3: 2-(6-Bromo-2-cyclopropyl-5-ethyl-7-oxo-[1,2,4]triazolo[1,5-a]pyrimidin-4(7H)-yl)-N-(4-(trifluoromethyl)phenyl)acetamide

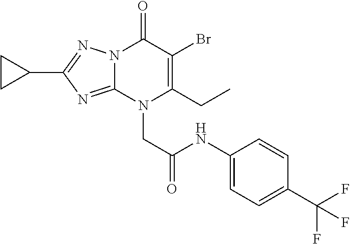 [SMILES: CCc1c(Br)c(=O)n2nc(C3CC3)nc2n1CC(=O)Nc1ccc(C(F)(F)F)cc1]

To a mixture of 2-(2-cyclopropyl-5-ethyl-7-oxo-[1,2,4]triazolo[1,5-a]pyrimidin-4(7H)-yl)-N-(4-(trifluoromethyl)phenyl)acetamide (0.86 g, 2.12 mmol) in DMF (4.5 mL) was added N-bromosuccinimide (0.38 g, 2.12 mmol) at room temperature. The reaction mixture was heated to 55° C. and stirred for 6 h. After completion of the reaction, reaction mixture was cooled to room temperature and ice (10 g) was added, stirred for 10 min, filtered and dried in vacuo. The crude was purified using flash column chromatography (40 g SiO₂, 5-100% EtOAc/Hex) to give the desired product 2-(6-bromo-2-cyclopropyl-5-ethyl-7-oxo-[1,2,4]triazolo[1,5-a]pyrimidin-4(7H)-yl)-N-(4-(trifluoromethyl)phenyl)acetamide along with its regioisomer (2-(6-bromo-2-cyclopropyl-5-ethyl-7-oxo-[1,2,4]triazolo[1,5-a]pyrimidin-3(7H)-yl)-N-(4-(trifluoromethyl)phenyl)acetamide) as a pale yellow solid (822 mg, 80%). LC-MS calculated for C₁₉H₁₈BrF₃N₅O₂ (M+H)⁺: m/z=484.1; found 484.1.Step 4: 2-(6-(2-Aminophenyl)-2-cyclopropyl-5-ethyl-7-oxo-[1,2,4]triazolo[1,5-a]pyrimidin-4(7H)-yl)-N-(4-(trifluoromethyl)phenyl)acetamide

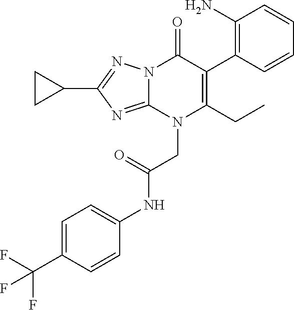 [SMILES: CCc1c(-c2ccccc2N)c(=O)n2nc(C3CC3)nc2n1CC(=O)Nc1ccc(C(F)(F)F)cc1]

2-(6-Bromo-2-cyclopropyl-5-ethyl-7-oxo-[1,2,4]triazolo[1,5-a]pyrimidin-4(7H)-yl)-N-(4-(trifluoromethyl)phenyl)acetamide (100 mg, 0.206 mmol), (2-aminophenyl)boronic acid (56.6 mg, 0.413 mmol), XPhos Pd G4 (17.77 mg, 0.02 mmol) and potassium phosphate tribasic (110 mg, 0.52 mmol) were weighed into a 2 dram vial. 1,4-Dioxane (1.0 mL) and water (200 μL) were added, purged with N₂, and heated to 80° C. for 1 h. After cooling, the mixture was filtered through a pad of celite, washing with EtOAc (10 mL), and concentrated. The crude was purified by flash column chromatography (12 g SiO₂, 5-100% EtOAc/Hex) to give the desired product 2-(6-(2-aminophenyl)-2-cyclopropyl-5-ethyl-7-oxo-[1,2,4]triazolo[1,5-a]pyrimidin-4(7H)-yl)-N-(4-(trifluoromethyl)phenyl)acetamide which eluted around 90% EtOAc/Hex (58 mg, 57%). LCMS calculated for C₂₅H₂₄F₃N₆O₂ (M+H)⁺: m/z=497.2.2; found 497.2. ¹H NMR (500 MHz, DMSO-d₆) δ 10.75 (s, 1H), 7.79 (d, J=8.5 Hz, 2H), 7.72 (d, J=8.6 Hz, 2H), 7.11-7.06 (m, 1H), 6.90 (dd, J=7.5, 1.6 Hz, 1H), 6.72 (d, J=8.1 Hz, 1H), 6.60 (t, J=7.4 Hz, 1H), 5.23-5.10 (m, 2H), 4.88 (s, 2H), 2.62-2.52 (m, 1H), 2.45-2.35 (m, 1H), 2.08-2.03 (m, 1H), 1.03-0.97 (m, 5H), 0.92-0.86 (m, 2H).

A second product which eluted at around 100% EtOAc was consistent with the regioisomer (2-(6-(2-aminophenyl)-2-cyclopropyl-5-ethyl-7-oxo-[1,2,4]triazolo[1,5-a]pyrimidin-3(7H)-yl)-N-(4-(trifluoromethyl)phenyl)acetamide). LCMS calculated for C₂₅H₂₄F₃N₆O₂ (M+H)⁺: m/z=497.2.2; found 497.2.

### Step 5: N-(2-(2-Cyclopropyl-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide

To a solution of 2-(6-(2-aminophenyl)-2-cyclopropyl-5-ethyl-7-oxo-[1,2,4]triazolo[1,5-a]pyrimidin-4(7H)-yl)-N-(4-(trifluoromethyl)phenyl)acetamide (0.015 g, 0.030 mmol) in DCM (0.5 mL) at 0° C. was added N-ethyl-N-isopropylpropan-2-amine (3.90 mg, 0.030 mmol) followed by acryloyl chloride (2.73 mg, 0.03 mmol). Reaction was stirred at same temperature for 15 min and then quenched with saturated NaHCO₃ (1 mL), followed by extraction using DCM. Organic solvents were evaporated and the residue purified using preparatory LCMS (pH 2) to give the desired product (8 mg, 48%) as a white solid. LC-MS calculated for C₂₈H₂₆F₃N₆O₃ (M+H)⁺: m/z=551.2; found 551.2. ¹H NMR (500 MHz, DMSO-d₆) δ 10.83 (s, 1H), 9.05 (s, 1H), 8.12 (d, J=8.2 Hz, 1H), 7.80 (d, J=8.6 Hz, 2H), 7.73 (d, J=8.6 Hz, 2H), 7.42 (ddd, J=8.6, 5.7, 3.3 Hz, 1H), 7.25-7.20 (m, 2H), 6.50 (dd, J=17.0, 10.2 Hz, 1H), 6.20 (dd, J=17.0, 2.0 Hz, 1H), 5.69 (dd, J=10.2, 2.1 Hz, 1H), 5.30-5.10 (m, 2H), 2.50-2.41 (m, 1H), 2.35-2.30 (m, 1H), 2.10-2.06 (m, 1H), 1.06-0.99 (m, 2H), 0.95-0.91 (m, 5H).

### Examples 18-23

Example compounds 18-23 in Table 3 were prepared according to the procedures described in Example 17, using appropriately substituted starting materials.

TABLE 3

| Ex. | | | LCMS | |
|---|---|---|---|---|
| No. | Name | Structure | [M + H]⁺ | ¹H NMR |
| 18 | N-(2-(5-Ethyl-2- methyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)acrylamide | 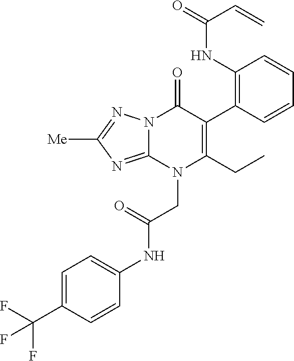 [SMILES: C=CC(=O)Nc1ccccc1-c1c(CC)n(CC(=O)Nc2ccc(C(F)(F)F)cc2)c2nc(C)nn2c1=O] | 525.2 | ¹H NMR (400 MHz, DMSO- d₆) δ 10.84 (s, 1H), 9.07 (s, 1H), 8.12 (d, J = 8.2 Hz, 1H), 7.80 (d, J = 8.6 Hz, 2H), 7.73 (d, J = 8.7 Hz, 2H), 7.45-7.40 (m, 1H), 7.24 (d, J = 4.4 Hz, 2H), 6.51 (dd, J = 17.5, 9.6 Hz, 1H), 6.20 (dd, J = 17.0, 2.1 Hz, 1H), 5.69 (dd, J = 10.2, 2.1 Hz, 1H), 5.27 (d, J = 17.3 Hz, 1H), 5.18 (d, J = 17.3 Hz, 1H), 2.49- 2.42 (m, 1H), 2.39 (s, 3H), 2.36-2.30 (m, 1H), 0.93 (t, J = 7.4 Hz, 3H). |
| 19 | N-(2-(2- Cyclobutyl-5- ethyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)acrylamide | 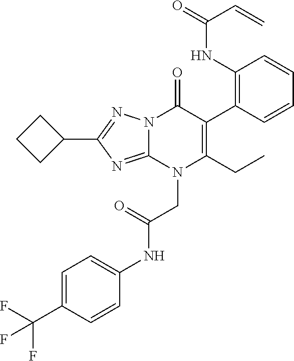 [SMILES: C=CC(=O)Nc1ccccc1-c1c(CC)n(CC(=O)Nc2ccc(C(F)(F)F)cc2)c2nc(C3CCC3)nn2c1=O] | 565.2 | ¹H NMR (400 MHz, DMSO- d₆) δ 10.83 (s, 1H), 9.07 (s, 1H), 8.13 (d, J = 8.2 Hz, 1H), 7.80 (d, J = 8.6 Hz, 2H), 7.73 (d, J = 8.6 Hz, 2H), 7.46-7.38 (m, 1H), 7.27-7.20 (m, 2H), 6.51 (dd, J = 17.0, 10.2 Hz, 1H), 6.20 (dd, J = 17.0, 2.1 Hz, 1H), 5.69 (dd, J = 10.1, 2.1 Hz, 1H), 5.24 (q, J = 17.3 Hz, 2H), 3.68-3.62 (m, 1H), 2.52-2.42 (m, 1H), 2.39- 2.28 (m, 5H), 2.12-1.97 (m, 1H), 1.92 (dq, J = 11.3, 6.3 Hz, 1H), 0.92 (t, J = 7.4 Hz, 3H). |
| 20 | N-(2-(2- Cyclohexyl-5- ethyl-7-oxo-4-(2- oxo-2-((4- (trifluoromethyl) phenyl)amino)ethyl)- 4,7-dihydro- [1,2,4]triazolo[1,5- a]pyrimidin-6- yl)phenyl)acrylamide | 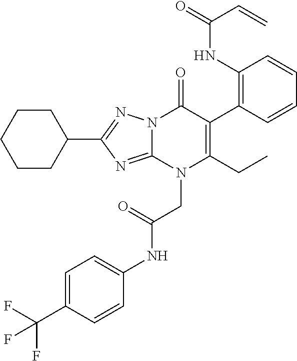 [SMILES: C=CC(=O)Nc1ccccc1-c1c(CC)n(CC(=O)Nc2ccc(C(F)(F)F)cc2)c2nc(C3CCCCC3)nn2c1=O] | 593.3 | ¹H NMR (600 MHz, DMSO- d₆) δ 10.83 (s, 1H), 9.10 (s, 1H), 8.13 (d, J = 8.2 Hz, 1H), 7.80 (d, J = 8.6 Hz, 2H), 7.73 (d, J = 8.6 Hz, 2H), 7.42 (ddd, J = 8.6, 6.2, 2.9 Hz, 1H), 7.24-7.21 (m, 2H), 6.51 (dd, J = 17.0, 10.2 Hz, 1H), 6.20 (dd, J = 17.0, 2.0 Hz, 1H), 5.69 (dd, J = 10.2, 2.0 Hz, 1H), 5.26 (d, J = 17.3 Hz, 1H), 5.19 (d, J = 17.3 Hz, 1H), 2.78- 2.73 (m, 1H), 2.48-2.42 (m, 1H), 2.35-2.30 (m, 1H), 1.97 (d, J = 12.8 Hz, 2H), 1.81-1.74 (m, 2H), 1.71- 1.65 (m, 1H), 1.55-1.50 (m, 2H), 1.36 (q, J = 12.7 Hz, 2H), 1.28-1.22 (m, 1H), 0.91 (t, J = 7.5 Hz, 3H). |
| 21 | N-(2-(4-(2-((2- Chloro-4- (trifluoromethyl) phenyl)amino)-2- oxoethyl)-5-ethyl- 2-isopropyl-7-oxo- 4,7-dihydro- [1,2,4]triazolo[1,5- a]pyrimidin-6- yl)phenyl)acrylamide | 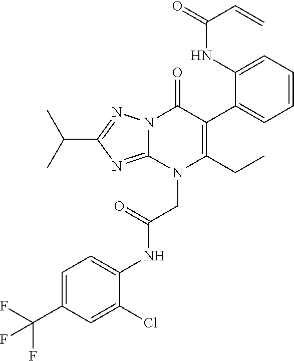 [SMILES: C=CC(=O)Nc1ccccc1-c1c(CC)n(CC(=O)Nc2ccc(C(F)(F)F)cc2Cl)c2nc(C(C)C)nn2c1=O] | 587.2 | |
| 22 | N-(2-(5-Ethyl-7- oxo-4-(2-oxo-2- ((4- (trifluoromethyl) phenyl)amino)ethyl)- 2-(thiophen-2-yl)- 4,7-dihydro- [1,2,4]triazolo[1,5- a]pyrimidin-6- yl)phenyl)acrylamide | 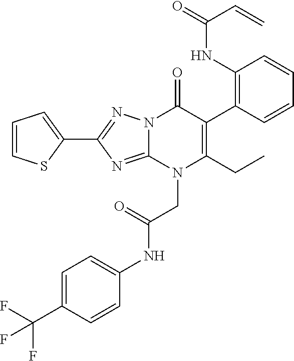 [SMILES: C=CC(=O)Nc1ccccc1-c1c(CC)n(CC(=O)Nc2ccc(C(F)(F)F)cc2)c2nc(-c3cccs3)nn2c1=O] | 593.2 | ¹H NMR (500 MHz, DMSO- d₆) δ 10.90 (s, 1H), 9.10 (s, 1H), 8.13 (d, J = 8.2 Hz, 1H), 7.82 (d, J = 8.6 Hz, 2H), 7.77 (ddd, J = 9.2, 4.4, 1.2 Hz, 2H), 7.73 (d, J = 8.6 Hz, 2H), 7.44 (ddd, J = 8.6, 6.7, 2.3 Hz, 1H), 7.30-7.23 (m, 2H), 7.22 (dd, J = 5.0, 3.6 Hz, 1H), 6.51 (dd, J = 17.0, 10.2 Hz, 1H), 6.21 (dd, J = 17.0, 2.0 Hz, 1H), 5.69 (dd, J = 10.2, 2.0 Hz, 1H), 5.34 (d, J = 17.4 Hz, 1H), 5.26 (d, J = 17.3 Hz, 1H), 2.53- 2.41 (m, 1H), 2.39-2.33 (m, 1H), 0.95 (t, J = 7.5 Hz, 3H). |
| 23 | N-(2-(5-Ethyl-7- oxo-4-(2-oxo-2- ((4- (trifluoromethyl) phenyl)amino)ethyl)- 2-(tetrahydro-2H- pyran-4-yl)-4,7- dihydro- [1,2,4]triazolo[1,5- a]pyrimidin-6- yl)phenyl)acrylamide | 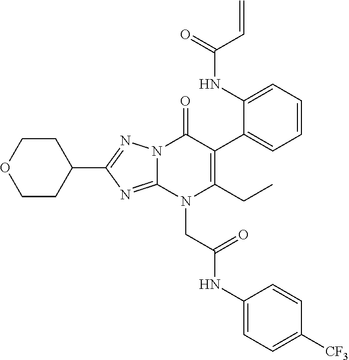 [SMILES: C=CC(=O)Nc1ccccc1-c1c(CC)n(CC(=O)Nc2ccc(C(F)(F)F)cc2)c2nc(C3CCOCC3)nn2c1=O] | 595.2 | |

### Example 24. N-(2-(5-Ethyl-7-oxo-4-(2-oxo-2-((4-(trifluoromethyl)phenyl)amino)ethyl)-2-(piperidin-4-yl)-4,7-dihydro-[1,2,4]triazolo[1,5-a]pyrimidin-6-yl)phenyl)acrylamide

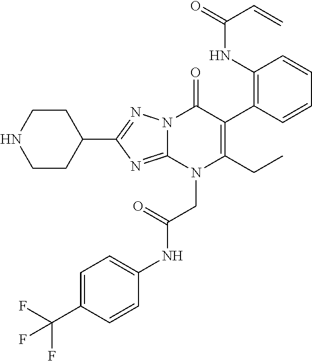 [SMILES: C=CC(=O)Nc1ccccc1-c1c(CC)n(CC(=O)Nc2ccc(C(F)(F)F)cc2)c2nc(C3CCNCC3)nn2c1=O]

### Step 1: tert-Butyl 4-(6-(2-acrylamidophenyl)-5-ethyl-7-oxo-4-(2-oxo-2-((4-(trifluoromethyl)phenyl)amino)ethyl)-4,7-dihydro-[1,2,4]triazolo[1,5-a]pyrimidin-2-yl)piperidine-1-carboxylate

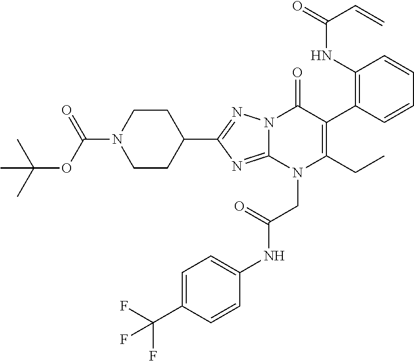 [SMILES: C=CC(=O)Nc1ccccc1-c1c(CC)n(CC(=O)Nc2ccc(C(F)(F)F)cc2)c2nc(C3CCN(C(=O)OC(C)(C)C)CC3)nn2c1=O]

The compound was prepared according to the procedures described in Example 17, using 1,1-dimethylethyl 45-amino-H-1,2,4-triazol-3-yl)-1-piperidinecarboxylate (CAS: 1429217-58-2, Enamine, EN300-7472905) as substituted starting materials. LC-MS calculated for C₃₅H₃₉F₃N₇O₅ (M+H)⁺: m/z=694.2; found 694.3.

### Step 2: N-(2-(5-Ethyl-7-oxo-4-(2-oxo-2-((4-(trifluoromethyl)phenyl)amino)ethyl)-2-(piperidin-4-yl)-4,7-dihydro-[1,2,4]triazolo[1,5-a]pyrimidin-6-yl)phenyl)acrylamide

To a stirred solution of tert-butyl 4-(6-(2-acrylamidophenyl)-5-ethyl-7-oxo-4-(2-oxo-2-((4-(trifluoromethyl)phenyl)amino)ethyl)-4,7-dihydro-[1,2,4]triazolo[1,5-a]pyrimidin-2-yl)piperidine-1-carboxylate (10 mg, 0.016 mmol) in DCM (0.5 mL) was added 2,2,2-trifluoroacetic acid (0.024 mL, 0.31 mmol) at room temperature. The reaction mixture was stirred for 30 min at same temperature. Reaction mixture was concentrated in vacuo and the residue purified using preparatory LCMS (pH 2) to give the desired product 4 mg (47%) as a white solid. LC-MS calculated for C₃₀H₃₁F₃N₇O₃ (M+H)⁺: m/z=594.2; found 594.3.

### Example 25. N-(2-(2-(1-Acryloylpiperidin-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide

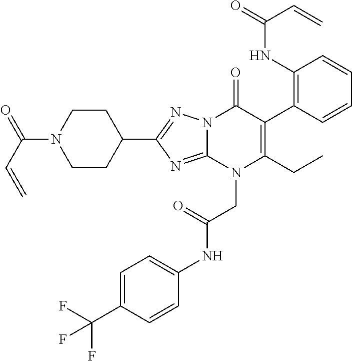 [SMILES: C=CC(=O)Nc1ccccc1-c1c(CC)n(CC(=O)Nc2ccc(C(F)(F)F)cc2)c2nc(C3CCN(C(=O)C=C)CC3)nn2c1=O]

To a solution of N-(2-(5-ethyl-7-oxo-4-(2-oxo-2-((4-(trifluoromethyl)phenyl)amino)ethyl)-2-(piperidin-4-yl)-4,7-dihydro-[1,2,4]triazolo[1,5-a]pyrimidin-6-yl)phenyl)acrylamide (Example 24) (0.010 g, 0.016 mmol) in DCM (0.5 mL) at 0° C. was added N-ethyl-N-isopropylpropan-2-amine (2.2 mg, 0.030 mmol) followed by acryloyl chloride (1.5 mg, 0.030 mmol). Reaction was stirred at same temperature for 15 min and then quenched with saturated NaHCO₃ (1 mL), followed by extraction using DCM. Organic solvent was evaporated and the residue purified using preparatory LCMS (pH 2) to give the desired product 5 mg (46%) as a white solid. LC-MS calculated for C₃₃H₃₃F₃N₇O₄ (M+H)+: m/z=648.3; found 648.3.

### Example A. WRN ATPase Activity Assay

Compounds of the Examples were tested for WRN ATPase activity. WRN ATPase activity catalyzes ATP hydrolysis to produce ADP and phosphate. DNA encoding the target protein, 6×HIS-TEV-WRN-517-945, was produced, cloned and transfected as detailed in Example D. The amount of ADP produced was measured using ADP-Glo™ Kinase Assay kit from Promega Corporation in a Greiner white regular volume 384 wells assay plate (Greiner #781075). Data was normalized to a standard curve generated using various ADP concentrations and the corresponding luminescent signal intensities at each concentration. The assay conditions were optimized for enzyme concentration (WRN), substrate concentrations (ATP & dsDNA), and the linearity of the ATPase reaction, in the following assay buffer: 50 mM Tris-HCl, pH-7.5, 2 mM MgCl₂, 50 mM NaCl, 1 mM EGTA, 0.01% P-20, 0.05% BSA, and 2 mM DTT prepared with nuclease free water.

A serial dilution (1:3 scheme) of the compounds were prepared in neat DMSO, with 100 nL of the compounds dispensed in the 384 wells assay plate. An 1-hr pre-incubation of the compounds with enzyme was included. A His-tagged construct of WRN (AA S2-S1235) was produced and used for the ATPase assay. Maximum inhibition control wells were set up with the addition of only assay buffer, and no inhibition control wells were set up with neat DMSO without test compounds. The reaction was initiated by addition of 5 μL substrate mix (200 μM ultrapure ATP, and 50 nM dsDNA prepared in assay buffer) and incubated at 25° C. for 35 min. A forked dsDNA oligomer substrate was prepared by annealing equal amounts of oligo GPat-1 ([BHQ2]GCACTGGCCGTCGTTTTACGGTCGTGACT) and oligo GPat-2 (TTTTTTACTTAACGACGGCCAGTGC[TAM]) and was used as dsDNA substrate for WRN ATPase assay. A typical ATPase reaction consists of 25 nM forked dsDNA, 100 μM ATP, and 1 nM WRN prepared in assay buffer. The WRN ATPase reaction was stopped by the addition of 5 μL of ADP-Glo Reagent from ADP-Glo™ Kinase Assay kit and incubated for 40 min to deplete the unreacted ATP. Afterwards, 5 μL of Kinase Detection Reagent from the ADP-Glo™ Kinase Assay kit was added and incubated for 1 hr for the development of luminescence signal.

Luminesce signals were measured on BMG PHERAstar FSX using luminescence protocol. Data analysis was carried out using Genedata software following % of control normalization. Fitting parameters include dose response IC₅₀, Hill slope (n), maximum inhibition (S_{∞}), and minimum inhibition (S₀). The reported IC₅₀ values for compounds in this invention are the geometric means of at least 2 independent replicates.

Results of the WRN ATPase activity assay for compounds of the Examples are shown in Table 4.

TABLE 4
WRN ATPase activity of the compounds of the Examples.

| Example | |
|---|---|
| Compound | WRN ATPase Activity |
| 1 | + |
| 2 | + |
| 3 | +++ |
| 4 | +++ |
| 5 | + |
| 6 | + |
| 7 | ++ |
| 8 | + |
| 9 | +++ |
| 10 | + |
| 11 | +++ |
| 12 | + |
| 13 | + |
| 14 | + |
| 15 | + |
| 16 | + |
| 17 | + |
| 18 | +++ |
| 19 | ++ |
| 20 | ++ |
| 21 | ++ |
| 22 | + |
| 23 | ++ |
| 24 | ++ |
| 25 | + |

Note:
ATPase activity is represented by the following: + is <100 nM; ++ is 100 nM-<500 nM; +++ is 500 nM to <1000 nM; and ++++ is >1000 nM.

### Example B. WRN Helicase Activity Assay

Compounds of the examples were tested for WRN helicase activity. DNA encoding the target protein, 6×HIS-TEV-WRN-517-945, was produced, cloned and transfected as detailed in Example D. To determine the inhibitory properties of the compounds, a 384 wells plate-based Helicase assay was set up to measure the extent of DNA unwinding (Helicase) activity of WRN. The WRN Helicase assay was designed to measure increase in fluorescence signal using a fluorescence based, donor-quencher paired forked dsDNA oligomer substrate (custom synthesis from Sigma-Aldrich) prepared by annealing equal amounts of oligo GPat-1 ([BHQ2]GCACTGGCCGTCGTTTTACGGTCGTGACT) and oligo GPat-2 (TTTTTTACTTAACGACGGCCAGTGC[TAM]). Continuous measurement of fluorescent signal was used to optimize the buffer conditions, concentration of enzyme (WRN), concentration of substrates (ATP & dsDNA), and the linearity of the Helicase reaction. All solutions were prepared in the following assay buffer: 50 mM Tris-HCl, pH-7.5, 2 mM MgCl₂, 50 mM NaCl, 1 mM EGTA, 0.01% P-20, 0.05% BSA, and 2 mM DTT prepared with nuclease free water.

A serial dilution (1:3 scheme) of the compounds was prepared in neat DMSO and 100 nL of the compounds was dispensed in the Greiner black low volume (Greiner #784076) 384 wells assay plate. An 1-hr pre-incubation of the compounds with enzyme was included. A His-tagged construct of WRN (AA S2-S1235) was produced and used for the ATPase assay. Maximum inhibition control wells were set up with the addition of only assay buffer, and no inhibition control wells were set up with neat DMSO without test compounds. The reaction was initiated by addition of 5 μL substrate mix (200 μM ultrapure ATP, and 50 nM dsDNA prepared in assay buffer) and incubated at 25° C. for 35 min. A typical Helicase reaction consists of 25 nM forked dsDNA, 100 μM ATP, and 1 nM WRN prepared in assay buffer. The WRN Helicase reaction was quenched by the addition of 5 μL ‘STOP’ solution containing 100 mM EDTA (final EDTA concentration 33.33 mM) prepared in assay buffer. The plates were read on BMG PHERAstar FSX using 540/590 module to measure an increase of fluorescence. Data analysis was carried out using the Genedata software following % of control normalization. Fitting parameters include dose response IC₅₀, Hill slope (n), maximum inhibition (S_{∞}), and minimum inhibition (S₀). The reported IC₅₀ values for compounds in this invention are the geometric means of at least 2 independent replicates.

Results of the WRN Helicase activity assay for compounds of the Examples are presented in Table 5.

TABLE 5
WRN Helicase activity of the Examples.

| Example | |
|---|---|
| Compound | WRN Helicase Activity |
| 1 | + |
| 2 | + |
| 3 | ++++ |
| 4 | +++ |
| 5 | + |
| 6 | ++ |
| 7 | +++ |
| 8 | + |
| 9 | +++ |
| 10 | + |
| 11 | +++ |
| 12 | + |
| 13 | + |
| 14 | + |
| 15 | + |
| 16 | + |
| 17 | + |
| 18 | +++ |
| 19 | ++ |
| 20 | ++ |
| 21 | ++ |
| 22 | + |
| 23 | +++ |
| 24 | ++ |
| 25 | + |

Note:
Helicase activity is represented by the following: + is <100 nM; ++ is 100 nM-<500 nM; +++ is 500 nM to <1000 nM; ++++ is 1000 nM to 2000 nM; and +++++ is >2000 nM.

### Example C. WRN Inhibitor Cellular Activity Assays

Human colon carcinoma cell lines are obtained from ATCC, which included MSI-H cell lines RKO (CRL-2577) and SW-48 (CCL-231), and MSS cell lines SW-620 (CRL-227) and SW-837 (CRL-235). All cells are maintained in RPMI-1640 supplemented with 10% FBS (Gibco cat #11875119, 16140071) and will be kept at 37° C. in a humidified 5% CO₂ incubator. WRN inducible knockouts are generated using CRISPR-Cas9 editing and stable cell lines will be kept in media supplemented with antibiotic selection of 200 micrograms per milliliter hygromycin B (Gibco cat #10687010) and 1 microgram per milliliter puromycin (Gibco cat #A1113803).

### Cellular Assay—WRN Inhibitor Anti-Proliferation Assay

All cells are trypsinized, counted, and resuspended in RPMI-1640 with 10% FBS. 100 microliter cells and growth media are seeded into black 96 well plates (Greiner cat #655090). Seeding densities are 2,500 cells for RKO and SW-620, and 12,500 cells for SW-48 and SW-837. Following incubation to allow for cell adherence to the plate, compounds are added for dose response measurement, starting at the highest concentration, followed with a 3:1 serial dilution scheme. The last dose contains only DMSO and serves as no treatment control. Plates are incubated for three to seven days at 37° C. in a humidified 5% CO₂ incubator. Following incubation, 100 microliter Cell Titer Glo reagent (Promega cat #G7573) is added and plates are read on the Pherastar FSX (BMG Labtech). Data analysis are carried out using the Genedata software, following % of growth inhibition normalized to control (DMSO treated cells). Fitting parameters can include dose response IC₅₀, Hill slope (n), maximum response (S_{∞}), and minimum response (S₀).

Cellular Assay—WRN Inhibitor pKAP1 Induction Assay

Cells are trypsinized, counted, and resuspended in RPMI-1640 with 10% FBS. Cells re resuspended at 1×10⁶ cells per milliliter and ten microliters are added to regular volume Culturplate-384 well (Perkin Elmer cat #6007680) via the Multiflo (Biotek). Plates are wrapped to prevent evaporation and are incubated at 37° C. in a humidified 5% CO₂ incubator. The next day, compounds are added via the CyBio Felix (Analytik Jena) for dose response measurement, starting at the highest concentration, followed with a 3:1 serial dilution scheme. The last dose contains only DMSO and serves as no treatment control. Plates are wrapped to prevent evaporation and are incubated at 37° C. in a humidified 5% CO₂ incubator. The next day, cells are washed with the Blue washer (Blue Cat Bio) and AlphaLISA lysis buffer is added. Cells are allowed to lyse at room temperature for 30 min followed by the addition of pKAP1 AlphaLISA reagents (Perkin Elmer/Revvity cat #ALSU-PKAP1-A1OK). Plates are incubated in the dark until read on the Pherastar FSX (BMG Labtech).

Data analysis is carried out using the Genedata software. Activity data are normalized to percent of active control (WRN tool inhibitor compound). Fitting parameters can include dose response (EC₅₀), Hill slope (n), maximum response (S_{∞}), and minimum response (S₀).

### Example D—Protein Expression and Purification of WRN Protein for Biochemical Assays

DNA encoding a target WRN protein was synthetically produced with codon optimization for insect cell expression and subcloned into pFastBac1 at the indicated restriction sites by standard methods. Transfer vector DNA was isolated and verified by sequence analysis. E. coli strain DH10Bac was transformed with the transfer vector DNA to produce a recombinant bacmid. Bacmid DNA was isolated and confirmed to be recombinant by using standard PCR methods using M13 forward and reverse primers. Sf9 insect cells were transfected with recombinant bacmid DNA using Cellfectin II Reagent (Life Technologies, Cat. #58760) using standard methods. Baculovirus (P1) produced through transfection was amplified to obtain a high titer stock. Sf9 insect cells were seeded at an initial density of 2.3×10⁶ cells/mL and were then infected with high titer baculovirus stocks at an MOI=0.5 to 1.0. Cells were harvested 68 hrs post infection via centrifugation for 10 min at 500×g at 4° C.

Cell paste from 20 L of cell culture (˜171.5 g) was lysed via a freeze-thaw cycle and resuspended in 1600 mL of lysis buffer consisting of 20 mM HEPES, 500 mM NaCl, 10% glycerol, 20 mM imidazole, pH 7.5 (23° C.), 2 mM MgCl₂, 0.1% 3-OG, 0.1% DDM, 20 U/mL TurboNuclease and Complete™ EDTA-free-PI tablets (Roche, 2 tablets per 100 mL of buffer). Protein was extracted by stirring at 4° C. for 1.5 hrs. Lysate was clarified via centrifugation at 20,000×g for 45 min at 4° C.

The soluble lysate fraction was batch-bound to 50 mL of Ni-NTA His-Bind (EMD Millipore) resin at 4° C. for 1 hr while nutating. The resin was collected in a 5 cm diameter Econo-column and washed with 20 column volumes (CV) of Buffer A followed by 20 CV of Buffer B. The protein of interest was eluted in Buffer C. Fractions (25 mL) were collected and analyzed by A280 prior to pooling of the fractions. Select fractions were analyzed using SDS-PAGE. Buffer compositions were as follows: Buffer A: 20 mM HEPES, 500 mM NaCl, 10% glycerol, 20 mM imidazole, pH 7.5, Buffer B: Buffer A+20 mM imidazole, pH 7.5, Buffer C: Buffer A+280 mM imidazole, pH 7.5.

The pooled fractions were concentrated to 75 mL using a Vivacell 100 centrifugal device (30K molecular weight cutoff, PES membrane) and the resulting sample was loaded onto a Superdex 200 preparative Size Exclusion Chromatography column (5.0 cm×90 cm)(Cytiva). Fractions (13 mL) were collected over 1.1 CV and analyzed using SDS-PAGE prior to pooling. The pool fractions were concentrated using a Vivacell 100 centrifugal device (30K molecular weight cutoff, PES membrane) and flash frozen in liquid nitrogen and stored at −80° C.

### Example E—Protein Expression and Purification of WRN Protein for Crystal Structure Determination

DNA encoding the target protein, 6×HIS-TEV-WRN-517-945, was produced, cloned and transfected as detailed in Example D. 6 L batches of insect cell culture were produced and processed as detailed in Example D. The cell pellets were thawed and resuspended in IMAC buffer A (50 mM HEPES pH 7.5, 500 mM NaCl, 10% glycerol, 10 mM imidazole, 0.5 mM TCEP) supplemented with DNase, 1 mM PMSF and Complete protease inhibitor tablets (Roche). The resuspended cells were sonicated on ice for 10 min using 5 second on/off pulses at 30% amplitude. Cells were clarified using ultracentrifugation at 120,000×g for 60 min at 4° C.

The cleared lysate was loaded on to 3×5 ml HisTrap Excel columns (Cytiva) pre-equilibrated with IMAC buffer A and the column was washed with an increasing gradient of 2%, 4% and 6% of IMAC buffer B (IMAC buffer A+500 mM imidazole). Elution of the bound protein was performed using a linear gradient of IMAC buffer B ranging from 6% to 100% imidazole (30-500 mM imidazole). Fractions containing the protein of interest were combined and supplemented with TEV protease at a ratio of 1:20 TEV: protein (w/v), and dialyzed against 1 L of IMAC buffer A. After overnight cleavage (16 hrs), the sample was centrifuged to remove aggregates and loaded on to a HisTrap Excel column to capture the TEV protease, the His-tagged GST fragment, and the uncleaved material.

The flow-through and wash fractions from the IMAC purification were pooled and concentrated using a centrifugal concentrator with a 30 kDa molecular weight cutoff (Millipore). The concentrated sample was injected on to a HiLoad 16/600 Superdex 200 preparative Size Exclusion Chromatography column (Cytiva) equilibrated in SEC buffer (25 mM HEPES pH 7.5, 300 mM NaCl, 10% glycerol, 1 mM TCEP). The fractions were collected and analyzed using SDS-PAGE. Fractions corresponding to the WRN protein were combined and concentrated using a centrifugal concentrator with a 30 kDa molecular weight cut-off to 12-16 mg/mL (Millipore). The protein was aliquoted, flash frozen in liquid nitrogen, and stored at −80° C.

Co-Crystallization with WRN Inhibitor Compound

Crystallization of the purified WRN protein with a WRN inhibitor compound was performed using sitting drop vapor diffusion techniques. Crystals were obtained at 4° C. from the Wizard Screen (Molecular Dimensions) condition consisting of 0.1 M MES pH 6.0, 0.2M calcium acetate and 20% (w/v) PEG 8000. The condition was optimized by varying pH, calcium acetate and PEG concentrations. Furthermore, an additive screen was set up and the additives ammonium sulfate and sodium malonate were found to produce larger and more robust crystals for data collection. For co-crystal determination, WRN protein was co-crystallized in the presence of 2 mM of the WRN inhibitor compound. The crystals were subsequently cryo-protected in precipitant solution supplemented with 2 mM of the WRN inhibitor compound and 25% glycerol before flash-cooling in liquid nitrogen.

Data was collected at beamline 103, Diamond Light Source, Didcot, UK, at 100 K and λ=0.9763 Å. 3600 images were collected with an oscillation range of 0.1° per image. The beamline was equipped with a Dectris Eiger2 XE 16M detector. The data were processed using the autoPROC pipeline (1), which includes the software XDS (2), Aimless (3), and Pointless (4).

### Structure Refinement of Co-Crystal Structure

The WRN protein structure was determined by molecular replacement using Phaser (5) using a truncated model of the published WRN (PDB ID: 6YHR). To enable successful molecular replacement, each domain of the WRN protein was placed independently. The structure was refined using Buster (6) and model building was carried out in Coot (7). One WRN monomer was located in the asymmetric unit and initial difference density maps revealed that the WRN inhibitor compound was bound to the protein. Ligand coordinates and restraint files for use in Buster were generated using Grade2 from Global Phasing Ltd (8).

The structure of WRN in complex with the WRN inhibitor compound was determined to 3.1 Å resolution and includes amino acid residues 526-925 and 929-945 in chain A. The missing residues were all located in loop regions and were not visible in the electron density. Furthermore, one zinc ion and three sulphate ions were modelled into the electron density. The compound was overall well-defined in the electron density.

### Analysis of Co-Crystal Structure

The co-crystal structure of the WRN helicase core bound to the WRN inhibitor compound was determined to 3.1 A resolution. Analysis of the structure revealed an unexpected conformation, with a 150 degree rotation of the ATPase 2A domain relative to the ATPase 1A domain of the helicase core around the flexible linker. Previously published WRN helicase structures, including RCSB PDB ID: 6YHR, did not display this inter-domain rotation.

The 150 degree rotation of domain 2A of the helicase core results in rearrangement of residues necessary for ATP hydrolysis; specifically, the conserved Walker A motif (which is traditionally observed in the SF2 family of helicase proteins) is repositioned to form an unexpected pocket which was not included in any structures of the WRN helicase deposited in the Protein Data Bank. The conformational rearrangement also resulted in a new binding site in which Cys727 is in close proximity (3.3 angstroms) to the WRN inhibitor compound. In contrast, a corresponding linear sequence alignments of Walker A and other canonical motifs suggests Cys580 (within the Walker A motif) to be involved with the WRN inhibitor compound, and not the novel Cys727 binding site found in this co-crystal analysis. Additional novel interacting residues at Arg857 (Motif VI, the arginine finger, ATP binding site, (2)) and Phe730 (linker region between domains 1A and 2A) were also identified.

### Example F. Mutant Panel Shows Decoupling of ATPase and Helicase Activity Through Cys 727

Point mutations were made in the His-tagged WRN construct used for biochemical characterization (aa S2-S1235). Cysteine 580 (Walker A motif), 727 (linker), 864 (Domain 2 Å) and 1041 (RQC domain) were each mutated to both Serine and Alanine using site directed mutagenesis, for a total of eight mutants. Purified proteins were assayed in both helicase and ATPase assays. Cys580 mutation to either Ser or Ala abolished all activity, consistent with Walker A motif cysteine function. Cys864 (D2) mutation to alanine and Cys1041 (RQC domain) had no significant impact on helicase or ATPase activity. Cys727 abolished helicase activity (Table 6), whether it was mutated to Ala or Ser. ATPase activity for Cys727 mutants was reduced but not abolished (Table 7). Table 6 is the panel of the nine variant WRN helicase proteins that were screen using a flourscence-based DNA unwinding assay with an increase in flourscence intensity indicating DNA unwinding. Table 7 is the panel of the nine variant WRN helicase proteins that were screened using ADP-Glo assay to measure DNA stimulated ATPase activity, with an increase in luminescence indicating ATP hydrolysis.

TABLE 6
Helicase activity of WRN mutants.

| | Observed Helicase Activity | |
|---|---|---|
| WRN Constructs | (AU/min) | Ratio to WT |
| 1. C580A | 1.04 | 0.02 |
| 2. C580S | 0 | 0.00 |
| 3. C727A | 4.619 | 0.07 |
| 4. C727A | 1.452 | 0.02 |
| 5. C864A | 60.75 | 0.94 |
| 6. C864S | 28.49 | 0.44 |
| 7. C1041A | 60.14 | 0.93 |
| 8. C1041S | 40.28 | 0.62 |
| 9. WR WRN 12 | 64.77 | 1.00 |

TABLE 7
ATPase activity of WRN mutants.

| | Observed Helicase Activity | |
|---|---|---|
| WRN Constructs | (AU/min) | Ratio to WT |
| 1. C580A | 2.548 | 0.03 |
| 2. C580S | 0.8014 | 0.01 |
| 3. C727A | 22.65 | 0.29 |
| 4. C727A | 25.42 | 0.33 |
| 5. C864A | 70.52 | 0.90 |
| 6. C864S | 42.02 | 0.54 |
| 7. C1041A | 66.66 | 0.85 |
| 8. C1041S | 45.74 | 0.59 |
| 9. WR WRN 12 | 78.17 | 1.00 |

### Example G. The Compound of Example 1 Shows Time Dependent Inhibition and Reduction in Potency when Cys 727 is Mutated

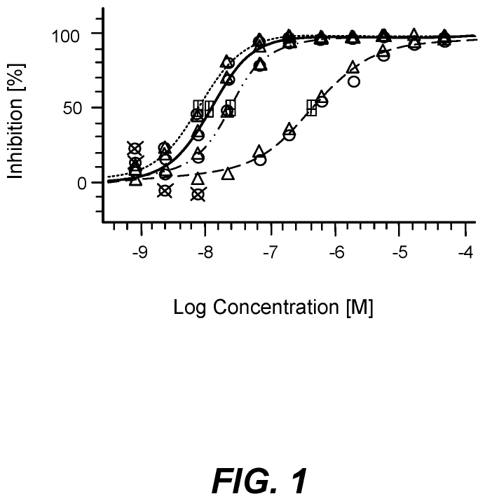

An ATPase assay on WRN helicase was performed using ADP-Glo and IC₅₀ values were determined by varying the compound of Example 1 preincubation times (FIG. 1). The IC₅₀ was determined to be 449 nM without preincubation. Time dependent inhibition was demonstrated by decreasing IC₅₀ values with increasing preincubation time. IC₅₀ values were determined to be: 24 nM (1 hour), 12 nM (2 hrs), 8 nM (4 hrs, FIG. 1). In FIG. 1, 0 hrs is represented by bottom dotted line; 1 hour is represented by the broken line with two bullets; 2 hrs is represented by solid line; and 4 hrs is represented by top dashed line.

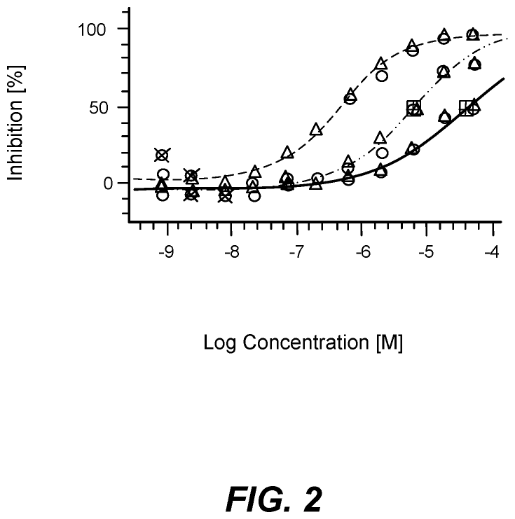

Furthermore, mutation of Cys727 (Cys727Ala and Cys727Ser) were shown to reduce potency (FIG. 2). From FIG. 2, the top line represented WT. Cys727Ala results in an IC₅₀ of 5818 nM (middle line in FIG. 2) which represent a 13-fold shift relative to WT, and Cys727Ser results in an IC₅₀ of 38,484 nM (bottom line in FIG. 2) which represents an 86-fold shift relative to WT. The mutant experiments were performed without preincubation.

### Example H. Intact Mass Spectrometry Verifies Covalent Modification

Intact mass spectrometry suggests the formation of a 1:1 adduct of WRN helicase and Example 1. The intact WRN helicase has an observed mass of 67,573.1 Da (theoretical mass of 67,409.4 Da). The WRN helicase incubated with the compound of Example 1 has an observed mass of 68,164.5 (theoretical mass of 1:1 adduct 68,000 Da). The shift in the observed mass between intact WRN and WRN incubated with the compound of Example 1 is equivalent to the theorectical mass of the compound of Example 1 (592.2 Da). Peptide mapping analysis further confirmed that Cys727 is the only residue to be modified by the compound of Example 1. The mass spectrometry results, combined with the ATPase assay results established that covalently targeting Cys727 is and effective and tractable way to inhibit both the ATPase and helicase activities of WRN.

Various modifications of the contents of the present disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula (I): 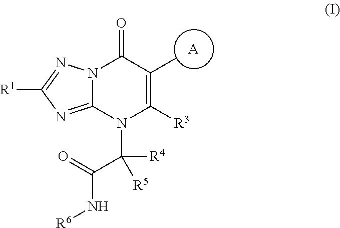
or a pharmaceutically acceptable salt thereof, wherein:
each [symbol] is independently a single or double bond, provided that proper valency is maintained;
Ring moiety A is selected from Formulas (A-1), (A-2), (A-3), (A-4), (A-5), and (A-6): 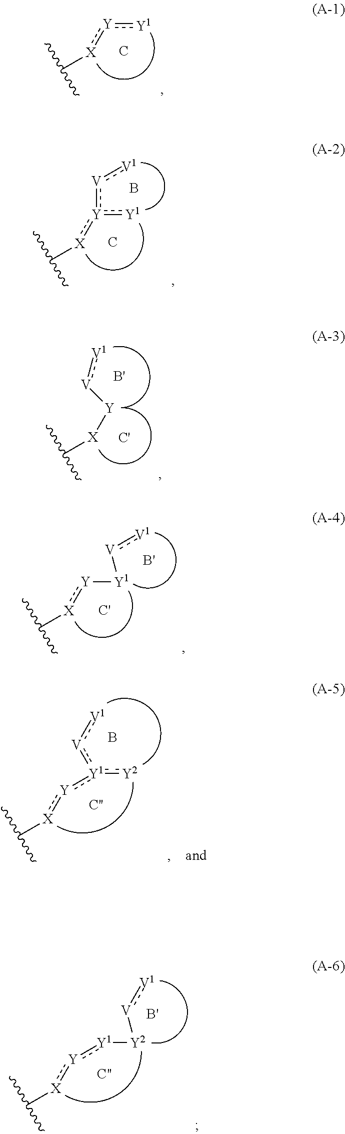
wherein Ring C is monocyclic in Formula (A-1), Ring B is fused to Y and Y¹ of Ring C in Formula (A-2), Ring B′ is spiro bound to Y in Ring C′ in Formula (A-3), Ring B′ is spiro bound to Y¹ of Ring C′ in Formula (A-4), Ring B is fused to Y¹ and Y² of Ring C″ in Formula (A-5), and Ring B′ is spiro bound to Y² in Ring C″ in Formula (A-6);
Ring B is selected from phenyl, C₃₋₇ cycloalkyl, 5-6-membered heteroaryl, or 4-7-membered heterocycloalkyl, which is optionally substituted by 1, 2, 3, or 4 independently selected R² substituents;
Ring C is selected from phenyl, C₃₋₇ cycloalkyl, 5-6-membered heteroaryl, or 4-7 membered heterocycloalkyl, which is optionally substituted by 1, 2, 3, or 4 independently selected R² substituents;
Ring B′ is 4-7-membered heterocycloalkyl or C₃₋₇ cycloalkyl, which is optionally substituted by 1, 2, 3, or 4 independently selected R² substituents;
Ring C′ is selected from 4-7 membered heterocycloalkyl or C₃₋₇ cycloalkyl, which is optionally substituted by 1, 2, 3, or 4 independently selected R² substituents;
Ring C″ is selected from 4-7 membered heterocycloalkyl or C₄₋₇ cycloalkyl, which is optionally substituted by 1, 2, 3, or 4 independently selected R² substituents;
X is C, CH, or N;
when Ring A is Formula (A-1), then (i) Y is CR^{W}, CHR^{W}, or NR^{W}; and Y¹ is CH, CH₂, N, NH, S or O; or (ii) Y is CH, CH₂, N, NH, S or O; and Y¹ is CR^{W}, CHR^{W}, or NR^{W}; provided that there are no O—O, S—S, or S—O bonds;
when Ring A is Formula (A-2), then (i) Y is C, CH, or N; Y¹ is C, CH, or N; V is CR^{W}, CHR^{W}, or NR^{W}; and V¹ is CH, CH₂, N, NH, S or O; or (ii) Y is C, CH, or N; Y¹ is C, CH, or N; V is CH, CH₂, N, NH, S or O; and V¹ is CR^{W}, CHR^{W}, or NR^{W}; provided that there are no O—O, S—S, or S—O bonds;
when Ring A is Formula (A-3), then (i) Y is C; V is CR^{W}, CHR^{W}, or NR^{W}; and V¹ is CH, CH₂, N, NH, S or O; or (ii) Y is C; V is CH, CH₂, N, NH, S or O; and V¹ is CR^{W}, CHR^{W}, or NR^{W}; provided that there are no O—O, S—S, or S—O bonds;
when Ring A is Formula (A-4), then (i) Y is CH, CH₂, N, NH, S or O; Y¹ is C; V is CR^{W}, CHR^{W}, or NR^{W}; and V¹ is CH, CH₂, N, NH, S or O; or (ii) Y is CH, CH₂, N, NH, S or O; Y¹ is C; V is CH, CH₂, N, NH, S or O; and V¹ is CR^{W}, CHR^{W}, or NR^{W}; or (iii) Y is CR^{W}, CHR^{W}, or NR^{W}; Y¹ is C; V is CH, CH₂, N, NH, S or O; and V¹ is CH, CH₂, N, NH, S or O; provided that there are no O—O, S—S, or S—O bonds;
when Ring A is Formula (A-5), then (i) Y is CR^{W}, CHR^{W}, or NR^{W}; Y¹ is C, CH, or N; Y² is C, CH, or N; V is CH, CH₂, N, NH, S or O; and V¹ is CH, CH₂, N, NH, S or O; or (ii) Y is CH, CH₂, N, NH, S or O; Y¹ is C, CH, or N; Y² is C, CH, or N; V is CR^{W}, CHR^{W}, or NR^{W}; and V¹ is CH, CH₂, N, NH, S, O, or a bond; or (iii) Y is CH, CH₂, N, NH, S or O; Y¹ is C, CH, or N; Y² is C, CH, or N; V is CH, CH₂, N, NH, S or O; and V¹ is CR^{W}, CHR^{W}, or NR^{W}; provided that there are no O—O, S—S, or S—O bonds;
when Ring A is Formula (A-6), then (i) Y is CR^{W}, CHR^{W}, or NR^{W}; Y¹ is CH, CH₂, N, NH, S or O; Y² is C; V is CH, CH₂, N, NH, S or O; and V¹ is CH, CH₂, N, NH, S or O; or (ii) Y is CH, CH₂, N, NH, S or O; Y¹ is CR^{W}, CHR^{W}, or NR^{W}; Y² is C; V is CH, CH₂, N, NH, S or O; and V¹ is CH, CH₂, N, NH, S or O; or (iii) Y is CH, CH₂, N, NH, S or O; Y¹ is CH, CH₂, N, NH, S or O; Y² is C; V is CR^{W}, CHR^{W}, or NR^{W}; and V¹ is CH, CH₂, N, NH, S or O; or (iv) Y is CH, CH₂, N, NH, S or O; Y¹ is CH, CH₂, N, NH, S or O; Y² is C; V is CH, CH₂, N, NH, S or O; and V¹ is CR^{W}, CHR^{W}, or NR^{W}; provided that there are no O—O, S—S, or S—O bonds;
R¹ is independently selected from H, D, halo, CN, NO₂, C₁₋₆ alkyl, C₂₋₆ alkenyl, C₂₋₆ alkynyl, C₁₋₆ haloalkyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄alkyl, OR^{a1}, SR^{a1}, NHOR^{al}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{c1}R^{g1}, OP(O)(OR^{h1})(ORⁱ¹), P(O)(OR^{h1})(ORⁱ¹), and BRO^{j1}R^{k1}, wherein said C₁₋₆ alkyl, C₂₋₆ alkenyl, C₂₋₆ alkynyl, C₁₋₆ haloalkyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{1A} substituents
or, any R^{c1} and R^{d1} attached to the same N atom, together with the N atom to which they are attached, form a 4-10 membered heterocycloalkyl group, wherein the 4-membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-membered heteroaryl-C₁₋₄alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11} SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11} S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j1}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a12} SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{c12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{c12})R^{b12}, OS(O)₂R^{b12}, S(O)(═NR^{c12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is independently selected from H, D, halo, CN, NO₂, C₁₋₆ alkyl, C₂₋₆ alkenyl, C₂₋₆ alkynyl, C₁₋₆ haloalkyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{c2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{c2})R^{b2}, OS(O)₂R^{b2}, S(O)(═NR^{c2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{2A} substituents
or, any R^{c2} and R^{d2} attached to the same N atom, together with the N atom to which they are attached, form a 4-10 membered heterocycloalkyl group, wherein the 4-membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-membered heteroaryl-C₁₋₄alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21} SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21} S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₃R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₃NR^{c3}R^{d3}, OS(O)(═NR^{e3})R^{b3}, OS(O)₂R^{b3}, S(O)(═NR^{e3})R^{b3} SF₅, P(O)R^{f3}R^{g3}, OP(O)(OR^{h3})(ORⁱ³), P(O)(OR^{h3})(ORⁱ³), and BR^{j3}R^{k3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f3} and R^{g3} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h3} and Rⁱ³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁴ and R⁵ are each independently selected from H, C₁₋₆ alkyl, C₁₋₆ haloalkyl, cyano-C₁₋₃ alkyl, HO—C₁₋₃ alkyl, and C₁₋₃ alkoxy-C₁₋₃ alkyl; or
any R⁴ and R⁵ are, together with the carbon atom to which they are attached, form a 3-7-membered cycloalkyl ring;
R⁶ is independently selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, C(O)R^{b6}, C(O)NR^{c6}R^{d6}, C(O)NR^{c6}(OR^{a6}), C(O)OR^{a6}, C(═NR^{e6})R^{b6}, C(═NR^{e6})NR^{c6}R^{d6}, S(O)R^{b6}, S(O)NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄ alkyl are each optionally substituted with 1, 2, 3, or 4 independently selected R^{6A} substituents;
each R^{a6}, R^{c16}, and R^{d6} is independently selected from H, C₁₋₆ alkyl, C₁₋₆ haloalkyl, C₂₋₆ alkenyl, C₂₋₆ alkynyl, C₃₋₁₀cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{6A} substituents
or, any R^{c6} and R^{d6} attached to the same N atom, together with the N atom to which they are attached, form a 4-10 membered heterocycloalkyl group, wherein the 4-membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-membered heteroaryl-C₁₋₄ alkyl;
each R^{6A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61} SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{e61})R^{b61}, OS(O)₂R^{b61}, S(O)(═NR^{e61})R^{b61}, SF₅, P(O)R^{f61}R^{g61}, OP(O)(OR^{h61})(ORⁱ⁶¹), P(O)(OR^{h61})(ORⁱ⁶¹), and BR^{j61}R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62} SR^{a62}, NHOR^{a62}, C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{a62}, S(O)R^{b62}, S(O)NR^{c62}R^{a62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is: 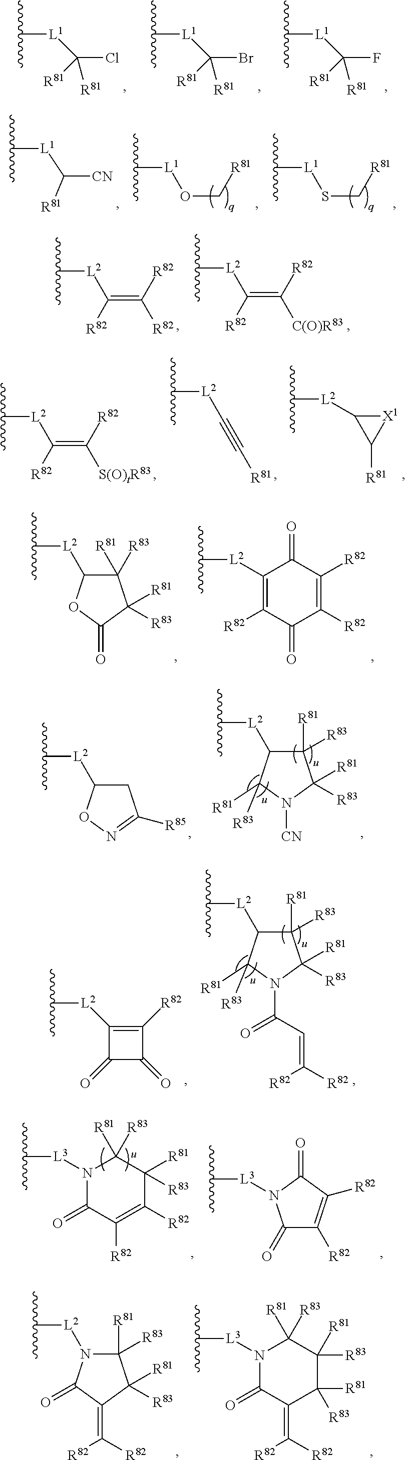 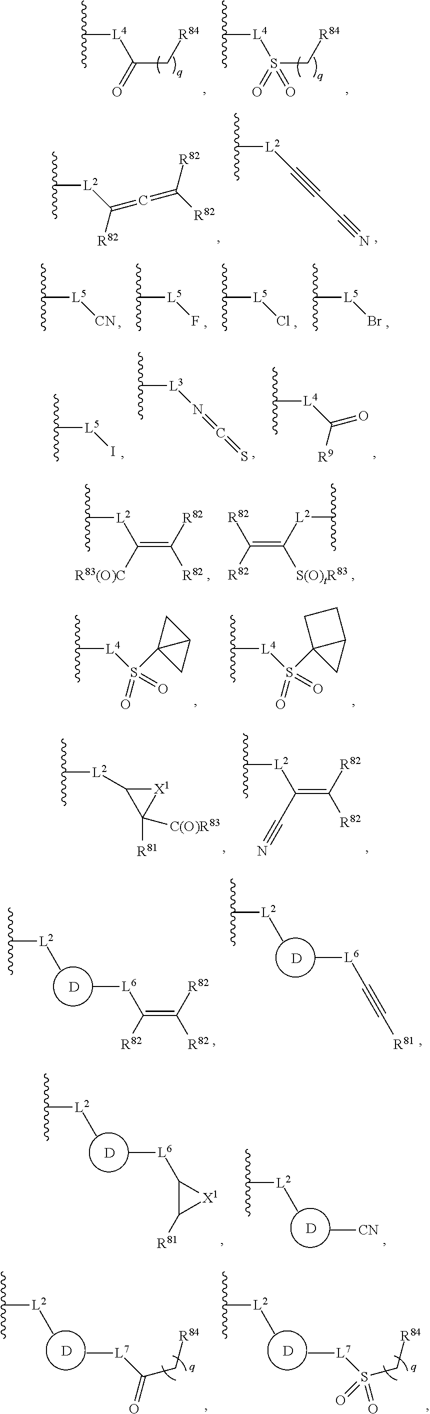
or L¹-Ar;
L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Ring moiety A through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Ring moiety A through the L linking group;
L³ is -L-, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L³ is attached to Ring moiety A through the L linking group;
L⁴ is -L-, -L-O—, L-S—, or -L-NR⁹—, wherein L⁴ is attached to Ring moiety A through the L linking group;
L⁵ is -L-O-L^{x}-, -L-NR⁹-L^{x}-, -L-S-L^{x}-, -L-C(O)-L^{x}-, -L-NR⁹C(O)-L^{x}-, -L-OC(O)-L^{x}-, -L-S(O)-L^{x}-, -L-S(O)₂-L^{x}-, -L-NR⁹S(O)-L^{x}-, -L-OS(O)-L^{x}-, -L-NR⁹S(O)NR⁹-L^{x}-, -L-NR⁹S(O)O-L^{x}-, -L-OS(O)NR⁹-L^{x}-, -L-NR⁹S(O)₂-L^{x}-, -L-OS(O)₂-L^{x}-, -L-NR⁹S(O)₂NR⁹-L^{x}-, -L-NR⁹S(O)₂O-L^{x}-, -L-S(O)(NR⁹)-L^{x}-, -L-S(O)₂(NR⁹)-L^{x}-, or -L-OS(O)₂NR⁹-L^{x}-, wherein L⁵ is attached to Ring moiety A through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—;
L⁷ is -L-, -L-O—, L-S—, or -L-NR⁹—;
each L is independently a bond or C₁₋₆ alkylene, wherein said C₁₋₆ alkylene is optionally substituted by 1, 2, 3 or 4 independently selected R^{G} substituents; or
each L is —O—C₁₋₆ alkyl or —N(RN)_C₁₋₆ alkyl, wherein L is attached to Ring moiety A via the oxygen of the —O—C₁₋₆ alkyl or the nitrogen atom of the —N(R^{N})_C₁₋₆ alkyl group;
each R^{N} is independently H or C₁₋₆ alkyl;
each L^{x} is independently a C₁₋₆ alkylene, wherein said C₁₋₆ alkylene is optionally substituted by 1, 2, 3 or 4 independently selected R^{G} substituents;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
X¹ is O or NR⁹;
each q is independently 0, 1, 2, or 3;
each t is independently 0, 1, 2, or 3;
each u is, independently, 0, 1, 2, or 3;
Ar is C₆₋₁₀ aryl or 5-10 membered heteroaryl, which are each optionally substituted with 1, 2, 3, or 4 independently selected R⁹ substituents;
each R⁸¹, and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8} SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{b8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄alkyl, and 5-6 membered heteroaryl-C₁₋₄alkyl, wherein said C₁₋₆ alkyl, C₂₋₆ alkenyl, C₂₋₆ alkynyl, C₁₋₆ haloalkyl C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄alkyl, 4-7 membered heterocycloalkyl-C₁₋₄alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substitutents;
each R⁸⁴ is independently H, D, halo, CN, OH, C₁₋₃ alkoxy, C₁₋₃ haloalkoxy, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₃ alkoxy, C₁₋₃ haloalkoxy, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are optionally substituted by 1, 2, 3, or 4 independently selected R^{G} substituents;
each R⁸⁵ is independently H, D, halo, CN, C(O)H, OH, C₁₋₃ alkoxy, C₁₋₃ haloalkoxy, amino, C₁₋₆ alkyl, C₁₋₆ haloalkyl, C₃₋₇ cycloalkyl, 4-7 membered heterocycloalkyl, or C₃₋₇ cycloalkyl-C₁₋₄alkyl, wherein said C₁₋₆ alkyl, C₁₋₆ haloalkyl, C₃₋₇ cycloalkyl, 4-7 membered heterocycloalkyl, and C₃₋₇ cycloalkyl-C₁₋₄ alkyl are each optionally substituted by 1, 2, 3, or 4 independently selected R^{G} substituents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, and 5-6 membered heteroaryl-C₁₋₄ alkyl, OR^{a9}, SR^{a9} NHOR^{a9}, C(O)R^{b9}, C(O)NR^{c9}R^{d9}, C(O)NR⁹(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R^{b9} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2-3. (canceled)

4. The compound of claim 1, or a pharmaceutically acceptable salt thereof, wherein:
Ring moiety A is selected from: 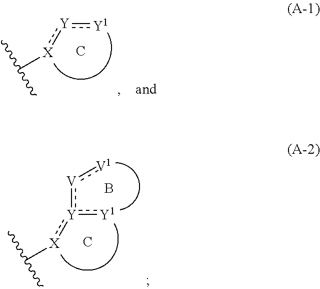
and
when Ring A is Formula (A-1), then Y is CR^{W}, CHR^{W}, or NR^{W}; and Y¹ is CH, CH₂, N, or NH; and
when Ring A is Formula (A-2), then Y is C, CH, or N; Y¹ is C, CH, or N; V is CR^{W}, CHR^{W}, or NR^{W}; and V¹ is CH, CH₂, N, or NH.

5-10. (canceled)

11. The compound of claim 1, or a pharmaceutically acceptable salt thereof, wherein X is C.

12. The compound of claim 1, or a pharmaceutically acceptable salt thereof, wherein Ring B is 5-6-membered heteroaryl or 5-6 membered heterocycloalkyl, each of which is optionally substituted by 1, 2, 3, or 4 independently selected R² substituents.

13-15. (canceled)

16. The compound of claim 1, or a pharmaceutically acceptable salt thereof, wherein Ring C is phenyl, 5-6-membered heteroaryl, or 5-6-membered heterocycloalkyl, each of which is each optionally substituted by 1, 2, 3, or 4 independently selected R² substituents.

17-24. (canceled)

25. The compound of claim 1, or a pharmaceutically acceptable salt thereof, wherein R¹ is independently selected from C₁₋₆ alkyl, C₂₋₆ alkenyl, C₂₋₆ alkynyl, C₃₋₇ cycloalkyl, phenyl, 4-7 membered heterocycloalkyl, and 5-6 membered heteroaryl, each of which are each optionally substituted with 1, 2, 3, or 4 independently selected R^{1A} substituents.

26. (canceled)

27. The compound of claim 1, or a pharmaceutically acceptable salt thereof, wherein:
each R^{a1}, R^{c1}, and R^{d1} is independently selected from H, C₁₋₆ alkyl, C₂₋₆ alkenyl, C₂₋₆ alkynyl, C₁₋₆ haloalkyl, and C₃₋₄cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{1A} substituents; and
each R^{b1} is independently selected from C₁₋₆ alkyl, C₂₋₆ alkenyl, C₂₋₆ alkynyl, C₁₋₆ haloalkyl, and C₃₋₄ cycloalkyl, which are each optionally substituted with 1, 2, 3, or 4 independently selected R^{1A} substituents.

28-29. (canceled)

30. The compound of claim 1, or a pharmaceutically acceptable salt thereof, wherein each R^{1A} is independently selected from D, OH, NO₂, CN, halo, C₁₋₃ alkyl, C₂₋₃ alkenyl, C₂₋₃ alkynyl, C₁₋₃ haloalkyl, cyano-C₁-3 alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

31. The compound of claim 1, or a pharmaceutically acceptable salt thereof, wherein R² is independently selected from H, D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄alkyl, 4-10 membered heterocycloalkyl-C₁-4 alkyl, 5-10 membered heteroaryl-C₁₋₄alkyl, OR^{a2}, SR^{a2}, NHOR^{a2}, C(O)R^{b2}C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄alkyl, and 5-10 membered heteroaryl-C₁₋₄alkyl are each optionally substituted with 1, 2, 3, or 4 independently selected R^{2A} substituents.

32-33. (canceled)

34. The compound of claim 1, or a pharmaceutically acceptable salt thereof, wherein:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄alkyl, and 5-6 membered heteroaryl-C₁₋₄alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄alkyl, and 5-6 membered heteroaryl-C₁₋₄alkyl are each optionally substituted with 1, 2, 3, or 4 independently selected R^{2A} substituents; and
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents.

35-36. (canceled)

37. The compound of claim 1, or a pharmaceutically acceptable salt thereof, wherein:
each R^{2A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄alkyl, phenyl-C₁₋₄alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents; and
each R^{2B} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

38-44. (canceled)

45. The compound of claim 1, or a pharmaceutically acceptable salt thereof, wherein R³ is independently selected from D, OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino.

46. The compound of claim 1, or a pharmaceutically acceptable salt thereof, wherein R³ is selected from C₁₋₆ alkyl, C₂₋₆ alkenyl, C₂₋₆ alkynyl, C₁₋₆ haloalkyl, cyano-C₁₋₆ alkyl, HO—C₁₋₆ alkyl, and C₁₋₆ alkoxy-C₁₋₆ alkyl.

47-48. (canceled)

49. The compound of claim 1, or a pharmaceutically acceptable salt thereof, wherein:
R⁴ and R⁵ are each independently selected from H, and C₁₋₆ alkyl; or
R⁴ and R⁵, together with the carbon atom to which they are attached, form a 3-7-membered cycloalkyl ring.

50-53. (canceled)

54. The compound of claim 1, or a pharmaceutically acceptable salt thereof, wherein R⁶ is independently selected from C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, each of which is optionally substituted by 1, 2, 3 or 4 independently selected R^{6A} substituents.

55-62. (canceled)

63. The compound of claim 1, or a pharmaceutically acceptable salt thereof, wherein each R^{6A} is independently selected from OH, CN, halo, C₁₋₆ alkyl, C₂₋₆ alkenyl, C₂₋₆ alkynyl, C₁₋₆ haloalkyl, cyano-C₁₋₆ alkyl, HO—C₁₋₆ alkyl, C₁₋₆ alkoxy-C₁₋₆ alkyl, C₃₋₆ cycloalkyl, C₁₋₆ alkoxy, C₁₋₃ haloalkoxy, amino, C₁₋₆ alkylamino, and di(C₁₋₆ alkyl)amino.

64. The compound of claim 1, or a pharmaceutically acceptable salt thereof, wherein each R^{6A} is independently selected from halo and C₁₋₆ haloalkyl.

65. (canceled)

66. The compound of claim 1, or a pharmaceutically acceptable salt thereof, wherein R^{W} is selected from:

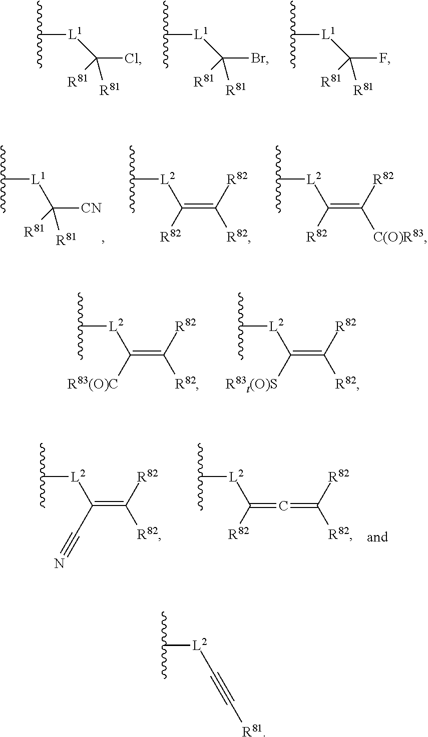

67. The compound of claim 1, or a pharmaceutically acceptable salt thereof, wherein R^{W} is

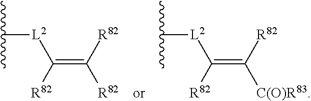

68. The compound of claim 1, or a pharmaceutically acceptable salt thereof, wherein L² is -L-C(O)—, -L-NR⁹C(O)— or -L-NR⁹S(O)₂— wherein L² is attached to Ring moiety A through an L linking group.

69. (canceled)

70. The compound of claim 1, or a pharmaceutically acceptable salt thereof, wherein each L is a bond.

71-74. (canceled)

75. The compound of claim 1, or a pharmaceutically acceptable salt thereof, wherein:
each R⁸¹ and R⁸³ is independently selected from H, D, halo, CN, C₁₋₆ alkyl, C₂₋₆ alkenyl, C₂₋₆ alkynyl, C₁₋₆ haloalkyl, C₃₋₇ cycloalkyl, C₁₋₆ alkoxy, C(O)R^{b8}, C(O)NR^{c8}R^{d8}, and C(O)OR^{a8};
each R⁸² is independently selected from H, D, halo, CN, C₁₋₆ alkyl, C₂₋₆ alkenyl, C₂₋₆ alkynyl, C₁₋₆ haloalkyl, C₃₋₇ cycloalkyl, C(O)R^{b8}, C(O)NR^{c8}R^{d8}, and C(O)OR^{a8}; and
each R^{a8}, R^{c8}, and R^{d8} is independently selected from H, C₁₋₆ alkyl, and C₃₋₇ cycloalkyl.

76. The compound of claim 1, or a pharmaceutically acceptable salt thereof, wherein each R⁸¹ and R⁸³ is independently selected from H, CN, C₁₋₆ alkyl, and C₁₋₆ alkoxy; and each R⁸² is independently selected from H, CN, and C₁₋₆ alkyl.

77-79. (canceled)

80. The compound of claim 1, or a pharmaceutically acceptable salt thereof, wherein R⁹ is H or methyl.

81. The compound of claim 1, selected from:
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
2-(2-(3,6-dihydro-2H-pyran-4-yl)-5-ethyl-7-oxo-6-(2-(vinylsulfonamido)phenyl)-[1,2,4]triazolo[1,5-a]pyrimidin-4(7H)-yl)-N-(4-(trifluoromethyl)phenyl)acetamide;
ethyl (E)-4-((2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)amino)-4-oxobut-2-enoate;
(E)-2-cyano-N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-4-methylpent-2-enamide;(Z)-2-cyano-N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-4-methylpent-2-enamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)-N-methylacrylamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-6-methylphenyl)acrylamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-5-methylphenyl)acrylamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-4-methylphenyl)acrylamide;
N-(5-chloro-2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(4-chloro-2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-5-(trifluoromethyl)phenyl)acrylamide;
N-(2-(2-(3,6-dihydro-2H-pyran-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)-5-fluorophenyl)acrylamide;
2-(6-(1-acryloyl-1,4,5,6-tetrahydropyridin-2-yl)-2-(3,6-dihydro-2H-pyran-4-yl)-5-ethyl-7-oxo-[1,2,4]triazolo[1, 5-a]pyrimidin-4(7H)-yl)-N-(4-(trifluoromethyl)phenyl)acetamide;
2-(6-(1-acryloylindolin-7-yl)-2-(3,6-dihydro-2H-pyran-4-yl)-5-ethyl-7-oxo-[1,2,4]triazolo[1,5-a]pyrimidin-4(7H)-yl)-N-(4-(trifluoromethyl)phenyl)acetamide;
N-(2-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)phenyl)acrylamide;
N-(2-(2-cyclopropyl-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(2-(5-ethyl-2-methyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(2-(2-cyclobutyl-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(2-(2-cyclohexyl-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide;
N-(2-(4-(2-((2-chloro-4-(trifluoromethyl)phenyl)amino)-2-oxoethyl)-5-ethyl-2-isopropyl-7-oxo-4,7-dihydro-[1,2,4]triazolo[1,5-a]pyrimidin-6-yl)phenyl)acrylamide;
N-(2-(5-ethyl-7-oxo-4-(2-oxo-2-((4-(trifluoromethyl)phenyl)amino)ethyl)-2-(thiophen-2-yl)-4,7-dihydro-[1,2,4]triazolo[1,5-a]pyrimidin-6-yl)phenyl)acrylamide;
N-(2-(5-ethyl-7-oxo-4-(2-oxo-2-((4-(trifluoromethyl)phenyl)amino)ethyl)-2-(tetrahydro-2H-pyran-4-yl)-4,7-dihydro-[1,2,4]triazolo[1,5-a]pyrimidin-6-yl)phenyl)acrylamide;
N-(2-(5-ethyl-7-oxo-4-(2-oxo-2-((4-(trifluoromethyl)phenyl)amino)ethyl)-2-(piperidin-4-yl)-4,7-dihydro-[1,2,4]triazolo[1,5-a]pyrimidin-6-yl)phenyl)acrylamide; and
N-(2-(2-(1-acryloylpiperidin-4-yl)-5-ethyl-7-oxo-4-(2-oxo-2-((4-(trifluoromethyl)phenyl)amino)ethyl)-4,7-dihydro-[1,2,4]triazolo[1,5-a]pyrimidin-6-yl)phenyl)acrylamide.

82. A pharmaceutical composition comprising the compound of claim 1, or a pharmaceutically acceptable salt thereof, and a pharmaceutically acceptable carrier.

83. A method of inhibiting WRN, comprising contacting the WRN with a compound of claim 1, or a pharmaceutically acceptable salt thereof.

84. A method of inhibiting WRN in a patient, comprising administering to the patient a compound of claim 1, or a pharmaceutically acceptable salt thereof, or a pharmaceutical composition comprising the compound of claim 1 or a pharmaceutically acceptable salt thereof, and a pharmaceutically acceptable carrier.

85. The method of claim 83, wherein the inhibition of WRN comprises the inhibition of: (i) WRN helicase activity; (ii) WRN ATPase activity; or (iii) both (i) and (ii).

86. A method of treating a disease or disorder associated with WRN activity in a patient, the method comprising administering to the patient in need thereof a therapeutically effective amount of the compound of claim 1, or pharmaceutically acceptable salt thereof, or a pharmaceutical composition comprising the compound of claim 1 or a pharmaceutically acceptable salt thereof, and a pharmaceutically acceptable carrier.

87. The method of claim 86, wherein the disease or disorder is cancer.

88. The method of claim 87, wherein the cancer is a microsatellite instability-high (MSI-H) cancer.

89. The method of claim 87, wherein the cancer is a mismatch repair deficient (dMMR) cancer.

90-94. (canceled)
